(19) United States
(12) Patent Application Publication
(10) Pub. No.: US 2026/0207732 A1
(43) Pub. Date: Jul. 23, 2026

(54) NOVEL INFLUENZA ANTIGENS

(71) Applicant: GLAXOSMITHKLINE BIOLOGICALS SA, Rixensart (BE)

(72) Inventors: Normand BLAIS, Rixensart (BE); Lionel SACCONNAY, Rixensart (BE); Ventzislav VASSILEV, Rixensart (BE)

(73) Assignee: GLAXOSMITHKLINE BIOLOGICALS SA, Rixensart (BE)

(21) Appl. No.: 19/140,700

(22) PCT Filed: Dec. 18, 2023
(86) PCT No.: PCT/EP2023/086328

§ 371 Date: Jun. 18, 2025

(30) Foreign Application Priority Data
Dec. 20, 2022 (GB) 2219228.0

(51) Int. Cl. A61K 39/145; A61K 39/00; A61P 37/04

(52) U.S. Cl. CPC A61K 39/145; A61P 37/04; A61K 2039/53; A61K 2039/645

(57) ABSTRACT

Immunogenic compositions comprising influenza A strain haemagglutinin antigens and polynucleotides encoding the antigens.

### TECHNICAL FIELD

The present invention relates to novel influenza virus antigens, nucleotide sequences encoding them, novel immunogenic or vaccine compositions, and uses of and methods for producing the antigens and compositions. In particular, the invention relates to immunogenic compositions comprising modified forms of influenza haemagglutinin (HA) from influenza A strain or nucleotide sequences encoding them, methods for their manufacture, and their use in the prevention of influenza virus A infections.

### BACKGROUND OF THE INVENTION

Influenza viruses have a significant impact on global public health, causing millions of cases of severe illness each year, thousands of deaths, and considerable economic losses. Influenza viruses belong to the family Orthomyxoviridae, a family that represents enveloped viruses, the genome of which comprises segmented, negative, single-strand RNA. Influenza viruses are divided into three main types that infect humans: influenza A virus, influenza B virus and influenza C virus. Influenza A and B are the types that are most clinically relevant to humans and are responsible for the flu season each year. Influenza type C infections generally cause mild illness and are not thought to cause human flu epidemics. Influenza strains are classified according to host species of origin, geographic site and year of isolation, serial number and for influenza A, by serological properties of subtypes of the two predominant surface glycoproteins HA and neuraminidase (NA). It is these surface proteins, particularly HA, that determine the antigenic specificity of the influenza subtypes or lineages.

Influenza A and influenza B diverged from each other around 2000 years ago and have structural similarities but a low sequence identity in the HA (Ni et al, Biochemistry, 2014, 53: 846-854).

Influenza B virus was first isolated in 1940, and since the 1980s two genetic lineages have been identified based on the antigenic properties of the HA: B/Victoria/2/87 (B/Vic) and B/Yamagata/16/88 (B/Yam).

Influenza B virus strains generally evolve more slowly in terms of genetic and antigenic properties than A strains. Influenza A viruses evolve and undergo antigenic variability continuously and have been responsible for past influenza pandemics.

Vaccination plays a critical role in controlling influenza epidemics and pandemics. Because of the antigenic variability, annual vaccinations are required to provide immunity against the influenza viruses that are in circulation. These are predicted based on viral surveillance data. Selection of the appropriate vaccine strains presents many challenges and frequently results in sub-optimal protection, since current influenza vaccines are primarily strain specific. Current seasonal influenza vaccines are trivalent (TIV), containing two A strain and one B strain virus, or quadrivalent (QIV), containing two A and two B strain viruses. QIVs contain both a B/Victoria and a B/Yamagata influenza B strain and TIVs contain one influenza B strain, from either the Victoria or Yamagata lineage.

The immune response to the current vaccines is largely directed against the highly variable HA. HA is a trimeric protein in which each monomer contains two polypeptide chains, HA1 and HA2, linked by a disulphide bond and anchored in the virus envelope by a C-terminus transmembrane domain.

Each monomer is initially expressed as inactive HA0 which is subsequently cleaved by host proteases into HA1 and HA2 subunits which are linked via a disulphide bond to form a metastable prefusion state HA. The triggering event for conversion of HA from prefusion to postfusion conformation has been linked to pH change (drop) upon viral uptake/endocytosis leading to membrane fusion and virus internalization. Although there are conserved features shared between influenza A and influenza B virus in the conformational transition between pre and postfusion conformation, there are substantial differences that influence the detailed mechanisms of this process (Ni et al, Biochemistry, 2014, 53: 846-854).

HA can be divided functionally into two domains, the globular head and the stalk or stem. The globular head is composed of part of HA1 while the stalk or stem structure is composed of the N and C-terminal fragments of HA1 and all of HA2 (Hai et al, J. Virol, 2012 86(10): 5774-5781). The transmembrane domain and cytosolic tail are also part of HA2. The HA globular head is the main target for antibodies against influenza virus but is also highly variable and subject to constant antigenic drift. In contrast, the HA stem is highly conserved and experiences little antigenic drift but is not very immunogenic.

There remains a need for an influenza vaccine that is not restricted by inherent strain specificity, and that could provide protection against heterologous strains of influenza. There is also a need for influenza vaccines that do not require the existing egg-based production methods. In particular, it would be of great interest to have a vaccine that protects against an array of influenza strains that includes recent and evolving seasonal strains, and possible seasonal influenza strains of the future. Various approaches have been taken in attempting to provide a “universal” influenza A vaccine that protects individuals from heterologous strains, for example more recently using the conserved stem portion of HA (Yassine et al, Nature Medicine, 2015, 21(9): 1065-1070; Corbett et al, mBio, 2019, 10(1) 10:e02810-18).

### SUMMARY OF THE INVENTION

It has been found that by making amino acid substitutions in the coiled coil region of HA from influenza A strain, a stable, trimeric influenza A strain recombinant HA antigen can be obtained which retains the antigenicity of the wild type influenza strain and may be further capable of eliciting an immune response against a number of different influenza A strains.

It has further been found that certain recombinant influenza A strain HA ectodomain constructs expressed as a fusion with a heterologous trimerization domain are surprisingly stable following removal of the trimerization domain. It has also been found that certain recombinant influenza A strain HA ectodomain constructs form stable trimers which can be expressed in the absence of a trimerization domain. These stable, trimeric recombinant HA ectodomain antigens without a trimerization domain (either removed or absent) can potentially be employed in an immunogenic composition.

In one aspect there is provided an immunogenic composition comprising a recombinant influenza A strain haemagglutinin (HA) antigen in trimeric form, the antigen comprising an ectodomain of HA, without a transmembrane or cytosolic domain, wherein the ectodomain comprises:
(i) a globular head domain; and
(ii) a stem domain having a coiled coil region, comprising one or more mutations in the coiled coil region that individually or together stabilise the HA ectodomain in trimeric prefusion form;
   and wherein the recombinant HA optionally comprises a heterologous trimerization domain; together with a pharmaceutically acceptable carrier.

In another aspect there is provided an immunogenic composition comprising an isolated polynucleotide such as DNA or mRNA, encoding the recombinant HA antigen described herein, and a pharmaceutically acceptable carrier.

In another aspect there is provided the immunogenic composition described herein for use in prevention of and/or vaccination against influenza A strain infection or disease.

In another aspect there is provided the immunogenic composition for use in the prevention of and/or vaccination against influenza infection or disease caused by at least one different influenza A strain, which may be a strain from the same or a different A strain subtype from the HA subtype from which the HA ectodomain antigen is derived.

In another aspect there is provided a process for preparing an immunogenic composition described herein, the method comprising:
(i) expressing the recombinant HA antigen, from a polynucleotide sequence encoding the HA antigen fused to a heterologous trimerization domain e.g. foldon, in a eukaryotic cell;
(ii) purifying recombinant HA trimers, from the cell supernatant;
(iii) removing the trimerization domain;
(iv) combining the recombinant HA trimers with a pharmaceutically acceptable carrier.

In another aspect there is provided a process for preparing an immunogenic composition described herein comprising an HA ectodomain comprising one or more mutations in the coiled coil region that individually or together stabilise the HA ectodomain in trimeric prefusion form, the method comprising:
(i) expressing recombinant HA antigen, from a polynucleotide sequence encoding it, with or without a trimerization domain;
(ii) purifying trimeric recombinant HA, from the cell supernatant;
(iii) optionally removing the trimerization domain if present;
(iv) combining the recombinant HA trimers with a pharmaceutically acceptable carrier.

In another aspect there is provided a method for prevention of and/or vaccination against influenza A strain infection or disease, comprising the administration of an antigen or polynucleotide or immunogenic composition as described above to a person in need thereof, such as a person identified as being at risk of influenza virus infection or disease.

In another aspect there is provided a method of generating an immune response against influenza A strain, comprising administering a recombinant influenza A strain HA antigen or polynucleotide or immunogenic composition described herein, to a human subject.

In another embodiment there is provided the use of a recombinant influenza A strain HA antigen or polynucleotide described herein, in the manufacture of an immunogenic composition for generating an immune response against influenza A strain in a human subject.

In another aspect there is provided an immunogenic composition comprising a recombinant influenza A strain HA ectodomain antigen obtained by expressing an influenza A strain HA ectodomain fused to a trimerization domain followed by subsequent removal of the trimerization domain.

### BRIEF DESCRIPTION OF THE SEQUENCES

SEQ ID NO: 1 Full length HA sequence from A/Brisbane/02/2018 (H1N1)pdm09-like virus (H1_{Bri18}) also referred to as Bri18.
SEQ ID NO: 2 Full length HA sequence from A/Darwin/9/2021 H3N2 also referred to as A/Darw21 or H3 Darw21 or Dar21.
SEQ ID NO: 3 Mut10 amino acid sequence—H1 Brisbane 18 wild type sequence with mutations shown in Table 1. Includes signal sequence, foldon and poly H tail.
SEQ ID NO: 4 Mut17 amino acid sequence—H1 Brisbane 18 wild type sequence with mutations shown in Table 1. Includes signal sequence, foldon and poly H tail.
SEQ ID NO: 5 Mut18 amino acid sequence—H1 Brisbane 18 wild type sequence with mutations shown in Table 1. Includes signal sequence, foldon and poly H tail.
SEQ ID NO: 6 Mut23 amino acid sequence—H1 Brisbane 18 wild type sequence with mutations shown in Table 1. Includes signal sequence, foldon and poly H tail.
SEQ ID NO: 7 Mut24 amino acid sequence—H1 Brisbane 18 wild type sequence with mutations shown in Table 1. Includes signal sequence, foldon and poly H tail.
SEQ ID NO: 8 Mut27 amino acid sequence—H1 Brisbane 18 wild type sequence with mutations shown in Table 1. Includes signal sequence, foldon and poly H tail.
SEQ ID NO: 9 Foldon sequence
SEQ ID NO: 10 Signal peptide from H1 Brisbane 18
SEQ ID NO: 11 Nucleotide sequence encoding Mut10
SEQ ID NO: 12 Nucleotide sequence encoding Mut17
SEQ ID NO: 13 Nucleotide sequence encoding Mut18
SEQ ID NO: 14 Nucleotide sequence encoding Mut23
SEQ ID NO: 15 Nucleotide sequence encoding Mut24
SEQ ID NO: 16 Nucleotide sequence encoding Mut27
SEQ ID NO: 17 Flu622 amino acid sequence—H3 Darw21 wild type sequence with mutations shown in Table 3. Includes signal sequence, foldon and poly H tail.
SEQ ID NO: 18 Flu629 amino acid sequence—H3 Darw21 wild type sequence with mutations shown in Table 3. Includes signal sequence, foldon and poly H tail.
SEQ ID NO: 19 Flu632 amino acid sequence—H3 Darw21 wild type sequence with mutations shown in Table 3. Includes signal sequence, foldon and poly H tail.
SEQ ID NO: 20 Flu638 amino acid sequence—H3 Darw21 wild type sequence with mutations shown in Table 3. Includes signal sequence, foldon and poly H tail.
SEQ ID NO: 21 Flu639 amino acid sequence—H3 Darw21 wild type sequence with mutations shown in Table 3. Includes signal sequence, foldon and poly H tail.
SEQ ID NO: 22 Flu643 amino acid sequence—H3 Darw21 wild type sequence with mutations shown in Table 3. Includes signal sequence, foldon and poly H tail.
SEQ ID NO: 23 Flu650 amino acid sequence—H3 Darw21 wild type sequence with mutations shown in Table 3. Includes signal sequence, foldon and poly H tail.
SEQ ID NO: 24 Flu672 amino acid sequence—H3 Darw21 wild type sequence with mutations shown in Table 3. Includes signal sequence, foldon and poly H tail.
SEQ ID NO: 25 Flu679 amino acid sequence—H3 Darw21 wild type sequence with mutations shown in Table 3. Includes signal sequence, foldon and poly H tail.
SEQ ID NO: 26 Flu680 amino acid sequence—H3 Darw21 wild type sequence with mutations shown in Table 3. Includes signal sequence, foldon and poly H tail.
SEQ ID NO: 27 Flu681 amino acid sequence—H3 Darw21 wild type sequence with mutations shown in Table 3. Includes signal sequence, foldon and poly H tail.
SEQ ID NO: 28 Flu682 amino acid sequence—H3 Darw21 wild type sequence with mutations shown in Table 3. Includes signal sequence, foldon and poly H tail.
SEQ ID NO: 29 Flu683 amino acid sequence—H3 Darw21 wild type sequence with mutations shown in Table 3. Includes signal sequence, foldon and poly H tail.
SEQ ID NO: 30 Flu685 amino acid sequence—H3 Darw21 wild type sequence with mutations shown in Table 3. Includes signal sequence, foldon and poly H tail.
SEQ ID NO: 31 Flu686 amino acid sequence—H3 Darw21 wild type sequence with mutations shown in Table 3. Includes signal sequence, foldon and poly H tail.
SEQ ID NO: 32 Flu687 amino acid sequence—H3 Darw21 wild type sequence with mutations shown in Table 3. Includes signal sequence, foldon and poly H tail.
SEQ ID NO: 33 Flu688 amino acid sequence—H3 Darw21 wild type sequence with mutations shown in Table 3. Includes signal sequence, foldon and poly H tail.
SEQ ID NO: 34 Flu689 amino acid sequence—H3 Darw21 wild type sequence with mutations shown in Table 3. Includes signal sequence, foldon and poly H tail.
SEQ ID NO: 35 Flu690 amino acid sequence—H3 Darw21 wild type sequence with mutations shown in Table 3. Includes signal sequence, foldon and poly H tail.
SEQ ID NO: 36 Flu691 amino acid sequence—H3 Darw21 wild type sequence with mutations shown in Table 3. Includes signal sequence, foldon and poly H tail.
SEQ ID NO: 37 Flu692 amino acid sequence—H3 Darw21 wild type sequence with mutations shown in Table 3. Includes signal sequence, foldon and poly H tail.
SEQ ID NO: 38 Flu693 amino acid sequence—H3 Darw21 wild type sequence with mutations shown in Table 3. Includes signal sequence, foldon and poly H tail.
SEQ ID NO: 39 Flu695 amino acid sequence—H3 Darw21 wild type sequence with mutations shown in Table 3. Includes signal sequence, foldon and poly H tail.
SEQ ID NO: 40 Flu696 amino acid sequence—H3 Darw21 wild type sequence with mutations shown in Table 3. Includes signal sequence, foldon and poly H tail.
SEQ ID NO: 41 Flu697 amino acid sequence—H3 Darw21 wild type sequence with mutations shown in Table 3. Includes signal sequence, foldon and poly H tail.
SEQ ID NO: 42 Flu707 amino acid sequence—H3 Darw21 wild type sequence with mutations shown in Table 3. Includes signal sequence, foldon and poly H tail.
SEQ ID NO: 43 Signal peptide from H3 Darw21
SEQ ID NO: 44 Nucleotide sequence encoding Flu622
SEQ ID NO: 45 Nucleotide sequence encoding Flu629
SEQ ID NO: 46 Nucleotide sequence encoding Flu632
SEQ ID NO: 47 Nucleotide sequence encoding Flu638
SEQ ID NO: 48 Nucleotide sequence encoding Flu639
SEQ ID NO: 49 Nucleotide sequence encoding Flu643
SEQ ID NO: 50 Nucleotide sequence encoding Flu650
SEQ ID NO: 51 Nucleotide sequence encoding Flu672
SEQ ID NO: 52 Nucleotide sequence encoding Flu679
SEQ ID NO: 53 Nucleotide sequence encoding Flu680
SEQ ID NO: 54 Nucleotide sequence encoding Flu681
SEQ ID NO: 55 Nucleotide sequence encoding Flu682
SEQ ID NO: 56 Nucleotide sequence encoding Flu683
SEQ ID NO: 57 Nucleotide sequence encoding Flu685
SEQ ID NO: 58 Nucleotide sequence encoding Flu686
SEQ ID NO: 59 Nucleotide sequence encoding Flu687
SEQ ID NO: 60 Nucleotide sequence encoding Flu688
SEQ ID NO: 61 Nucleotide sequence encoding Flu689
SEQ ID NO: 62 Nucleotide sequence encoding Flu690
SEQ ID NO: 63 Nucleotide sequence encoding Flu691
SEQ ID NO: 64 Nucleotide sequence encoding Flu692
SEQ ID NO: 65 Nucleotide sequence encoding Flu693
SEQ ID NO: 66 Nucleotide sequence encoding Flu695
SEQ ID NO: 67 Nucleotide sequence encoding Flu696
SEQ ID NO: 68 Nucleotide sequence encoding Flu697
SEQ ID NO: 69 Nucleotide sequence encoding Flu707
SEQ ID NO: 70 H1 (Bri18) wild type—foldon polypeptide sequence: Signal sequence—HA-TEV cleavage site-foldon-His tag
SEQ ID NO: 71 H3 (Darw21) wild type—foldon polypeptide sequence: Signal sequence—HA-TEV cleavage site—foldon-His tag
SEQ ID NO: 72 HA sequence from Bri18 shown in FIG. 2, which excludes native linker and transmembrane and cytosolic regions present in full length sequence
SEQ ID NO: 73 HA sequence from Darw21 shown in FIG. 2, which excludes native linker and transmembrane and cytosolic regions present in full length sequence

### BRIEF DESCRIPTION OF THE FIGURES

FIG. 1: Full length sequence of the wild type HA polypeptide for A/Brisbane/02/2018 (H1N1)pdm09-like virus (H1_{Bri18}) also referred to herein as Bri18, showing in order: signal peptide (absent in mature protein)-HA1 chain-HA1/HA2 cleavage site-HA2 chain-region removed in constructs described herein inside dotted box, including transmembrane domain (dark background) and cytoplasmic domain. Locations of single amino acids mutated in various combinations in certain constructs described herein are shown with shading, in both HA1 and HA2 chain.

FIG. 2: Sequence comparison showing ectodomain HA sequence of H1 strain Bri18 alongside H3 strain H3-Darw21, with location of the mutations (amino acid substitutions) shown by the triangle/asterisk/cross symbols and regions A, B and C identified by different underlining.

FIG. 3: H1/H3 Wild type HA ectodomain—foldon polypeptide sequences showing linkers, TEV cleavage site, foldon and poly His tail; (a) H1 (Bri18) Wild type HA ectodomain—foldon sequence and (b) H3 (Darw21) Wild type HA ectodomain—foldon sequence.

FIG. 4: Schematic diagram of H1 and H3 wild type and recombinant HA showing in order:
signal peptide—stem, head, stem of HA1-stem of HA2-linker (native)—transmembrane domain—cytosolic tail (wild type); and
signal peptide—stem, head, stem of HA1-stem of HA2-linker (recombinant)-TEV cleavage site-linker (recombinant)-foldon-His tag (recombinant).

FIG. 5: Ribbon structure of H1 and H3 HA ectodomains (Bri18 and Darw21 respectively) shown as monomers separated out from the trimeric molecule in order to indicate locations of mutations in regions A and B of coiled coil region of stem domain. Locations of helix A and helix B are shown in H1 monomer and H3 monomer respectively; both helices are present in each monomers in the same location.

FIG. 6: Ribbon structure of H1 and H3 HA ectodomain trimers (Bri18 and Darw21) showing head and stem domains, including regions A, B and C of stem domain.

FIG. 7: Anti-HA functional HI responses induced by HA mut 10 and/23 against homologous and post-pandemic heterologous H1N1 strains at 14 days post dose 2

FIG. 8: Anti-HA IgG Antibodies and functional antibody responses induced by HA mut 10 and/23 against heterologous H1N1 strains at 14 days post dose 2

FIG. 9: Anti-Hi-stem specific CD4 and CD8 T cell responses induced by HA mut 10 and/23 at 14 days post dose 2

FIG. 10: Anti-HA IgG Antibody responses induced by HA mut 10 and/23 against pre-pandemic heterologous H1N1 and heterosubtypic (H2N2, H5N1 and H9N2) strains at 14 days post dose 2

FIG. 11: Anti-HA functional HI responses induced by HA mut 10 and/23 against pre-pandemic heterologous H1N1 and heterosubtypic (H2N2, H5N1 and H9N2) strains at 14 days post dose 2

FIG. 12: Neutralizing titers induced by HA mut 10 and/23 against heterosubtypic (H2N2, H5N1 and H9N2) strains at 14 days post dose 2

FIG. 13: Anti-HA IgG Antibody responses induced by HA mut 10 and/23 against A2 group (H3N2, H10-stem) or B lineages (B/Yam and B/Vic) strains at 14 days post dose 2

### DETAILED DESCRIPTION

### Influenza HA Ectodomain

The recombinant influenza A HA antigen provided herein comprises an HA ectodomain comprising both a head domain and a stem domain of HA. The HA antigen is thus not a stem only (or headless HA stem) antigen. The recombinant influenza HA antigen has advantageous properties such as providing a soluble HA composition. HA lacking the transmembrane domain is not inserted into a membrane and is thus soluble and not membrane-bound. Conversely, for nucleic acid delivery, the recombinant HA antigen can be linked to a transmembrane domain thus providing an antigen in vivo that does insert into the membrane.

Influenza HA is a homotrimeric surface glycoprotein, with each monomer consisting of two disulfide-linked subunits HA1 and HA2, resulting from the proteolytic cleavage products of a single HA precursor protein HA0. HA1 comprises all residues that are N-terminal to the HA1/HA2 cleavage peptide of the precursor HA0 protein and includes the receptor binding domain of the HA protein.

The HA2 chain comprises all residues that are C-terminal to the HA1/HA2 cleavage peptide of the precursor HA0 protein, including the hydrophobic peptide responsible for insertion within the host cell membrane during the process of membrane fusion, the transmembrane domain which spans the viral membrane, and cytosolic tail. For H1 strain Bri18 for example, HA1 refers to the region of the HA protein including amino acid residues from approximately 1-344 of the HA0 protein, and HA2 refers to the region of the HA protein including amino acid residues from approximately 345-566 of the HA0 polypeptide. Residues within the HA2 chain are commonly numbered independently of those in HA1, when referring to the chains independently, such that HA2 residues may be numbered e.g. 1-174 for Bri18.

The HA head domain is the globular head region of the HA protein, excluding the stem, the transmembrane domain and cytosolic region. The HA head is formed from a section of around 250-300 amino acid residues in the centre of the HA1 sequence. The HA head is comprised of the receptor binding domain and the vestigial esterase domain. It contains a sialic acid binding pocket that mediates virus attachment to the host cell.

The influenza HA stem domain is located in the membrane-proximal region of the native HA protein, directly beneath the vestigial esterase domain of the HA1 globular head. The influenza HA stem is comprised of amino acid residues from both ends of the HA1 chain as well as the ectodomain part of the HA2 chain. For H1 strain Bri18 for example, the HA stem includes residues from approximately 18-58 and 293-344 of the HA1 chain as well as residues 1-222 (or 1-176 for only the ectodomain portion) of the HA2 chain. The stem domain does not include the transmembrane or cytosolic domains.

In one embodiment, the head and stem domains are from the same influenza strain.

In one embodiment, the recombinant HA ectodomain antigen is a homotrimer, that is a trimer formed from three identical HA ectodomain monomers.

In one embodiment, the recombinant HA antigen further comprises a heterologous trimerization domain which may be fused, e.g. covalently linked, to the C-terminus. In another embodiment, the recombinant HA antigen is a trimeric antigen from which a trimerization domain, such as a heterologous trimerization domain, has been removed. In another embodiment, the recombinant HA antigen is expressed in the absence of a trimerization domain.

In one embodiment, the C-terminus of the stem domain is:
(1) covalently linked to a heterologous trimerization domain; or
(2) covalently linked to a carrier protein or a nanoparticle; or
(3) not covalently linked to another amino acid molecule.

In one embodiment, the ectodomain of the recombinant HA comprises all or substantially all of the globular head domain. By substantially all of the globular head domain is meant at least 75% or at least 85% or at least 95% or at least 99% of the length of amino acid sequence present in the wild type influenza HA head domain.

In one embodiment, the ectodomain of the recombinant HA comprises all or substantially all of the stem domain. By substantially all of the stem domain is meant at least 75% or at least 85% or at least 95% or at least 99% of the length of amino acid sequence present in the wild type influenza HA stem domain.

In one embodiment the ectodomain of the recombinant HA comprises all or substantially all, such as at least 75% or at least 85% or at least 95% or at least 99% of the amino acid sequence present in wild type influenza HA head domain, and all or substantially all, such as at least 75% or at least 85% or at least 95% or at least 99% of the amino acid sequence present in wild type influenza HA stem domain.

In one embodiment the ectodomain of the recombinant HA comprises all or substantially all, such as at least 75% or at least 85% or at least 95% or at least 99% of the amino acid sequence present in wild type influenza HA ectodomain.

In one embodiment, the recombinant HA antigen comprises amino acid residues 1-520 of influenza A HA, or an immunogenic fragment or derivative thereof having both a head and a stem domain.

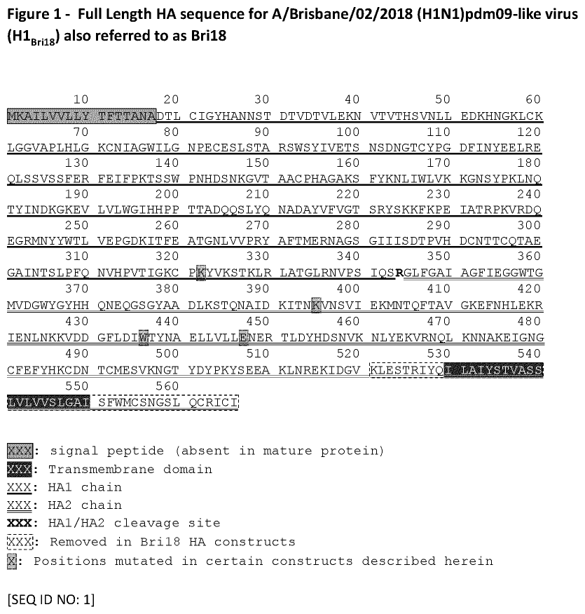

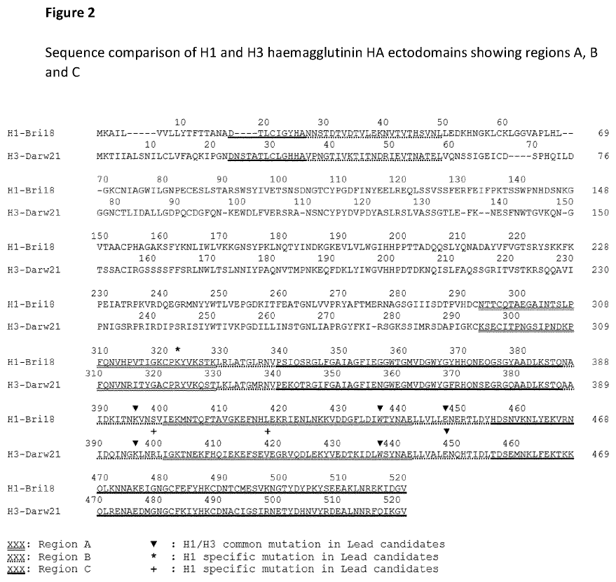

The full-length sequence of the HA polypeptide from H1 influenza strain A/Brisbane/02/2018 (H1N1)pdm09-like virus (H1_{Bri18}), also referred to herein as Bri18, is used herein as a reference sequence. This sequence is shown in FIG. 1 and SEQ ID NO: 1. The full-length sequence of the HA polypeptide from H3 influenza strain A/Darwin/9/2021 H3N2, also referred to herein as Darw21, is also used herein as a reference sequence. This sequence is shown in the sequence comparison with Bri18 in FIG. 2 (ectodomain) and in SEQ ID NO: 2 (full-length). The specific amino acid sequences and locations referred to herein for influenza A strain HA relate to these reference sequences. However it will be evident that for other influenza A isolates and sequences, in particular those from different subtypes, where the numbering and/or amino acids at specific positions may differ, the equivalent sequences and locations in those other isolates and sequences are also included within the scope of the polypeptide and polynucleotide constructs described herein. These equivalent sequences and locations in other influenza A isolates will be evident to the person skilled in the art, from the description and figures provided herein.

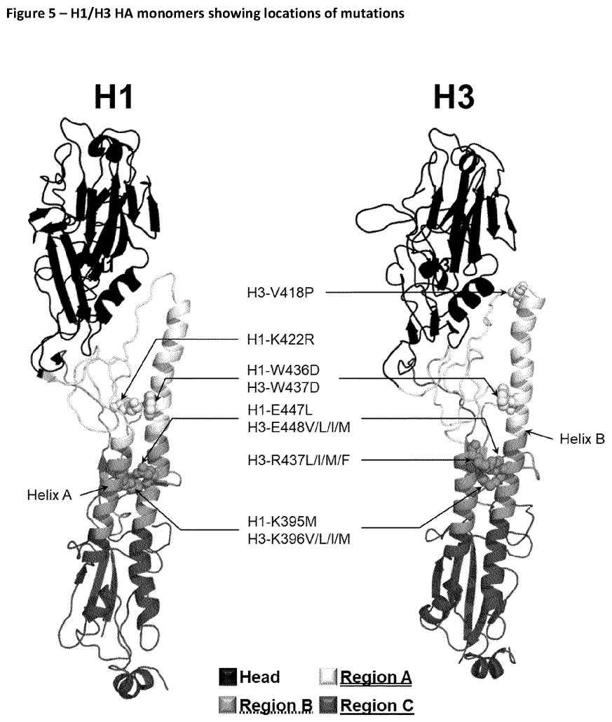

The two strains illustrated here are from two different subtypes H1 and H3, which subtypes are from the two different groups (or clades) of influenza A known as group 1 and group 2, also referred to as group A1 and group A2. The full length Bri18 HA sequence shown in FIG. 1 and SEQ ID NO: 1 includes the signal sequence, HA1, HA2, transmembrane and cytosolic regions. In the literature, numbering for HA commonly uses H3 influenza as reference sequence, however this convention is not followed here because the starting point in the present case is HA sequences from H1 strains of influenza. It will be evident that equivalent locations in HA ectodomains from other strains can be located by reference to sequence comparisons and/or structural comparisons. For example, FIG. 2 shows a sequence alignment for HA ectodomains from strain Bri18, which is an H1 strain from group 1 influenza A, alongside HA ectodomain from Darw21, which is an H3 strain from group 2 influenza A, including positions of certain stabilising mutations in the two sequences. FIG. 5 shows a structural comparison in the form of ribbon diagrams for a monomer of each of the HA trimers from the same two H1 and H3 strains, indicating the locations of equivalent stabilising mutations described herein for the HAs from these two strains. Structural comparisons may be carried out in 2-D form as shown in FIG. 5, or in 3-D form, or both.

In one embodiment, the HA ectodomain comprises all of the HA1, and all of the HA2 of influenza HA without the transmembrane and cytosolic domains, or an immunogenic fragment or derivative thereof having both a head and a stem domain. In a particular embodiment, the HA ectodomain comprises amino acid residues 1-520 of influenza A HA from Bri18 or an equivalent sequence comprising all of the HA1 and all of the HA2 from another influenza A strain HA, or an immunogenic fragment or derivative thereof having both a head and a stem domain.

The recombinant influenza A strain HA antigens described herein have been found to have a number of useful properties. One advantage relates to the nature of the ectodomain antigen which possesses antigenic properties of the HA stem, while also retaining antigenic properties of the HA globular head. Furthermore, mutations which stabilise the coiled coil of the recombinant HA antigen can in certain cases allow for removal of a trimerization domain used in the production of the recombinant HA (as shown herein for H1), or potentially allow for expression of the recombinant antigen in the absence of a trimerization domain (e.g. for H3). Due to the absence of the HA transmembrane region and the cytosolic region, the recombinant ectodomain when expressed is soluble and readily purified. Stabilising mutations identified herein improve the manufacturability attributes of recombinant HA, such as yield and thermostability. Furthermore, by stabilising the structure of the trimer in the native trimeric prefusion state, the antigenic properties of HA are optimised for generating an immune response that recognises wild type influenza virus when encountered in vivo. This immune response has been shown to be directed not only against the influenza strain from which the HA of the recombinant antigen originates, but also against other influenza strains, including strains from different influenza A subtypes.

In one embodiment, the influenza HA antigens described herein are capable of binding to antibodies directed to at least one epitope on wild type HA, suitably a neutralising epitope. Thus, modifications to the wild type influenza HA in the antigens described herein preserve the conformation of at least one wild type HA epitope, suitably a neutralising epitope. In one embodiment, the recombinant influenza HA antigen retains one or more epitopes present in the wild type, suitably the CR9114 or the Fl6, or more suitably both the CR9114 and the Fl6 epitopes.

The recombinant influenza A HA antigen described herein contains mutations such as amino acid deletions, substitutions (e.g. single amino acid substitutions) or additions compared to the wild type HA amino acid sequence. For the purposes herein, substitution refers to replacement of the wild type amino acid with any other amino acid at that same amino acid position. In one embodiment, the one or more mutations stabilise the antigen in the correct conformation, such that it is capable of eliciting an immune response against native HA. In one embodiment the one or more mutations stabilise the antigen in trimeric form. In one embodiment, the one or more mutations stabilise the antigen in prefusion form.

Typically, the recombinant influenza HA antigen described herein contains a number of amino acid substitutions (e.g. single amino acid substitutions) such as up to 4 or up to 6 or up to 8 or up to 10 amino acid substitutions, e.g. between 1 and 4 or 1 and 6 amino acid substitutions, e.g. 1, 2, 3, 4, 5 or 6 amino acid substitutions compared to native HA. Typically the amino acid substitutions (e.g. single amino acid substitutions) are in HA2. There may also be one or more amino acid substitutions in HA1, in particular one amino acid substitution in HA1. In one embodiment there is one amino acid substitution in HA1 and one or two or three or four amino acid substitutions in HA2.

In one embodiment, the recombinant HA ectodomain antigen does not comprise additional elements of HA such as the transmembrane region or cytosolic region. In one embodiment, the HA antigen is not membrane bound, due to the absence of the transmembrane region, or the absence of both the transmembrane and cytosolic regions. In an alternative embodiment for nucleic acid delivery, the HA antigen delivered by a nucleic acid delivery platform such as mRNA comprises a transmembrane region such as an HA transmembrane region, with or without the cytosolic region. The transmembrane region of influenza A strain HA is located from around amino acid positions 530-550 and the cytosolic tail from around positions 551-566, for the H1 HA sequence shown herein. See schematic diagram in FIG. 4.

### Prefusion Conformation

The influenza HA antigen is suitably stabilised in the prefusion conformation or state. Stabilisation is achieved by virtue of stabilising mutations in the coiled coil region which stabilise the HA trimer, such as one or more amino acid substitutions e.g. single amino acid substitutions. Stabilisation may be additionally aided by the presence of a trimerization domain. Mutations in the coiled coil region, such as one or more amino acid substitutions e.g. single amino acid substitutions, can potentially stabilise the HA once the trimerization domain has been removed following expression of the recombinant HA antigen, for example by enzymatic cleavage. Thus, in one embodiment, the HA antigen is expressed with a trimerization domain which is removed following expression. In a particular embodiment, the HA antigen is an ectodomain antigen without a trimerization domain, more particularly an ectodomain antigen expressed with a trimerization domain and from which the trimerization domain has been cleaved, e.g. enzymatically, following expression.

Stabilisation may be effected for example by helix stabilization, loop optimization, disulphide bond addition, and side-chain repacking. Stabilisation of HA may be achieved by introducing mutations (e.g. amino acid substitutions such as single amino acid substitutions) that form or strengthen ionic bonds, salt bridges, or that increase hydrophobic packing or cavity filling. Hydrophobic packing promotes and/or drives the association of hydrophobic regions together to exclude water. Cavity filling fills an unoccupied cavity volume found either buried inside (i.e. inside the monomer) or at the interface of (i.e. between the monomers in the trimer) the haemagglutinin protein by introduction of an amino acid (e.g. an amino acid substitution such as a single amino acid substitution) that fills such a space and allows for and/or promotes good folding/packing and avoids the tendency for water to become encapsulated or incorporated into the folding of the protein. This may be achieved for example by replacing an amino acid with a small side chain such as (but not limited to) serine, with an amino acid with a larger side chain. Stabilisation of homotrimeric HA antigen may be assessed by characterisation studies such as those described in the Examples. In one embodiment, stabilisation in prefusion form is assessed by determining the presence of trimeric HA. Alternatively or additionally, stabilisation in prefusion form is assessed by determining the presence of epitopes such as the CR9114 and/or Fl6 epitopes (e.g. Fl6v3), e.g. by means of a mAb binding assay.

The prefusion conformation of HA is discussed in the literature for example Wu & Wilson, Viruses, 2020, 12: 1053, and Ni et al 2014. Stabilising mutations are discussed in the literature for example in Yassine et al, Nature Medicine, 2015, 21(9): 1065-1070.

It will be understood that additional alterations may be present compared to wild type HA, in either the head and or the stem regions or both, that will either not negatively affect, or that will further optimise, the recombinant influenza HA described herein. For example, amino acid insertions, deletions or substitutions may be made that do not disrupt the prefusion conformation of the HA or negatively impact the antigenic properties of the HA described herein. Such amino acid substitutions, deletions or insertions may be for example, for altering the properties of one or more epitopes of the HA either in the globular head or in the stem region, or both.

### Stabilising Mutations

Stabilisation may be determined by looking at one or more different parameters following expression and purification, such as productivity (yield) of expression of the recombinant antigen relative to productivity of the wild type antigen when it is expressed recombinantly. Higher productivity is often associated with more stable folding of a recombinant protein. Alternatively or additionally, structural analyses may be performed such as nanoDSF and/or stress tests, to confirm improved folding stability. Stress tests include for example thermostability testing, such as evaluating the level of degradation and/or aggregation of the recombinant antigen after a week at room temperature or 37° C. Alternatively or additionally, stabilisation may be assessed by presence of trimers and/or by examining presence of epitopes present on the native HA such as the CR9114 and the Fl6 (such as Fl6v3) epitopes. Suitably, a stabilised HA antigen will be equal to or improved over wild type recombinant HA in relation to at least one parameter, suitably two or more parameters, where such parameters are selected from the parameters described herein such as yield following expression, folding stability as measured by nanoDSF, stress tests such as thermostability testing, presence of trimers, and presence of the CR9114 and/orFl6 (such as Fl6v3) epitopes as measured by binding to CR9114 and/or Fl6 (such as Fl6v3) antibodies.

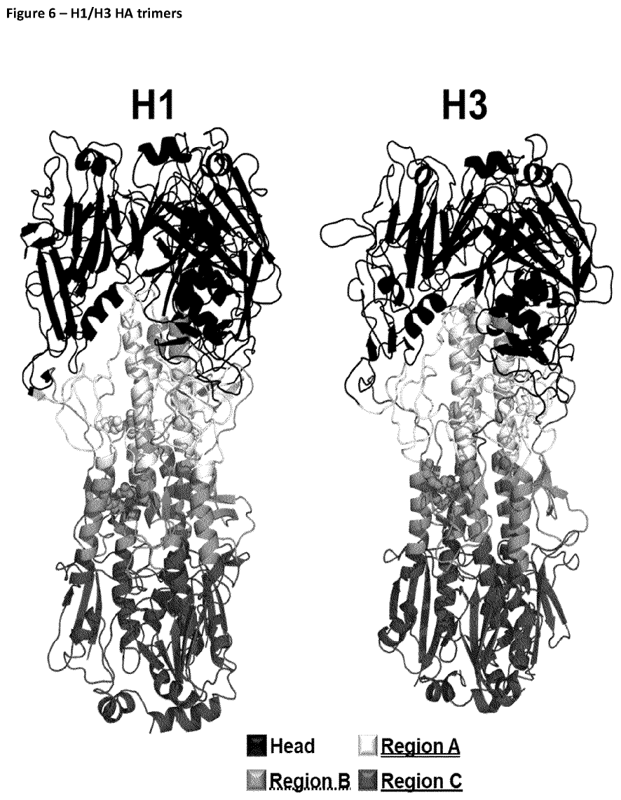

The influenza HA trimeric antigen described herein may be stabilised by mutations introduced into the coiled coil region, suitably site-specific mutations for example individual amino acid substitutions, additions or deletions designed to confer improved stability on the HA antigen or trimer. These may be mutations in the coiled coil core or in the region immediately surrounding the coiled coil core. In one embodiment, the HA antigen comprises one or more stabilising mutations in a helix structure of the coiled coil for example helix A or helix B of prefusion influenza HA, see FIG. 5, in which helix A is the smaller helix and helix B is the larger helix in each monomer. In another embodiment, the HA antigen comprises one or more stabilising mutations in one or more of Regions A, B and C. Regions A, B and C are illustrated in FIGS. 2, 4, 5 and 6 in relation to an H1 and an H3 strain.

In one embodiment, the coiled coil region of the stem domain comprising one or more mutations is from 317 to 472, such as from 322 to 467 for H1; or from 342 to 473, such as from 347 to 468 for H2; or equivalent ranges for the coiled coil region in other HA A strains or subtypes.

For example, it has been found that a mutation (e.g. a single amino acid substitution) at one or more of positions 322, 395, 431, 432, 436, 438, 439, 447, 449, 450, 453, 460, 464 and 467 of HA from an H1 subtype, more particularly strain Bri18, can help to stabilise the HA ectodomain. Similarly, it has been found that a mutation (e.g. a single amino acid substitution) at one or more of positions 347, 396, 399, 418, 428, 437, 440, 448, 451, 454, 465 and 468 from an H3 subtype, more particularly strain Darw21, can help to stabilise the HA ectodomain. In particular, it has been found that an amino acid substitution at position 395, and optionally 322, 436 and 447 as well, can have a beneficial effect on a recombinant HA from an H1 strain for example an improvement in yield.

Similarly, it has been found that an amino acid substitution at a position selected from one or more of 396, 399, 418, 437 and 448 can have a beneficial effect on a recombinant HA from an H3 strain.

For example, it has been found that a mutation (e.g., a single amino acid substitution) at position 395 of HA from the H1 subtype, in particular a K395M substitution (example H1 construct from strain Bri18 herein referred to as Mut10), or a mutation (e.g. a single amino acid substitution) at position 396 of HA from the H3 subtype, in particular a K396V/L/I/M substitution, helps to stabilise the ectodomain trimer. This mutation (395/396 depending on the strain) is in a smaller alpha helix adjacent to the coiled coil, referred to herein as helix A (see smaller helix in FIG. 5 depicting monomers of HA). It has also been found that a K395M substitution plus three other substitutions: K322R, W436D and E447L in subtype H1 (example herein referred to as Mut23) provide a beneficial effect, on protein yield in particular. Exemplary H1 HA ectodomain antigens Mut10 and Mut23 described herein have been shown to stimulate broad anti-HA responses to different influenza strains within the H1 subtype and to different influenza strains from other subtypes within group 1, such as H2, H5 and H9.

Similarly, a combination of substitutions at some of the equivalent positions in H3: K396V/L/I/M, W437D and E448V/L/1/M with a further substitution R399L/I/M/F, stabilise the ectodomain for this subtype.

In one embodiment, the influenza HA antigen comprises one or more, two or more, three or more, four or more, five or more, six or more, seven or more, eight or more, nine or more, ten or more, eleven or more, twelve or more, thirteen or more, or fourteen of the following amino acid substitutions compared to wild type HA: K322R, K395M, G431C, F432C, W436D, Y438D, N439L, E447L, E449Q, R450W, D453L, K4601, E464F and R467M, in particular one or more, two or more, three or more, four or more, five or more, six or more, seven or more, eight or more, or nine of the following amino acid substitutions compared to wild type HA: K322R, K395M, W436D, N439L, E447L, E449Q, R450W, D453L and K4601. In one embodiment, the recombinant H1 HA antigen contains one or more, two or more, three or more, four or more, five or more, six or more, seven or more, or eight of the following mutations: K322R, G431C, F432C, W436D, Y438D, K4601, E464F and R467M. In certain embodiments, the HA is from H1.

In one embodiment, the recombinant HA ectodomain contains K395M and one or more, two or more, three or more, four or more, five or more, six or more, seven or more, eight or more, nine or more, ten or more, eleven or more, twelve or more, or thirteen of the following mutations (e.g., amino acid substitutions) compared to wild type HA: K322R, G431C, F432C, W436D, Y438D, N439L, E447L, E449Q, R450W, D453L, K4601, E464F and R467M. In one embodiment, the recombinant HA ectodomain contains K395M and one or more, two or more, three or more, four or more, five or more, six or more, seven or more, or eight of the following mutations (e.g. amino acid substitutions) compared to wild type: K322R, G431C, F432C, W436D, Y438D, K4601, E464F and R467M. In certain embodiments, the HA is from H1.

In certain embodiments the antigen is from H1 and comprises a mutation or combination of mutations presented in Table 1 or Table 2. Examples of further HA antigens comprising the Table 1 mutations are described in Table 2 and in the Examples, including for example the combinations listed in Table 1 below for certain HA ectodomain constructs Mut10, Mut17, Mut18, Mut23, Mu24 and Mu27.

TABLE 1

| | K322R | K395M | W436D | N439L | E447L | E449Q | R450W | D453L | K4601 |
|---|---|---|---|---|---|---|---|---|---|
| Mut10 | | ✓ | | | | | | | |
| Mut17 | | ✓ | | | ✓ | ✓ | ✓ | ✓ | |
| Mut18 | | | | | | | | | ✓ |
| Mut23 | ✓ | ✓ | ✓ | | ✓ | | | | |
| Mut24 | ✓ | | ✓ | ✓ | | ✓ | ✓ | ✓ | |
| Mut27 | ✓ | ✓ | ✓ | ✓ | ✓ | ✓ | ✓ | ✓ | |

TABLE 2

| Position | 322 | 395 | 431 | 432 | 436 | 438 | 439 | 447 | 449 | 450 | 453 | 460 | 464 | 467 |
|---|---|---|---|---|---|---|---|---|---|---|---|---|---|---|
| WT | K | K | G | F | W | Y | N | E | E | R | D | K | E | R |
| Mut1 | — | — | C | C | — | — | — | — | — | — | — | — | — | — |
| Mut2 | — | — | — | — | — | — | L | — | — | — | — | — | — | — |
| Mut3 | — | — | — | — | D | — | — | — | — | — | — | — | — | — |
| Mut4 | R | — | — | — | — | — | — | — | — | — | — | — | — | — |
| Mut5 | — | — | — | — | — | D | — | — | — | — | — | — | — | — |
| Mut6 | R | — | — | — | D | — | — | — | — | — | — | — | — | — |
| Mut7 | R | — | — | — | D | — | L | — | — | — | — | — | — | — |
| Mut8 | — | — | — | — | — | — | — | — | — | W | — | — | — | — |
| Mut9 | — | — | — | — | — | — | — | — | — | — | L | — | — | — |
| Mut10 | — | M | — | — | — | — | — | — | — | — | — | — | — | — |
| Mut11 | — | — | — | — | — | — | — | L | — | — | — | — | — | — |
| Mut12 | — | — | — | — | — | — | — | — | Q | — | — | — | — | — |
| Mut13 | — | — | — | — | — | — | — | — | Q | W | — | — | — | — |
| Mut14 | — | | — | — | — | — | — | — | — | W | L | — | — | — |
| Mut15 | — | M | — | — | — | — | — | L | — | — | — | — | — | — |
| Mut16 | — | — | — | — | — | — | — | — | Q | W | L | — | — | — |
| Mut17 | — | M | — | — | — | — | — | L | Q | W | L | — | — | — |
| Mut18 | — | — | — | — | — | — | — | — | — | — | — | I | — | — |
| Mut19 | — | — | — | — | — | — | — | — | — | — | — | — | — | M |
| Mut20 | — | — | — | — | — | — | — | — | — | — | — | I | — | M |
| Mut21 | — | — | — | — | — | — | — | — | — | — | — | I | F | M |
| Mut22 | — | — | — | — | — | — | L | — | Q | W | L | — | — | — |
| Mut23 | R | M | — | — | D | — | — | L | — | — | — | — | — | — |
| Mut24 | R | — | — | — | D | — | L | — | Q | W | L | — | — | — |
| Mut25 | — | M | — | — | — | — | L | L | Q | W | L | — | — | — |
| Mut26 | — | M | — | — | — | D | L | L | Q | W | L | — | — | — |
| Mut27 | R | M | — | — | D | — | L | L | Q | W | L | — | — | — |
| Mut28 | — | — | — | — | — | — | — | — | Q | W | L | I | F | M |
| Mut29 | — | M | — | — | — | — | — | L | — | — | — | I | F | M |
| Mut30 | — | M | — | — | — | — | — | L | Q | W | L | I | F | M |
| Mut31 | — | — | — | — | — | — | L | — | — | — | — | I | F | M |
| Mut32 | R | — | — | — | D | — | — | — | — | — | — | I | F | M |
| Mut33 | R | — | — | — | D | — | L | — | — | — | — | I | F | M |

The mutations described in Table 1 and Table 2 for H1 and similarly in Table 3 for H3, target different regions or zones of HA, which are located by reference to the annotated amino acid sequence and HA structure diagram in FIGS. 2, 4, 5 and 6:
(i) Region A which is the membrane distal fusion domain region;
(ii) Region B which is the central fusion domain region; and
(iii) Region C which is the fusion peptide/membrane proximal fusion domain region.

In one embodiment, the influenza HA antigen comprises one or more Region A mutations for example a substitution (e.g. a single amino acid substitution) at a position selected from one or more of 322/323 and 436/437.

In another embodiment, the antigen comprises one or more Region B mutations for example a substitution (e.g. a single amino acid substitution) at a position selected from one or more of 395/396 and 447/448. In a particular embodiment referencing numbering for the H3 strain Darw21 illustrated herein, the antigen comprises a combination of Region B mutations for example a substitution (e.g. a single amino acid substitution) at each of positions 396, 399 and 448 and optionally 437.

In another embodiment, the antigen comprises one or more Region C mutations for example a substitution (e.g. a single amino acid substitution) at a position in region C.

In further embodiments, the antigen comprises one or more mutations e.g. single amino acid substitutions from a combination of each of Region A and B, or each of Region A and C, or each of Region B and C, or all three regions A, B and C.

In the following embodiments, the numbering refers to influenza A strains with amino acid positions located according to the H1 sequence Bri18 illustrated herein.

In one embodiment, the recombinant HA ectodomain contains the K395M mutation in the absence of other mutations in the coiled coil region (e.g. Mut10).

In one embodiment, the recombinant HA ectodomain contains the K322R, K395M, W436D and E447L mutations in the absence of other mutations in the coiled coil region (e.g. Mut23 for H1). In one embodiment, the recombinant HA ectodomain contains the K395M mutation and one additional mutation selected from K322R, W436D and E447L, in the absence of other mutations in the coiled coil region.

In one embodiment, the recombinant HA ectodomain contains the K395M mutation and two additional mutations selected from K322R, W436D and E447L, in the absence of other mutations in the coiled coil region.

In one embodiment, the recombinant HA ectodomain contains the K322R and K395M mutations in the absence of other mutations in the coiled coil region.

In one embodiment, the recombinant HA ectodomain contains the K395M and W436D mutations in the absence of other mutations in the coiled coil region.

In one embodiment, the recombinant HA ectodomain contains the K395M and E447L mutations in the absence of other mutations in the coiled coil region.

In one embodiment, the recombinant HA ectodomain contains the K322R, K395M and W436D mutations in the absence of other mutations in the coiled coil region.

In one embodiment, the recombinant HA ectodomain contains the K322R, K395M and E447L mutations in the absence of other mutations in the coiled coil region.

In one embodiment, the recombinant HA ectodomain contains the K395M, W436D and E447L mutations in the absence of other mutations in the coiled coil region.

In the following embodiments, the numbering refers to influenza A strains with amino acid positions located according to the H3 sequence Darw21 illustrated herein.

In one embodiment, the recombinant HA ectodomain contains the V418P mutation in the absence of other mutations in the coiled coil region (e.g. Flu639 for H3).

In one embodiment, the recombinant HA ectodomain contains the V418P mutation and the W437D mutation in the absence of other mutations in the coiled coil region (e.g. Flu707 for H3).

In one embodiment, the recombinant HA ectodomain contains a K3961/L and a R399F/L and a E448L/I mutation with or without the W437D mutation, in the absence of other mutations in the coiled coil region (e.g. Flu632, Flu680, Flu689 for H3). In particular embodiments, the recombinant HA ectodomain contains the K3961, R399F and E448L mutations in the absence of other mutations in the coiled coil region (e.g. Flu632 for H3); or K396L, R399L, W437D and E4481 mutations in the absence of other mutations in the coiled coil region (e.g. Flu680 for H3); or K396L, R399F, W437D and E448L mutations in the absence of other mutations in the coiled coil region (e.g. Flu689 for H3).

In certain embodiments the antigen comprises a mutation or combination of mutations presented in Table 3. Examples of HA antigens comprising the Table 3 mutations are described in the Examples, including for example the combinations listed in Table 3 below for certain HA ectodomain constructs Flu622, Flu629, Flu632, Flu638, Flu639, Flu643, Flu650, Flu672, Flu679, Flu680, Flu681, Flu682, Flu683, Flu685, Flu686, Flu687, Flu688, Flu689, Flu690, Flu691, Flu692, Flu693, Flu695, Flu696, Flu697 and Flu707.

TABLE 3

| | 347 | 396 | 399 | 418 | 428 | 437 | 440 | 448 | 451 | 454 | 455 | 468 |
|---|---|---|---|---|---|---|---|---|---|---|---|---|
| Name | I | K | R | V | Y | W | N | E | H | D | E | K |
| Flu622 | — | L | L | — | — | — | — | M | — | — | — | — |
| Flu629 | — | L | M | — | — | — | — | I | — | — | — | — |
| Flu632 | — | I | F | — | — | — | — | L | — | — | — | — |
| Flu638 | — | V | I | — | — | — | — | M | — | — | — | — |
| Flu639 | — | — | — | P | — | — | — | — | — | — | — | — |
| Flu643 | — | — | — | — | — | — | I | — | — | — | — | — |
| Flu650 | F | — | — | — | — | — | — | — | L | A | — | — |
| Flu672 | — | — | — | — | — | — | — | — | — | — | M | M |
| Flu679 | — | L | L | — | — | D | — | L | — | — | — | — |
| Flu680 | — | L | L | — | — | D | — | I | — | — | — | — |
| Flu681 | — | L | L | — | — | D | — | M | — | — | — | — |
| Flu682 | — | V | L | — | — | D | — | L | — | — | — | — |
| Flu683 | — | V | L | — | — | D | — | I | — | — | — | — |
| Flu685 | — | M | L | — | — | D | — | V | — | — | — | — |
| Flu686 | — | M | L | — | — | D | — | L | — | — | — | — |
| Flu687 | — | M | L | — | — | D | — | I | — | — | — | — |
| Flu688 | — | L | M | — | — | D | — | I | — | — | — | — |
| Flu689 | — | L | F | — | — | D | — | L | — | — | — | — |
| Flu690 | — | L | F | — | — | D | — | I | — | — | — | — |
| Flu691 | — | I | F | — | — | D | — | L | — | — | — | — |
| Flu692 | — | L | I | — | — | D | — | L | — | — | — | — |
| Flu693 | — | L | I | — | — | D | — | I | — | — | — | — |
| Flu695 | — | V | I | — | — | D | — | L | — | — | — | — |
| Flu696 | — | V | I | — | — | D | — | I | — | — | — | — |
| Flu697 | — | V | I | — | — | D | — | M | — | — | — | — |
| Flu707 | — | — | — | P | — | D | — | — | — | — | — | — |

It will be evident that equivalent locations for substitutions and other mutations in HA ectodomains from influenza strains other than Bri18 or Darw2l, for example but not limited to other Hi strains or other H3 strains, are included within the present scope. These equivalent locations can be located by reference to sequence comparisons with the Bri18 HA or Darw21 sequence presented herein, similar to the sequence comparison illustrated in FIG. 2. Equivalent locations for stabilising mutations in HA ectodomains from other influenza strains can alternatively or additionally be identified using a structural comparison. The ribbon diagram illustrated in FIG. 5 showing the comparison of the H1 and H3 (Bri18 and Darw21) monomers in which the structural location of the stabilising mutations is indicated, is one such structural comparison. Other structural comparisons can involve 3-D comparisons. A sequence comparison may be used to determine equivalent locations, followed by confirmation using a structural comparison, or vice versa.

In one embodiment, the mutations (e.g. single amino acid substitutions) introduced into the HA ectodomain described herein do not create disulphide bonds.

The influenza HA antigen may be comprised within a construct which comprises further polypeptide sequences. The further polypeptide sequences may include, for example, one or more signal peptides. In some embodiments, the signal peptide is not present in the final construct of the HA antigen or compositions or methods or uses herein.

It will be evident that the particular mutations described herein in the Tables potentially can be switched for conservative amino acid substitutions i.e. the replacement amino acid can itself be conservatively substituted with an amino acid with similar properties that achieves similar stabilisation.

As referred to herein, conservative amino acid substitution means substitution of an amino acid residue by another amino acid residue having a side chain (R group) with similar chemical properties and will generally not substantially change the functional properties of a protein in which the substitution was made.

Examples of groups of conservative amino acids substitutions include:
(1) basic side chains: arginine, histidine, and lysine;
(2) acidic side chains: aspartate (aspartic acid) and glutamate (glutamic acid);
(3) aromatic side chains: phenylalanine, tryptophan and tyrosine;
(4) amide-containing side chains: asparagine and glutamine;
(5) sulfur-containing side chains: cysteine and methionine;
(6) aliphatic side chains: glycine, alanine, valine, leucine and isoleucine; and
(7) aliphatic-hydroxyl side chains: serine and threonine.

Examples of conservative amino acids substitutions include:
alanine-valine,
arginine-lysine,
aspartate-glutamate,
asparagine-glutamine,
isoleucine-leucine-valine, and
phenylalanine-tyrosine.

The percent sequence identity/degree of similarity may be adjusted to account for conservative substitutions (see, Pearson (1994) Methods Mol. Biol. 24: 307-331).

In particular embodiments, the HA antigen comprises or more suitably consists of a construct described in Tables 1, 2 and 3, such as a polypeptide sequence selected from SEQ ID NOS: 3 to 8 and SEQ ID NOS: 17-42 with or without the signal peptide, or a sequence having at least 85% identity or at least 87% identity or at least 90% identity, such as 95% or greater, such as 98% or greater, such as 99% or greater sequence identity to any one of the amino acid sequences of SEQ ID NOS: 3-8 and 17-42 with or without the signal peptide. In further embodiments, the HA antigen comprises a polypeptide sequence of SEQ ID NOS: 3-8 or 17-42, with or without the signal peptide, from which certain elements, such as the His tag or the His tag and the trimerization domain, are absent.

For example, the HA antigen may comprise one of the following, each of which may be without the signal peptide in its final form:
(1) an HA ectodomain antigen comprising mutations as shown for Mut10 in Table 1, suitably SEQ ID NO: 3, with or without a trimerization domain and His tag;
(2) an HA ectodomain antigen comprising mutations as shown for Mut17 in Table 1, suitably SEQ ID NO: 4, with or without a trimerization domain and His tag;
(3) an HA ectodomain antigen comprising mutations as shown for Mut18 in Table 1, suitably SEQ ID NO: 5, with or without a trimerization domain and His tag;
(4) an HA ectodomain antigen comprising mutations as shown for Mut23 in Table 1, suitably SEQ ID NO: 6, with or without a trimerization domain and His tag;
(5) an HA ectodomain antigen comprising mutations as shown for Mut24 in Table 1, suitably SEQ ID NO: 7, with or without a trimerization domain and His tag;
(6) an HA ectodomain antigen comprising mutations as shown for Mut27 in Table 1, suitably SEQ ID NO: 8, with or without a trimerization domain and His tag;
(7) an HA ectodomain antigen comprising mutations as shown for Flu622 in Table 3, suitably SEQ ID NO: 17, with or without a trimerization domain and His tag;
(8) an HA ectodomain antigen comprising mutations as shown for Flu629 in Table 3, suitably SEQ ID NO: 18, with or without a trimerization domain and His tag;
(9) an HA ectodomain antigen comprising mutations as shown for Flu632 in Table 3, suitably SEQ ID NO: 19, with or without a trimerization domain and His tag;
(10) an HA ectodomain antigen comprising mutations as shown for Flu638 in Table 3, suitably SEQ ID NO: 20, with or without a trimerization domain and His tag;
(11) an HA ectodomain antigen comprising mutations as shown for Flu639 in Table 3, suitably SEQ ID NO: 21, with or without a trimerization domain and His tag;
(12) an HA ectodomain antigen comprising mutations as shown for Flu643 in Table 3, suitably SEQ ID NO: 22, with or without a trimerization domain and His tag;
(13) an HA ectodomain antigen comprising mutations as shown for Flu650 in Table 3, suitably SEQ ID NO: 23, with or without a trimerization domain and His tag;
(14) an HA ectodomain antigen comprising mutations as shown for Flu672 in Table 3, suitably SEQ ID NO: 24, with or without a trimerization domain and His tag;
(15) an HA ectodomain antigen comprising mutations as shown for Flu679 in Table 3, suitably SEQ ID NO: 25, with or without a trimerization domain and His tag;
(16) an HA ectodomain antigen comprising mutations as shown for Flu680 in Table 3, suitably SEQ ID NO: 26, with or without a trimerization domain and His tag;
(17) an HA ectodomain antigen comprising mutations as shown for Flu681 in Table 3, suitably SEQ ID NO: 27, with or without a trimerization domain and His tag;
(18) an HA ectodomain antigen comprising mutations as shown for Flu682 in Table 3, suitably SEQ ID NO: 28, with or without a trimerization domain and His tag;
(19) an HA ectodomain antigen comprising mutations as shown for Flu683 in Table 3, suitably SEQ ID NO: 29, with or without a trimerization domain and His tag;
(20) an HA ectodomain antigen comprising mutations as shown for Flu685 in Table 3, suitably SEQ ID NO: 30, with or without a trimerization domain and His tag;
(21) an HA ectodomain antigen comprising mutations as shown for Flu686 in Table 3, suitably SEQ ID NO: 31, with or without a trimerization domain and His tag;
(22) an HA ectodomain antigen comprising mutations as shown for Flu687 in Table 3, suitably SEQ ID NO: 32, with or without a trimerization domain and His tag;
(23) an HA ectodomain antigen comprising mutations as shown for Flu688 in Table 3, suitably SEQ ID NO: 33, with or without a trimerization domain and His tag;
(24) an HA ectodomain antigen comprising mutations as shown for Flu689 in Table 3, suitably SEQ ID NO: 34, with or without a trimerization domain and His tag;
(25) an HA ectodomain antigen comprising mutations as shown for Flu690 in Table 3, suitably SEQ ID NO: 35, with or without a trimerization domain and His tag;
(26) an HA ectodomain antigen comprising mutations as shown for Flu691 in Table 3, suitably SEQ ID NO: 36, with or without a trimerization domain and His tag;
(27) an HA ectodomain antigen comprising mutations as shown for Flu692 in Table 3, suitably SEQ ID NO: 37, with or without a trimerization domain and His tag;
(28) an HA ectodomain antigen comprising mutations as shown for Flu693 in Table 3, suitably SEQ ID NO: 38, with or without a trimerization domain and His tag;
(29) an HA ectodomain antigen comprising mutations as shown for Flu695 in Table 3, suitably SEQ ID NO: 39, with or without a trimerization domain and His tag;
(30) an HA ectodomain antigen comprising mutations as shown for Flu696 in Table 3, suitably SEQ ID NO: 40, with or without a trimerization domain and His tag;
(31) an HA ectodomain antigen comprising mutations as shown for Flu697 in Table 3, suitably SEQ ID NO: 41, with or without a trimerization domain and His tag;
(32) an HA ectodomain antigen comprising mutations as shown for Flu707 in Table 3, suitably SEQ ID NO: 42, with or without a trimerization domain and His tag;

In further embodiments, specifically for nucleic acid delivery, the HA antigen comprises a polypeptide sequence of SEQ ID NOS: 3-8 and 17-42, with or without the signal peptide, or a sequence having at least 85% identity or at least 87% identity or at least 90% identity, such as 95% or greater, such as 98% or greater, such as 99% or greater sequence identity to any one of the amino acid sequences of SEQ ID NOS: 3-8 and 17-42, with or without the signal peptide, and without the trimerisation domain and without the His tag and further comprising a transmembrane domain (which may be homologous or heterologous and is optionally trimeric), and optionally a cytosolic domain. In particular embodiments for nucleic acid delivery, the HA antigen comprises a polypeptide sequence selected from (1) to (32) above, with or without the signal peptide, and without the trimerization domain or the His tag, and further comprising a transmembrane domain (homologous or heterologous) and optionally a cytosolic region. In embodiments for nucleic acid delivery, the transmembrane domain may be an HA transmembrane domain, such as the transmembrane domain from the influenza strain from which the antigen is derived i.e. a homologous transmembrane domain. The cytosolic domain may be an HA cytosolic domain, such as the cytosolic domain from the influenza strain from which the antigen is derived i.e. a homologous cytosolic domain. The purpose of the transmembrane and cytosolic domains is to function as a membrane anchor for the HA antigen.

Particles/Nanoparticles e.g. Ferritin Nanoparticles

The influenza HA antigen described herein may be presented on the surface of nanoparticles, in a strategy known as nano particularization. In one embodiment, the influenza HA ectodomain antigen is presented on the surface of self-assembling protein nanoparticles, suitably ferritin nanoparticles, such as more suitably insect or bacterial ferritin nanoparticles, such as most suitably H. pylori ferritin nanoparticles (such as those disclosed in Corbett et al 2019, WO2013/044203, WO2015/183969 and WO2018/045308). It will be evident that alternative protein nanoparticles known in the art may also be used such as but not limited to lumazine and encapsulin, or other protein nanoparticles including artificially constructed protein nanoparticles.

In a particular embodiment, the influenza HA ectodomain is fused to a heterologous polypeptide, such as ferritin. When ferritin is expressed in a fusion with an HA antigen, it can act as a trimerisation domain for the formation and/or stabilisation of HA trimers. Suitably, the heterologous polypeptide (such as ferritin) and the influenza HA ectodomain monomer are connected by a linker.

Particularization technologies are well known and fusion strategies such as ferritin fusion described here are just one example. Other suitable examples include conjugation, which can be achieved chemically or by using other approaches to protein ligation such as the Streptococcus pyogenes derived system known as SpyTag/SpyCatcher. Multi-component nano particularization technologies can also be applied in which more than one, such as two or more different influenza antigens are displayed. Examples include a fusion with a heterologous polypeptide such as insect ferritin, or combining different antigens in a nanoparticle by means of chemical conjugation. Insect ferritin can be engineered to display two different trimeric antigens in a defined ratio and geometric pattern.

In a further embodiment, the influenza HA ectodomain is in the form of rosette structures such as those described in WO2017/149054. In a particular embodiment, the influenza HA ectodomain is fused to a hydrophobic signal such as a transmembrane domain (which may be a homologous or a heterologous transmembrane domain) and a heterologous trimerization domain, for the purpose of forming rosette structures in vivo or in vitro.

### Immunogenic Fragments

The influenza HA ectodomain antigens described herein also encompass immunogenic fragments of the HA ectodomain regions which fragments comprise the mutations described herein. In one embodiment the influenza HA ectodomain is a polypeptide consisting of an influenza HA ectodomain antigen described herein, or an immunogenic fragment thereof containing a head and a stem domain, and one or more of the mutations (e.g. amino acid substitutions) described herein.

The immunogenic fragment of an influenza HA ectodomain of use in the present invention comprises, such as consists of, a fragment of an influenza HA ectodomain which is capable of eliciting neutralising antibodies and/or a T cell response (such as a CD4 or CD8 T cell response) to influenza virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

Suitably the immunogenic fragment of an influenza HA ectodomain comprises one or more epitopes from a full length influenza HA stem or ectodomain, such as one, two or three or more epitopes.

### HA Epitopes

Certain epitopes on influenza HA are known to be neutralising epitopes for influenza virus. These include the CR9114 stem epitope, the Fl6 (such as Fl6v3) stem epitope, 5A7 stem epitope, the CR8033 epitope and the CR071 epitope (CR9114, CR8033 and CR071 are described for example in Dreyfus et al Science, 2012, 337(6100): 1343-8; Fl6 is described for example in Corti et al Science, 2011, 333(6044): 850-6; and 5A7 in Yagusi et al PLOS Pathogens, 2013, 9(2): e1003150).

In one embodiment, the CR9114 epitope is present in the influenza HA antigen described herein. In another embodiment, the Fl6 (such as Fl6v3) epitope is present in the influenza HA antigen described herein. In another embodiment, both the CR9114 and Fl6 (such as Fl6v3) epitopes are present in the HA antigen. When epitopes are present in the recombinant HA antigen, this means that mAbs directed against those epitopes are capable of binding to the recombinant antigen in a suitable assay.

### Recombinant HA Antigen

A recombinant HA antigen comprises or is encoded by one or more nucleic acids that are derived from a nucleic acid which was artificially constructed. For example, the nucleic acid can comprise, or be encoded by, a cloned nucleic acid formed by joining heterologous nucleic acids.

The recombinant HA antigen includes haemagglutinin-derived sequences HA1 and HA2 of the ectodomain, and may include other non-haemagglutinin derived sequences, for example one or more linker sequences which may each be a flexible linker sequence, or a cleavage site such as a furin cleavage site. A linker sequence may be present for example between the HA1 and HA2 regions of the recombinant HA. A linker sequence can facilitate the independent folding of the HA domains. The linker sequence may be an amino acid sequence that is synthesized as part of a recombinant fusion protein. Linkers, especially short linkers, may be present between the ectodomain and the trimerization domain and/or between the trimerization domain and the His tag, if present. In other embodiments a chemical linker is used to connect synthetically or recombinantly produced sub-sequences. Such flexible linkers are known to those skilled in the art. A cleavage site, such as a furin cleavage site, may be present between the HA1 and HA2 regions of the HA. A furin cleavage site can be used to allow activation e.g. from a vector technology. This may improve antigen representativity and immunogenicity.

The recombinant HA antigen may further comprise, in addition to or as an alternative to linkers, polypeptide sub-sequences from proteins which are unrelated to haemagglutinin, for example a sequence with affinity to a known antibody to facilitate affinity purification and/or detection. Such detection and purification-facilitating domains include, but are not limited to, metal chelating peptides such as polyhistidine tracts and histidine-tryptophan modules that allow purification on immobilized metals and protein A domains that allow purification on immobilized immunoglobulin. Examples include heterologous fusion sequences encoding gD tags, AviTag, c-Myc epitopes, polyhistidine tags, fluorescent proteins (e.g. GFP), beta-galactosidase protein or glutathione S transferase or any other sequence useful for detection or purification of the fusion protein expressed in or on a cell. A preferred further polypeptide sequence is a polyhistidine tag, such as a four or a six or an eight or a ten-histidine tag, in particular a six-histidine tag. The inclusion of a cleavable linker sequence between the purification domain (e.g. polyhistidine tag) and the HA antigen may be useful to facilitate purification. For example, an enzyme cleavage site, such as a TEV cleavage site or a thrombin cleavage site, may be included between the further polypeptide and the rest of the recombinant HA sequence.

A cleavable linker sequence, for example an enzyme cleavage site such as a TEV cleavage site or a thrombin cleavage site may alternatively or additionally be included between the trimerization domain and the rest of the recombinant HA sequence. This can allow the trimerization domain to be removed in the final recombinant HA. Hence, the recombinant HA described herein may comprise or consist of (in order) an ectodomain of HA, heterologous trimerization domain, purification tag (e.g. polyhistidine tag) and optionally a cleavable linker sequence i) between the purification tag and the rest of the recombinant HA and/or ii) between the trimerization domain and the HA ectodomain.

In some embodiments, the His tag and optionally the trimerization domain (e.g. foldon or GCN4) if present, are cleaved following expression of the protein and thus are not present in the final HA antigen.

For example, the recombinant HA antigen described herein may comprise or consist of i) an amino acid sequence comprising the ectodomain of HA, and ii) the heterologous trimerization domain (foldon) in SEQ ID NO: 9 or a derivative thereof that maintains the ability to induce recombinant HA monomers to form trimers. In particular, the recombinant HA ectodomain antigen may comprise, such as consist of, a sequence having at least 80% identity, such as 90% or greater, such as 95% or greater, such as 98% or greater, such as 99% or greater sequence identity to any one of the amino acid sequences of SEQ ID NOS: 3, 4, 5, 6, 7, or 8, more suitably SEQ ID NO: 3 or 6. The amino acid sequences of SEQ ID NOS: 3-8 all contain the foldon sequence and poly His tag, either or both of which are optionally removed to provide the final HA antigen. Alternatively, the recombinant HA ectodomain antigen may comprise, such as consist of, a sequence having at least 80% identity, such as 90% or greater, such as 95% or greater, such as 98% or greater, such as 99% or greater sequence identity to any one of the amino acid sequences of SEQ ID NOS: 17 to 42, more suitably SEQ ID NO: 19, 21, 26, 34 or 42. The amino acid sequence of SEQ ID NOS: 17-42 all contain foldon sequence and poly His tag, either or both of which are optionally removed to provide the final HA antigen.

For example, the recombinant HA antigen described herein may comprise i) an amino acid sequence comprising the HA antigen, and ii) a heterologous trimerization domain e.g. foldon as shown in SEQ ID NO: 9 or a derivative thereof that maintains the ability to induce recombinant HA monomers to form trimers. In particular, the recombinant HA ectodomain antigen may comprise or consist of any one of the amino acid sequences shown in SEQ ID NOS: 3 to 8 and 17 to 42. These sequences all contain a foldon, which is optionally removed to provide the final HA antigen.

A recombinant HA antigen described herein presented in a nanoparticle may comprise for example i) an amino acid sequence comprising the HA ectodomain antigen, such as any of the amino acid sequences shown in SEQ ID NOS: 3 to 8 and 17 to 42, minus the foldon; and optionally ii) a heterologous polypeptide capable of forming a nanoparticle such as ferritin. In one embodiment the recombinant HA antigen is an ectodomain antigen described herein, fused to H. pylori ferritin.

A recombinant HA antigen described herein delivered as nucleic acid such as mRNA, may comprise i) an amino acid sequence comprising HA ectodomain antigen, and optionally ii) a transmembrane domain (homologous i.e. derived from the same influenza strain or heterologous i.e. derived from another influenza strain or another source, and optionally trimeric) such that the antigen is membrane anchored once the antigen is expressed, or a heterologous polypeptide capable of forming a nanoparticle, such as ferritin, such that the antigen once expressed is presented on the surface of nanoparticles.

### Polynucleotide Encoding HA Antigen

A polynucleotide construct encoding the recombinant HA antigen may include a signal sequence. Typically, the signal sequence is appropriate for the host cell in which the recombinant HA is expressed. In one embodiment, the wild type signal peptide sequence is used. In another embodiment, a heterologous signal peptide sequence is used.

Accordingly, a polynucleotide encoding the recombinant influenza HA antigen described herein may comprise a sequence encoding HA ectodomain, a heterologous trimerization domain (e.g. foldon), a purification tag (e.g. polyhistidine tag) and a signal peptide (e.g. SEQ ID NO: 10 or 43), such as in the order: a signal peptide (e.g. SEQ ID NO: 10 or 43), HA ectodomain, heterologous trimerization domain (e.g. foldon), purification tag (e.g. polyhistidine tag). In another example, the polynucleotide encoding the recombinant HA antigen described herein comprises a sequence encoding (in order): a signal peptide (e.g. SEQ ID NO: 10 or 43), the HA ectodomain, a cleavable linker sequence (e.g. TEV cleavage site), a heterologous trimerization domain (e.g. foldon), and a purification tag (e.g. polyhistidine tag).

The polynucleotide sequence encoding the recombinant influenza HA antigen may comprise i) a polynucleotide sequence encoding the HA ectodomain and ii) SEQ ID NO: 9 encoding the foldon or a derivative of the foldon that maintains the ability to induce expressed recombinant HA monomers to form trimers.

In particular embodiments, the polynucleotide sequence encoding the recombinant HA antigen described herein comprises or consists of a sequence encoding the polypeptide of any one of SEQ ID NOs: 3 to 8 or 17 to 42, such as for example a polynucleotide sequence of SEQ ID NOs: 11 to 16 or 44 to 69.

In another embodiment, the polynucleotide sequence encoding the recombinant influenza HA antigen comprises a sequence encoding the ectodomain, and a transmembrane domain. In one embodiment the transmembrane domain is the native influenza HA transmembrane or a functional derivative that anchors the antigen in a membrane. In one embodiment, the polynucleotide sequence is formulated for delivery as a nucleic acid vaccine.

In another embodiment, the polynucleotide sequence encoding the recombinant influenza HA antigen comprises a sequence encoding the ectodomain, and a sequence encoding a polypeptide capable of forming a nanoparticle. In one embodiment the polypeptide capable of forming a nanoparticle is ferritin. In one embodiment, the polynucleotide sequence is formulated for delivery as a nucleic acid vaccine.

Nucleic acid-based vaccines are contemplated herein for any of the influenza HA antigens described. The nucleic acid may, for example, be RNA (i.e. an RNA-based vaccine or mRNA delivery platform) or DNA (i.e. a DNA-based vaccine, such as a plasmid DNA vaccine), including viral vectors. The sequence of the nucleic acid molecule may be modified, e.g. to increase the efficacy of expression or replication of the nucleic acid, or to provide additional stability or resistance to degradation, or to reduce reactogenicity or to activate the interferon pathway impacting antigen expression.

Messenger RNA (mRNA) can direct the cellular machinery of a subject to produce proteins. The term mRNA as used herein includes conventional mRNA or mRNA analogues, such as those containing modified backbones or modified bases (e.g. pseudouridine, or the like). mRNA, may or may not have a 5 cap. The mRNA may encode more than one antigen. For example, the mRNA encoding an HA antigen as described herein may encode only the HA antigen or it may encode a second HA antigen or additional proteins. Where additional proteins are encoded, mRNA may be polycistronic.

mRNA may be non-replicating or may be replicating, also known as self-amplifying. A self-amplifying mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest.

mRNA may also be codon optimised. In some embodiments, mRNA may be codon optimised for expression in human cells.

A range of carrier systems have been described which encapsulate or complex mRNA in order to facilitate mRNA delivery and consequent expression of encoded antigens as compared to mRNA which is not encapsulated or complexed. The present invention may utilise any suitable carrier system. Particular carrier systems include lipid nanoparticles (LNPs) which are non-virion liposome particles in which mRNA can be encapsulated; cationic nanoemulsion (CNE) delivery systems in which cationic oil-in-water emulsions can be used to deliver the mRNA to the interior of a cell; and lipidoid-coated iron oxide nanoparticles (LION) which are capable of delivering mRNA into cells and may be aided after administration to a subject by application of an external magnetic field. In one embodiment the mRNA encoding an HA antigen as described herein is encapsulated or complexed in a carrier system selected from LNPs, CNE and LION.

### Trimerisation Domains

A suitable trimerization domain is one that induces the recombinant HA antigen monomers to form trimers and increases stability. Suitably the trimerization domain is or is derived from the natural trimerization domain of the T4 phage fibritin “foldon”. A foldon sequence may be used which forms a β-propeller structure comprising the C terminus of the fibritin domain of the T4 bacteriophage. For example, the trimerization domain may comprise or consist of the foldon amino acid sequence shown in SEQ ID NO: 9 or a derivative of this sequence that maintains the ability to induce recombinant monomers to form trimers.

Another suitable trimerisation domain is a leucine zipper trimerization motif derived from the yeast transcription activator GCN4. Further suitable trimerization domains include chloramphenicol acetyl transferase (CAT). The trimerization domain is placed at the C terminus of the HA ectodomain, i.e. at the stem end of the HA. Further trimerization domains include a human-derived trimerization domain such as Trimer-Tag, or an HIV-derived trimerization domain. Typically, the trimerization domain is fused via a short linker region to the HA sequence. The region between the trimerization domain and the HA sequence may include a cleavable linker sequence, so it is possible to isolate the HA sequence from the trimerization domain at a later stage. Thus, the HA sequence may be linked (e.g. in order), optionally via a linker sequence, to a heterologous sequence comprising a protease cleavage site, the trimerization domain and a purification tag such as a histidine tag to aid in purification. Such heterologous trimerization domains may be linked to HA sequences by techniques known in the art, such as molecular cloning.

### Preparation of Recombinant HA Antigen

Use of recombinant DNA technology to produce influenza vaccines offers several advantages. These include potentially avoiding the steps of adaptation and passage of infectious viruses in eggs, and production of more highly purified protein under safer and more stringently controlled conditions. Moreover, no virus inactivation step has to be included. Any suitable cloning and expression system may be used to recombinantly produce the recombinant HA antigen.

Nucleotide sequences encoding the recombinant HA antigens of the invention may be synthesized, and/or cloned and expressed according to techniques well known to those in the art. See for example, Sambrook, et al. Molecular Cloning, A Laboratory Manual, Vols. 1-3, Cold Spring Harbor Press, Cold Spring Harbor, NY (1989). In some embodiments, the polynucleotide sequences will be codon optimised for a particular recipient host cell using standard methodologies. For example, a DNA construct encoding a haemagglutinin sequence can be codon optimised for expression in other hosts e.g. bacteria, mammalian or insect cells. Suitable host cells may include bacterial cells such as E. Coli, fungal cells such as yeast, insect cells such as Drosophila S2, Spodoptera Sf9, Sf00+ or Hi-5 and animal cells such as CHO.

Suitably, the HA antigens described herein are expressed in a eukaryotic cell, such as a mammalian cell, e.g. a human cell such as HEK293T cell, non-human mammalian cell such as CHO cell, or an insect cell, optionally further comprising purifying/isolating the recombinant HA from the cell.

Haemagglutinin sequences may be produced by standard recombinant methods known in the art, such as polymerase chain reaction (PCR) or reverse transcriptase PCR, reverse engineering, or the DNA can be synthesized. For PCR, primers can be prepared using haemagglutinin nucleotide sequences that are available in public databases.

### Sequence Identity

Identity with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA or MUSCLE or CLUSTALO, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 (a standard scoring matrix; see Dayhoff et al (1978) A model of evolutionary change in proteins, in Atlas of Protein Sequence and Structure, vol. 5, supp. 3) can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences (e.g., divided by the length of the alignment).

### Influenza Strains

A ‘type’ of influenza virus refers to influenza type A, influenza type B or influenza type C. The designation of a virus as a specific type relates to sequence difference in the respective MI (matrix) protein or NP (nucleoprotein). Type A influenza viruses are further divided into group 1 and group 2. These groups, also referred to as clades, are further divided into subtypes, which refers to classification of a virus based on the sequence of its HA protein. Examples of current commonly recognized subtypes are H1, H2, H3, H4, H5, H6, H7, H8, H9, H10, H11, H12, H13, H14, H15, H16, H17 and H18. Of these, the group 1 influenza A subtypes are H1, H2, H5, H6, H8, H9, H11, H12, H13, H16, H17 and H18. Group 2 influenza A subtypes are H3, H4, H7, H10, H14 and H15. Finally, the term ‘strain’ refers to viruses within a subtype that differ from one another in that they have small, genetic variations in their genome.

The HA ectodomain sequence of the recombinant HA antigens described herein may be from any subtype of influenza A e.g. H1, H2, H3, H4, H5, H6, H7, H8, H9, H10, H11, H12, H13, H14, H15, H16, H17, H18. In one embodiment, the HA sequence of the HA antigen is from a strain selected from the group 1 influenza A subtypes, which include H1, H2, H5, H6, H8, H9, H11, H12, H13, H16, H17 and H18. In a particular embodiment, the HA sequence is from a H1 strain from the human population, such as Bri18. In a further embodiment, the HA sequence of the HA antigen is from a strain selected from the group 2 influenza subtypes, which include H3, H4, H7, H10, H14 and H15.

In one embodiment, the HA sequence is from naturally occurring HA from a non-pandemic strain. In one embodiment, the HA sequence is from naturally occurring HA from a circulating influenza virus or a strain recommended by the WHO for seasonal flu vaccines. For example, the circulating or vaccine-recommended influenza virus may be a strain identified by WHO as a circulating or vaccine-recommended seasonal influenza virus strain, or identified by the WHO in a previous season as a circulating or vaccine-recommended seasonal strain. In one embodiment, the HA sequence is from a strain identified by the WHO as having the potential to cause an epidemic in a subsequent influenza season. In one embodiment, the HA sequence is from a strain which is a new influenza virus strain against which the large majority of the human population has no immunity. In one embodiment, the HA ectodomain sequence is from a strain which has the potential to cause a pandemic. Typically, the WHO identifies and publicises such strains.

### Additional Antigens

The present invention may involve a plurality of antigenic components, for example with the objective to elicit a broad immune response to influenza virus. Consequently, more than one antigen may be present, more than one polynucleotide encoding an antigen may be present, one polynucleotide encoding more than one antigen may be present or a mixture of antigen(s) and polynucleotide(s) encoding antigen(s) may be present. Polysaccharides such as polysaccharide conjugates may also be present.

By the term antigen is meant a polypeptide which is capable of eliciting an immune response. Suitably the antigen comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

### Vaccine Delivery

It will be evident that the recombinant HA antigen described herein can be delivered in any suitable vaccine delivery mode, such as in the form of a protein, or in the form of nucleic acid, including nucleic acid delivery platforms such as DNA (including for example a viral delivery platform such as adenovirus) or RNA including for example mRNA, optionally formulated in a carrier system. Suitable delivery systems include viral vectors such as adenovirus vectors. In any case, the delivery mode will include a pharmaceutically acceptable diluent or carrier. Optionally one or more adjuvants may be used.

### Immunogenic Composition

In one aspect, an immunogenic composition comprising an influenza HA antigen or polynucleotide as described herein and a pharmaceutically acceptable carrier is provided.

In one embodiment, the immunogenic composition comprising a HA antigen as described herein further comprises an adjuvant. Preferably, the adjuvant is an oil-in-water emulsion adjuvant. Oil in water emulsion adjuvants are well known in the art and are described below in more detail.

In one embodiment, the immunogenic composition comprising a HA polynucleotide encoding a HA antigen as described herein further comprises a polynucleotide carrier or delivery system.

In one embodiment, the immunogenic composition is monovalent, i.e. comprises influenza HA antigen or polynucleotide encoding it, from only one influenza A strain. In alternative embodiments, the composition is multivalent, i.e. comprises influenza virus antigens from multiple strains. For example, the composition may be bivalent, trivalent or quadrivalent, e.g. may contain two or three seasonal strains together with the recombinant HA antigen described here. For example, the composition may contain the antigen or polynucleotide described here, together with one further A strain antigen or polynucleotide and optionally one or two B strain antigens or polynucleotides.

In one embodiment, the immunogenic composition is an improved seasonal influenza vaccine in which the HA antigen is capable of inducing an immune response against at least one other influenza strain, from the same or a different subtype e.g. influenza A strain haemagglutinin subtype. In further embodiments, the immunogenic composition is capable of inducing an immune response against two or three or four or more different strains including one or more from each of two different subtypes such as H1 and H3. In one embodiment, the immunogenic composition comprises an HA antigen from H1 which is capable of inducing an immune response against one or more other H1 strains. In one embodiment, the immunogenic composition comprises an HA antigen from H1 which is capable of inducing a heterosubtypic immune response against one or more group 1 subtypes such as H2, H5 or H9. In one embodiment, the immunogenic composition comprises an HA antigen from H1 which is capable of inducing a heterosubtypic immune response against one or more group 2 subtypes such as H10.

In one embodiment there is provided an immunogenic composition comprising (i) an influenza HA ectodomain antigen in trimeric form, said antigen comprising one or more stabilising mutations described herein in the coiled coil region; and (ii) a squalene-based adjuvant.

### Adjuvant

In one embodiment, an immunogenic composition of the invention comprises an adjuvant. In particular, the adjuvant may be an emulsion, such as an oil-in-water emulsion. Optionally, other immunostimulants may be present in the oil-in-water emulsion. In a specific embodiment, an oil-in-water emulsion comprises a metabolisable, non-toxic oil such as squalene or squalane, optionally a tocol such as tocopherol in particular alpha tocopherol, and an emulsifier (or surfactant) such as the non-ionic surfactant polyoxyethylene sorbitan monooleate (TWEEN-80™ or polysorbate 80™) Mixtures of surfactants can be used such as polyoxyethylene sorbitan monooleate/sorbitan trioleate (SPAN 85™) mixtures, or polyoxyethylene sorbitan monooleate/t-octylphenoxypolyethoxyethanol (TRITON X-100™) mixtures.

In one aspect, the oil-in-water emulsion has one of the following compositions:
From 0.5 to 11 mg squalene, from 0.05 to 5% polyoxythylene sorbitan monooleate (TWEEN-80™ or POLYSORBATE 80™) and optionally, from 2 to 12% alpha-tocopherol; or
About 5% squalene, about 0.5% polyoxyethylene sorbitan monooleate (TWEEN-80™ or POLYSORBATE 80™) and about 0.5% sorbitan trioleate (SPAN 85™). This adjuvant is called MF59.

Squalene emulsion adjuvants are described in more detail below.

An alternative adjuvant that may be used comprises an immunologically active saponin fraction derived from the bark of Quillaja Saponaria Molina (e.g. QS21) presented in the form of a liposome and a lipopolysaccharide (e.g. 3D-MPL), optionally further including a sterol (cholesterol). In one embodiment, the adjuvant comprises or consists of a saponin (e.g. QS21) presented in the form of a liposome, a lipid A derivative such as 3D-MPL and a sterol (e.g. cholesterol). The liposomes suitably contain a neutral lipid, for example, phosphatidylcholine, dioleoyl phosphatidylcholine (DOPC) or dilauryl phosphatidylcholine. The liposomes may also contain a charged lipid which increases the stability of the liposome-QS21 structure for liposomes composed of saturated lipids. An example of such an adjuvant is AS01, which comprises 3D-MPL and QS21 in a quenched form with cholesterol, and can be made as described in WO96/33739. Either the AS01B or AS01E forms of this adjuvant may be used. The AS01 B adjuvant comprises liposomes, which in turn comprise dioleoyl phosphatidylcholine (DOPC), cholesterol and 3D-MPL (in an amount of approximately 1000 micrograms DOPC, 250 micrograms cholesterol and 50 micrograms 3D-MPL per vaccine dose), QS21 (50 micrograms/dose), phosphate NaCl buffer and water to a volume of 0.5 ml.

The AS01E adjuvant comprises the same ingredients than AS01B but at a lower concentration in an amount of approximately 500 micrograms DOPC, 125 micrograms cholesterol, 25 micrograms 3D-MPL and 25 micrograms QS21, phosphate NaCl buffer and water to a volume of 0.5 ml.

In one embodiment, the influenza HA ectodomain antigen is in physical association with an adjuvant e.g. liposomes of AS01 or emulsion of a squalene-containing adjuvant such as AS03.

### Squalene Emulsion Adjuvant

The term ‘squalene emulsion adjuvant’ as used herein refers to a squalene containing oil-in-water emulsion adjuvant.

Squalene, is a branched, unsaturated terpenoid ([(CH3)2C[=CHCH2CH2C(CH3)]2=CHCH2-]2; C30H50; 2,6,10,15,19,23-hexamethyl-2,6,10,14,18,22-tetracosahexaene; CAS Registry Number 7683-64-9). Squalene is readily available from commercial sources or may be obtained by methods known in the art. Squalene shows good biocompatibility and is readily metabolised.

The squalene emulsion adjuvant may comprise one or more tocopherols, suitably wherein the weight ratio of squalene to tocopherol is 20 or less (i.e. 20 weight units of squalene or less per weight unit of tocopherol or, alternatively phrased, at least 1 weight unit of tocopherol per 20 weight units of squalene).

Any of the α, β, γ, δ, ε and/or ξ tocopherols can be used, but α-tocopherol (also referred to herein as alpha-tocopherol) is typically used. D-alpha-tocopherol and D/L-alpha-tocopherol can both be used. Tocopherols are readily available from commercial sources or may be obtained by methods known in the art. In some embodiments the squalene emulsion adjuvant contains alpha-tocopherol, especially D/L-alpha-tocopherol.

Squalene emulsion adjuvants will typically have a submicron droplet size. Droplet sizes below 200 nm are beneficial in that they can facilitate sterilisation by filtration. There is evidence that droplet sizes in the 80 to 200 nm range are of particular interest for potency, manufacturing consistency and stability reasons (Klucker, 2012; Shah, 2014; Shah, 2015; Shah, 2019). Suitably the squalene emulsion adjuvant has an average droplet size of less than 1 um, especially less than 500 nm and in particular less than 200 nm. Suitably the squalene emulsion adjuvant has an average droplet size of at least 50 nm, especially at least 80 nm, in particular at least 100 nm, such as at least 120 nm. The squalene emulsion adjuvant may have an average droplet size of 50 to 200 nm, such as 80 to 200 nm, especially 120 to 180 nm, in particular 140 to 180 nm, such as about 160 nm.

Uniformity of droplet sizes is desirable. A polydispersity index (Pdl) of greater than 0.7 indicates that the sample has a very broad size distribution and a reported value of 0 means that size variation is absent, although values smaller than 0.05 are rarely seen. Suitably the squalene emulsion adjuvant has a polydispersity of 0.5 or less, especially 0.3 or less, such as 0.2 or less.

The droplet size, as used herein, means the average diameter of oil droplets in an emulsion and can be determined in various ways e.g. using the techniques of dynamic light scattering and/or single-particle optical sensing, using an apparatus such as the Accusizer™ and Nicompi™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions Schartl, 2007. Dynamic light scattering (DLS) is the preferred method by which droplet size is determined. The preferred method for defining the average droplet diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to average droplet size should be taken as an intensity-weighted average, and ideally the Z-average. Pdl values are easily provided by the same instrumentation which measures average diameter.

In order to maintain a stable submicron emulsion, one or more emulsifying agents (i.e. surfactants) are generally required. Surfactants can be classified by their ‘HLB’ (Griffin's hydrophile/lipophile balance), where a HLB in the range 1-10 generally means that the surfactant is more soluble in oil than in water, whereas a HLB in the range 10-20 means that the surfactant is more soluble in water than in oil. HLB values are readily available for many surfactants of interest or can be determined experimentally, e.g. polysorbate 80 has a HLB of 15.0 and TPGS has a HLB of 13 to 13.2. Sorbitan trioleate has a HLB of 1.8. When two or more surfactants are blended, the resulting HLB of the blend is typically calculated by the weighted average e.g. a 70/30 wt % mixture of polysorbate 80 and TPGS has a HLB of (15.0×0.70)+(13×0.30) i.e. 14.4. A 70/30 wt % mixture of polysorbate 80 and sorbitan trioleate has a HLB of (15.0×0.70)+(1.8×0.30) i.e. 11.04.

Surfactant(s) will typically be metabolisable (biodegradable) and biocompatible, being suitable for use as a pharmaceutical. The surfactant can include ionic (cationic, anionic or zwitterionic) and/or non-ionic surfactants. The use of only non-ionic surfactants is often desirable, for example due to their pH independence. The invention can thus use surfactants including, but not limited to:
the polyoxyethylene sorbitan ester surfactants (commonly referred to as the Tweens or polysorbates), such as polysorbate 20 and polysorbate 80, especially polysorbate 80;
copolymers of ethylene oxide (EO), propylene oxide (PO), and/or butylene oxide (BO), sold under the DOWFAX™, Pluronic™ (e.g. F68, F127 or L121 grades) or Synperonic™ tradenames, such as linear EO/PO block copolymers, for example poloxamer 407, poloxamer 401 and poloxamer 188;
octoxynols, which can vary in the number of repeating ethoxy (oxy-1,2-ethanediyl) groups, with octoxynol-9 (Triton X-100, or t-octylphenoxypolyethoxyethanol) being of particular interest;
(octylphenoxy)polyethoxyethanol (IGEPAL CA-630/NP-40);
phospholipids such as phosphatidylcholine (lecithin);
polyoxyethylene fatty ethers derived from lauryl, cetyl, stearyl and oleyl alcohols (known as Brij surfactants), such as polyoxyethylene 4 lauryl ether (Brij 30, Emulgen 104P), polyoxyethylene-9-lauryl ether and polyoxyethylene 12 cetyl/stearyl ether (Eumulgin™ B1, cetereth-12 or polyoxyethylene cetostearyl ether);
sorbitan esters (commonly known as the Spans), such as sorbitan trioleate (Span 85), sorbitan monooleate (Span 80) and sorbitan monolaurate (Span 20);
or tocopherol derivative surfactants, such as alpha-tocopherol-polyethylene glycol succinate (TPGS).

Many examples of pharmaceutically acceptable surfactants are known in the art e.g. see Handbook of Pharmaceutical Excipients 6th edition, 2009. Methods for selecting and optimising the choice of surfactant used in a squalene emulsion adjuvant are illustrated in Klucker, 2012. In general, the surfactant component has a HLB between 10 and 18, such as between 12 and 17, in particular 13 to 16. This can be typically achieved using a single surfactant or, in some embodiments, using a mixture of surfactants. Surfactants of particular interest include: poloxamer 401, poloxamer 188, polysorbate 80, sorbitan trioleate, sorbitan monooleate and polyoxyethylene 12 cetyl/stearyl ether either alone, in combination with each other or in combination with other surfactants. Especially of interest are polysorbate 80, sorbitan trioleate, sorbitan monooleate and polyoxyethylene 12 cetyl/stearyl ether either alone, or in combination with each other. A particular surfactant of interest is polysorbate 80. A particular combination of surfactants of interest is polysorbate 80 and sorbitan trioleate. A further combination of surfactants of interest is sorbitan monooleate and polyoxyethylene cetostearyl ether.

In certain embodiments the squalene emulsion adjuvant comprises one surfactant, such as polysorbate 80. In some embodiments the squalene emulsion adjuvant comprises two surfactants, such as polysorbate 80 and sorbitan trioleate or sorbitan monooleate and polyoxyethylene cetostearyl ether. In other embodiments the squalene emulsion adjuvant comprises three or more surfactants, such as three surfactants.

If tocopherol is present, the weight ratio of squalene to tocopherol may be 20 or less, such as 10 or less. Suitably the weight ratio of squalene to tocopherol is 0.1 or more. Typically the weight ratio of squalene to tocopherol is 0.1 to 10, especially 0.2 to 5, in particular 0.3 to 3, such as 0.4 to 2.

Suitably, the weight ratio of squalene to tocopherol is 0.72 to 1.136, especially 0.8 to 1, in particular 0.85 to 0.95, such as 0.9.

If surfactant is present, typically the weight ratio of squalene to surfactant is 0.73 to 6.6, especially 1 to 5, in particular 1.2 to 4. Suitably, the weight ratio of squalene to surfactant is 1.71 to 2.8, especially 2 to 2.4, in particular 2.1 to 2.3, such as 2.2.

The amount of squalene in a single dose, such as a human dose, of squalene emulsion adjuvant is typically at least 1.2 mg. Generally, the amount of squalene in a single dose, such as a human dose, of squalene emulsion adjuvant is 50 mg or less. The amount of squalene in a single dose, such as a human dose, of squalene emulsion adjuvant may be 1.2 to 20 mg, in particular 1.2 to 15 mg. The amount of squalene in a single dose, such as a human dose, of squalene emulsion adjuvant may be 1.2 to 2 mg, 2 to 4 mg, 4 to 8 mg or 8 to 12.1 mg. For example, the amount of squalene in a single dose, such as a human dose, of squalene emulsion adjuvant may be 1.21 to 1.52 mg, 2.43 to 3.03 mg, 4.87 to 6.05 mg or 9.75 to 12.1 mg.

If tocopherol is present, the amount of tocopherol in a single dose, such as a human dose, of squalene emulsion adjuvant is typically at least 1.3 mg. Generally, the amount of tocopherol in a single dose, such as a human dose, of squalene emulsion adjuvant is 55 mg or less. The amount of tocopherol in a single dose, such as a human dose, of squalene emulsion adjuvant may be 1.3 to 22 mg, in particular 1.3 to 16.6 mg. The amount of tocopherol in a single dose, such as a human dose, of squalene emulsion adjuvant may be 1.3 to 2 mg, 2 to 4 mg, 4 to 8 mg or 8 to 13.6 mg. For example, the amount of tocopherol in a single dose, such as a human dose, of squalene emulsion adjuvant may be 1.33 to 1.69 mg, 2.66 to 3.39 mg, 5.32 to 6.77 mg or 10.65 to 13.53 mg.

If surfactant is present, the amount of surfactant in a single dose, such as a human dose, of squalene emulsion adjuvant is typically at least 0.4 mg. Generally, the amount of surfactant in a single dose, such as a human dose, of squalene emulsion adjuvant is 18 mg or less. The amount of surfactant in a single dose, such as a human dose, of squalene emulsion adjuvant may be 0.4 to 9.5 mg, in particular 0.4 to 7 mg. The amount of surfactant in a single dose, such as a human dose, of squalene emulsion adjuvant may be 0.4 to 1 mg, 1 to 2 mg, 2 to 4 mg or 4 to 7 mg. For example, the amount of surfactant in a single dose, such as a human dose, of squalene emulsion adjuvant may be 0.54 to 0.71 mg, 1.08 to 1.42 mg, 2.16 to 2.84 mg or 4.32 to 5.68 mg.

In certain embodiments the squalene emulsion adjuvant may consist essentially of squalene, surfactant and water. In certain other embodiments the squalene emulsion adjuvant may consist essentially of squalene, tocopherol, surfactant and water. Squalene emulsion adjuvants may contain additional components as desired or required depending upon the intended final presentation and vaccination strategy, such as buffers and/or tonicity modifying agents, for example modified phosphate buffered saline (disodium phosphate, potassium biphosphate, sodium chloride and potassium chloride).

High pressure homogenization (HPH or microfluidisation) may be applied to yield squalene emulsion adjuvants comprising tocopherol which demonstrate uniformly small droplet sizes and long-term stability (see EP0868918 and WO2006/100109). Briefly, oil phase composed of squalene and tocopherol may be formulated under a nitrogen atmosphere. Aqueous phase is prepared separately, typically composed of water for injection or phosphate buffered saline, and polysorbate 80. Oil and aqueous phases are combined, such as at a ratio of 1:9 (volume of oil phase to volume of aqueous phase) before homogenisation and microfluidisation, such as by a single pass through an in-line homogeniser and three passes through a microfluidiser (at around 15000 psi). The resulting emulsion may then be sterile filtered, for example through two trains of two 0.5/0.2 um filters in series (i.e. 0.5/0.2/0.5/0.2), see WO2011/154444. Operation is desirably undertaken under an inert atmosphere, e.g. nitrogen. Positive pressure may be applied, see WO2011/154443.

International patent application WO2020160080 and Lodaya R, et al.: J Control Release (2019) 316:12-21 describe squalene emulsion adjuvants comprising tocopherol which are self-emulsifying adjuvant systems (SEAS) and their manufacture.

### Vaccination Regimes, Dosing and Efficacy Criteria

Suitably, the immunogenic compositions described herein are a standard 0.5 ml injectable dose in most cases, and contain 15 μg or less, of hemagglutinin antigen component from an influenza virus strain, as measured by single radial immunodiffusion (SRD) (J. M. Wood et al.: J. Biol. Stand. 5 (1977) 237-247; J. M. Wood et al., J. Biol. Stand. 9 (1981) 317-330). Suitably the vaccine dose volume will be from 0.25 ml to 1 ml, in particular a standard 0.5 ml, or 0.7 ml vaccine dose volume. Slight adaptation of the dose volume will be made routinely depending on the HA concentration in the original bulk sample and depending also on the delivery route with smaller doses being given by the intranasal or intradermal route. Immunogenic compositions for use according to the invention may contain a low amount of HA antigen—e.g. any of 1, 2, 3, 4, 5, 6, 7, 8, 9, 10, 11, 12, 13, 14 μg of HA per influenza virus strain or which does not exceed 15 μg of HA per strain. Said low amount of HA amount may be as low as practically feasible provided that it allows formulation of a vaccine which meets the international e.g. EU or FDA criteria for efficacy. A suitable low amount of HA is from 1 to 7.5 μg of HA per influenza virus strain, suitably from 3.5 to 5 μg, such as 3.75 or 3.8 μg of HA per influenza virus strain, typically about 5 μg of HA per influenza virus strain. Another suitable amount of HA is from 0.1 to 5 μg of HA per influenza virus strain, suitably from 1.0 to 2 μg of HA per influenza virus strain, such as 1.9 μg of HA per influenza virus strain.

The influenza medicament (e.g. immunogenic composition) described herein suitably meets certain international criteria for vaccines. Standards are applied internationally to measure the efficacy of influenza vaccines.

Serological variables are assessed according to criteria of the European Agency for the Evaluation of Medicinal Products for human use (CHMP/BWP/214/96, Committee for Proprietary Medicinal Products (CPMP)) or as updated.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the recombinant HA antigen may therefore be part of a multi-dose administration regime. For example, the recombinant HA antigen may be provided as a priming dose in a multidose regime, especially a two- or three-dose regime, in particular a two-dose regime. The recombinant HA antigen may be provided as a boosting dose in a multidose regime, especially a two- or three-dose regime, such as a two-dose regime.

Priming and boosting doses may be homologous or heterologous. Consequently, the recombinant HA antigen may be provided as a priming dose and boosting dose(s) in a homologous multidose regime, especially a two- or three-dose regime, in particular a two-dose regime. Alternatively, the recombinant HA antigen may be provided as a priming dose or boosting dose in a heterologous multidose regime, especially a two- or three-dose regime, in particular a two-dose regime, and the boosting dose(s) may be different (e.g. a different HA antigen; or an alternative antigen presentation such as protein or virally vectored antigen—with or without adjuvant).

The time between doses may be two weeks to six months, such as three weeks to three months. Periodic longer-term booster doses may be also be provided, such as every 2 to 10 years.

### Methods of Treatment

In a further embodiment, the immunogenic composition comprising the HA antigen or polynucleotide is for use in medicine, such as for use in the prevention of, or vaccination against, influenza e.g. administered to a person (e.g. subject) at risk of influenza infection.

In a yet further embodiment, the immunogenic composition comprising the antigen or polynucleotide is for use in the prevention of influenza caused by a different haemagglutinin subtype than the subtype on which the HA antigen was based. For example, a HA antigen from H1 could be used for protection against influenza caused by a non-H1 influenza A strain virus e.g. from a group 1 subtype such as H2, H5 or H9, or vice versa.

In a further aspect, there is provided a method of prevention and/or treatment of influenza disease, comprising the administration of a recombinant HA antigen or immunogenic composition as described herein to a person in need thereof, e.g. to a person (e.g. subject) at risk of influenza infection, e.g. an elderly person (age 50 or over, particularly age 65 or over).

In one embodiment of the above-described method or use, less than 15 micrograms, such as from 3.75 to 10 micrograms of HA is administered per dose.

In one aspect, the invention provides the recombinant HA antigen described herein at a dose of below 10 micrograms, or below 8 micrograms, or from 1-7.5 micrograms, or from 1-5 micrograms of recombinant HA for use in a vaccination regimen for the prevention of influenza, wherein the hemagglutinin sequences are from, or derived from a strain of influenza identified by an international organisation such as the WHO that monitors outbreaks of influenza virus, as associated with an outbreak or as having the potential to be associated with a future outbreak.

### Routes of Administration

The composition of the invention may be administered by any suitable delivery route, such as intradermal, mucosal (e.g. intranasal), oral, intramuscular (IM) or subcutaneous. Other delivery routes are well known in the art.

The intramuscular delivery route is particularly suitable for the immunogenic composition, in particular for the adjuvanted immunogenic composition. The composition may be presented in a mono-dose container, or alternatively, a multi-dose container. In this instance an antimicrobial preservative such a thiomersal may be present to prevent contamination during use. A thiomersal concentration of 5 μg/0.5 ml dose (i.e. 10 μg/ml) or 10 μg/0.5 ml dose (i.e. 20 μg/ml) is suitably present. A suitable IM delivery device could be used such as a needle-free liquid jet injection device, for example the Biojector 2000 (Bioject, Portland, OR). Alternatively, a pen-injector device, such as is used for at-home delivery of epinephrine, could be used to allow self-administration of vaccine. The use of such delivery devices may be particularly amenable to large scale immunization campaigns.

Intradermal delivery is another suitable route. Any suitable device may be used for intradermal delivery, for example short needle devices. Such devices are well known in the art. Intradermal vaccines may also be administered by devices which limit the effective penetration length of a needle into the skin, such as those described in WO99/34850 and EP1092444, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Also suitable, are ballistic powder/particle delivery devices which use compressed gas to accelerate vaccine in powder form through the outer layers of the skin to the dermis. Additionally, conventional syringes may be used in the classical mantoux method of intradermal administration.

Another suitable administration route is the subcutaneous route. Any suitable device may be used for subcutaneous delivery, for example classical needle. Suitably, a needle-free jet injector service is used. Such devices are well known in the art. Suitably said device is pre-filled with the liquid vaccine formulation.

Alternatively the vaccine is administered intranasally. Typically, the vaccine is administered locally to the nasopharyngeal area, suitably without being inhaled into the lungs. It is desirable to use an intranasal delivery device which delivers the vaccine formulation to the nasopharyngeal area, without or substantially without it entering the lungs.

Suitable devices for intranasal administration of the vaccines according to the invention are spray devices. Suitable commercially available nasal spray devices include Accuspray™ (Becton Dickinson). Nebulisers produce a very fine spray which can be easily inhaled into the lungs and therefore does not efficiently reach the nasal mucosa. Nebulisers are therefore not preferred.

Suitable spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO 91/13281 and EP 311 863 B and EP 516 636, incorporated herein by reference. Such devices are commercially available from Pfeiffer GmbH and are also described in Bommer, R. Pharmaceutical Technology Europe, Sept 1999.

Alternatively, the epidermal or transdermal vaccination route is also contemplated herein.

Sequences

SEQ ID NO: 1 Full length HA sequence from A/Brisbane/02/2018 (H1N1)pdm09-like virus
(H1_{Bri18}) also referred to as Bri18.
MKAILVVLLYTFTTANADTLCIGYHANNSTDTVDTVLEKNVTVTHSVNLLEDKHNGKLCKLGGVAPLHLGKCNIAG
WILGNPECESLSTARSWSYIVETSNSDNGTCYPGDFINYEELREQLSSVSSFERFEIFPKTSSWPNHDSNKGVTAACP
HAGAKSFYKNLIWLVKKGNSYPKLNQTYINDKGKEVLVLWGIHHPPTTADQQSLYQNADAYVFVGTSRYSKKFKPE
IATRPKVRDQEGRMNYYWTLVEPGDKITFEATGNLVVPRYAFTMERNAGSGIIISDTPVHDCNTTCQTAEGAINTS
LPFQNVHPVTIGKCPKYVKSTKLRLATGLRNVPSIQSRGLFGAIAGFIEGGWTGMVDGWYGYHHQNEQGSGYAA
DLKSTQNAIDKITNKVNSVIEKMNTQFTAVGKEFNHLEKRIENLNKKVDDGFLDIWTYNAELLVLLENERTLDYHDS
NVKNLYEKVRNQLKNNAKEIGNGCFEFYHKCDNTCMESVKNGTYDYPKYSEEAKLNREKIDGVKLESTRIYQILAIYS
TVASSLVLVVSLGAISFWMCSNGSLQCRICI
SEQ ID NO: 2 Full length HA sequence from A/Darwin/9/2021 H3N2 also referred to as Darw21
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALENQHTIDLTDSEMNKLFEKTKKQLRENAEDMGN
GCFKIYHKCDNACIGSIRNETYDHNVYRDEALNNRFQIKGVELKSGYKDWILWISFAMSCFLLCIALLGFIMWACQKGNI
RCNICI
SEQ ID NOS: 3-8 show in bold mutation(s) compared to H1 Brisbane 18 WT sequence:
SEQ ID NO: 3 Mut10
MKAILVVLLYTFTTANADTLCIGYHANNSTDTVDTVLEKNVTVTHSVNLLEDKHNGKLCKLGGVAPLHLGKCNIA
GWILGNPECESLSTARSWSYIVETSNSDNGTCYPGDFINYEELREQLSSVSSFERFEIFPKTSSWPNHDSNKGVT
AACPHAGAKSFYKNLIWLVKKGNSYPKLNQTYINDKGKEVLVLWGIHHPPTTADQQSLYQNADAYVFVGTSRYSK
KFKPEIATRPKVRDQEGRMNYYWTLVEPGDKITFEATGNLVVPRYAFTMERNAGSGIIISDTPVHDCNTTCQTAE
GAINTSLPFQNVHPVTIGKCPKYVKSTKLRLATGLRNVPSIQSRGLFGAIAGFIEGGWTGMVDGWYGYHHQNEQG
SGYAADLKSTQNAIDKITNMVNSVIEKMNTQFTAVGKEFNHLEKRIENLNKKVDDGFLDIWTYNAELLVLLENER
TLDYHDSNVKNLYEKVRNQLKNNAKEIGNGCFEFYHKCDNTCMESVKNGTYDYPKYSEEAKLNREKIDGVGYIPE
APRDGQAYVRKDGEWVLLSTFLGHHHHHH
SEQ ID NO: 4 Mut17
MKAILVVLLYTFTTANADTLCIGYHANNSTDTVDTVLEKNVTVTHSVNLLEDKHNGKLCKLGGVAPLHLGKCNIA
GWILGNPECESLSTARSWSYIVETSNSDNGTCYPGDFINYEELREQLSSVSSFERFEIFPKTSSWPNHDSNKGVT
AACPHAGAKSFYKNLIWLVKKGNSYPKLNQTYINDKGKEVLVLWGIHHPPTTADQQSLYQNADAYVFVGTSRYSK
KFKPEIATRPKVRDQEGRMNYYWTLVEPGDKITFEATGNLVVPRYAFTMERNAGSGIIISDTPVHDCNTTCQTAE
GAINTSLPFQNVHPVTIGKCPKYVKSTKLRLATGLRNVPSIQSRGLFGAIAGFIEGGWTGMVDGWYGYHHQNEQG
SGYAADLKSTQNAIDKITNMVNSVIEKMNTQFTAVGKEFNHLEKRIENLNKKVDDGFLDIWTYNAELLVLLLNQW
TLLYHDSNVKNLYEKVRNQLKNNAKEIGNGCFEFYHKCDNTCMESVKNGTYDYPKYSEEAKLNREKIDGVGSENL
YFQGGSKGYIPEAPRDGQAYVRKDGEWVLLSTFLGHHHHHH
SEQ ID NO: 5 Mut18
MKAILVVLLYTFTTANADTLCIGYHANNSTDTVDTVLEKNVTVTHSVNLLEDKHNGKLCKLGGVAPLHLGKCNIA
GWILGNPECESLSTARSWSYIVETSNSDNGTCYPGDFINYEELREQLSSVSSFERFEIFPKTSSWPNHDSNKGVT
AACPHAGAKSFYKNLIWLVKKGNSYPKLNQTYINDKGKEVLVLWGIHHPPTTADQQSLYQNADAYVFVGTSRYSK
KFKPEIATRPKVRDQEGRMNYYWTLVEPGDKITFEATGNLVVPRYAFTMERNAGSGIIISDTPVHDCNTTCQTAE
GAINTSLPFQNVHPVTIGKCPKYVKSTKLRLATGLRNVPSIQSRGLFGAIAGFIEGGWTGMVDGWYGYHHQNEQG
SGYAADLKSTQNAIDKITNKVNSVIEKMNTQFTAVGKEFNHLEKRIENLNKKVDDGFLDIWTYNAELLVLLENER
TLDYHDSNVINLYEKVRNQLKNNAKEIGNGCFEFYHKCDNTCMESVKNGTYDYPKYSEEAKLNREKIDGVGSENL
YFQGGSKGYIPEAPRDGQAYVRKDGEWVLLSTFLGHHHHHH
SEQ ID NO: 6 Mut23
MKAILVVLLYTFTTANADTLCIGYHANNSTDTVDTVLEKNVTVTHSVNLLEDKHNGKLCKLGGVAPLHLGKCNIA
GWILGNPECESLSTARSWSYIVETSNSDNGTCYPGDFINYEELREQLSSVSSFERFEIFPKTSSWPNHDSNKGVT
AACPHAGAKSFYKNLIWLVKKGNSYPKLNQTYINDKGKEVLVLWGIHHPPTTADQQSLYQNADAYVFVGTSRYSK
KFKPEIATRPKVRDQEGRMNYYWTLVEPGDKITFEATGNLVVPRYAFTMERNAGSGIIISDTPVHDCNTTCQTAE
GAINTSLPFQNVHPVTIGKCPRYVKSTKLRLATGLRNVPSIQSRGLFGAIAGFIEGGWTGMVDGWYGYHHQNEQG
SGYAADLKSTQNAIDKITNMVNSVIEKMNTQFTAVGKEFNHLEKRIENLNKKVDDGFLDIDTYNAELLVLLLNER
TLDYHDSNVKNLYEKVRNQLKNNAKEIGNGCFEFYHKCDNTCMESVKNGTYDYPKYSEEAKLNREKIDGVGSENL
YFQGGSKGYIPEAPRDGQAYVRKDGEWVLLSTFLGHHHHHH
SEQ ID NO: 7 Mut24
MKAILVVLLYTFTTANADTLCIGYHANNSTDTVDTVLEKNVTVTHSVNLLEDKHNGKLCKLGGVAPLHLGKCNIA
GWILGNPECESLSTARSWSYIVETSNSDNGTCYPGDFINYEELREQLSSVSSFERFEIFPKTSSWPNHDSNKGVT
AACPHAGAKSFYKNLIWLVKKGNSYPKLNQTYINDKGKEVLVLWGIHHPPTTADQQSLYQNADAYVFVGTSRYSK
KFKPEIATRPKVRDQEGRMNYYWTLVEPGDKITFEATGNLVVPRYAFTMERNAGSGIIISDTPVHDCNTTCQTAE
GAINTSLPFQNVHPVTIGKCPRYVKSTKLRLATGLRNVPSIQSRGLFGAIAGFIEGGWTGMVDGWYGYHHQNEQG
SGYAADLKSTQNAIDKITNKVNSVIEKMNTQFTAVGKEFNHLEKRIENLNKKVDDGFLDIDTYLAELLVLLENQW
TLLYHDSNVKNLYEKVRNQLKNNAKEIGNGCFEFYHKCDNTCMESVKNGTYDYPKYSEEAKLNREKIDGVGSENL
YFQGGSKGYIPEAPRDGQAYVRKDGEWVLLSTFLGHHHHHH
SEQ ID NO: 8 Mut27
MKAILVVLLYTFTTANADTLCIGYHANNSTDTVDTVLEKNVTVTHSVNLLEDKHNGKLCKLGGVAPLHLGKCNIA
GWILGNPECESLSTARSWSYIVETSNSDNGTCYPGDFINYEELREQLSSVSSFERFEIFPKTSSWPNHDSNKGVT
AACPHAGAKSFYKNLIWLVKKGNSYPKLNQTYINDKGKEVLVLWGIHHPPTTADQQSLYQNADAYVFVGTSRYSK
KFKPEIATRPKVRDQEGRMNYYWTLVEPGDKITFEATGNLVVPRYAFTMERNAGSGIIISDTPVHDCNTTCQTAE
GAINTSLPFQNVHPVTIGKCPRYVKSTKLRLATGLRNVPSIQSRGLFGAIAGFIEGGWTGMVDGWYGYHHQNEQG
SGYAADLKSTQNAIDKITNMVNSVIEKMNTQFTAVGKEFNHLEKRIENLNKKVDDGFLDIDTYLAELLVLLLNQW
TLLYHDSNVKNLYEKVRNQLKNNAKEIGNGCFEFYHKCDNTCMESVKNGTYDYPKYSEEAKLNREKIDGVGSENL
YFQGGSKGYIPEAPRDGQAYVRKDGEWVLLSTFLGHHHHHH
SEQ ID NO: 9 Foldon sequence
GYIPEAPRDGQAYVRKDGEWVLLSTFL
SEQ ID NO: 10 Bri 18 Signal peptide
MKAILVVLLYTFTTANA
SEQ ID NOS: 11-16 show nucleotide sequences encoding the amino acid sequences of SEQ ID
NOS: 3-8
SEQ ID NO: 11 Mut10
ATGAAAGCTATCCTCGTGGTGCTGCTGTACACCTTCACCACCGCCAACGCTGACACACTGTGCATCGGATACCATGCCAACAA
TTCCACCGACACAGTGGATACCGTGCTGGAGAAAAACGTGACCGTCACCCACTCCGTCAATCTGCTCGAGGACAAGCACAACG
GAAAGCTCTGCAAGCTGGGCGGAGTGGCTCCTCTCCATCTGGGAAAGTGCAACATTGCCGGCTGGATTCTGGGAAACCCCGAG
TGCGAGTCTCTGTCCACCGCTAGGAGCTGGAGCTATATCGTCGAAACCTCCAACTCCGACAACGGCACATGCTACCCCGGCGA
CTTCATTAACTACGAGGAACTGAGAGAGCAGCTCAGCTCCGTGTCCAGCTTTGAGAGGTTTGAGATCTTCCCCAAGACCAGCA
GCTGGCCCAACCACGACAGCAACAAAGGCGTCACCGCCGCTTGCCCTCACGCTGGCGCCAAATCCTTCTACAAAAATCTGATC
TGGCTCGTCAAGAAGGGCAATAGCTACCCCAAGCTCAACCAGACCTACATCAACGACAAAGGCAAAGAGGTGCTCGTCCTCTG
GGGAATCCACCACCCTCCTACCACAGCCGACCAGCAGTCTCTGTACCAGAACGCCGACGCCTATGTCTTTGTGGGAACCTCTA
GGTACTCCAAGAAGTTCAAGCCCGAGATCGCCACAAGGCCTAAAGTGAGAGACCAAGAGGGAAGGATGAACTACTACTGGACA
CTGGTGGAACCCGGCGATAAGATCACCTTTGAGGCCACCGGCAACCTCGTGGTCCCCAGATACGCTTTCACCATGGAGAGGAA
CGCCGGCAGCGGAATCATCATCAGCGATACACCCGTGCACGACTGTAACACCACATGCCAGACCGCCGAGGGCGCCATTAACA
CCAGCCTCCCCTTCCAGAACGTGCACCCCGTCACAATCGGCAAGTGCCCCAAGTACGTGAAGAGCACAAAGCTGAGACTCGCT
ACCGGACTGAGGAACGTGCCTTCCATCCAAAGCAGAGGACTGTTTGGAGCCATCGCCGGCTTCATCGAAGGAGGCTGGACCGG
CATGGTGGATGGATGGTACGGCTACCACCACCAAAACGAACAAGGCTCCGGATACGCCGCCGACCTCAAGTCCACCCAGAATG
CCATCGACAAAATCACCAACATGGTGAACTCCGTGATCGAGAAGATGAACACCCAGTTCACCGCTGTGGGCAAGGAGTTCAAC
CACCTCGAGAAGAGGATCGAAAACCTCAACAAGAAAGTGGACGACGGATTTCTGGACATCTGGACCTATAACGCCGAACTGCT
CGTGCTGCTGGAAAACGAGAGAACACTGGACTACCACGACTCCAATGTGAAGAATCTGTACGAGAAGGTGAGGAACCAGCTGA
AGAACAACGCCAAGGAGATCGGCAACGGCTGCTTCGAGTTCTACCACAAATGCGACAACACATGCATGGAGTCCGTCAAGAAC
GGAACCTACGACTATCCCAAGTACAGCGAGGAGGCCAAACTGAACAGAGAGAAGATTGACGGCGTGGGCTACATCCCCGAGGC
TCCTAGAGACGGCCAAGCCTACGTGAGGAAGGACGGAGAGTGGGTGCTGCTGTCCACCTTTCTGGGACACCACCATCATCACC
ACTGATGA
SEQ ID NO: 12 Mut17
ATGAAGGCTATTCTGGTGGTGCTGCTGTACACCTTCACCACCGCCAATGCCGACACACTGTGCATTGGCTACCACGCCAACAA
CAGCACCGATACAGTGGACACAGTCCTCGAGAAGAACGTGACCGTCACACACAGCGTGAATCTGCTCGAGGACAAGCACAACG
GAAAGCTCTGTAAGCTGGGAGGAGTCGCCCCTCTGCATCTGGGCAAGTGTAACATCGCCGGCTGGATTCTGGGCAACCCCGAG
TGTGAATCTCTGTCCACCGCCAGAAGCTGGAGCTACATCGTGGAAACAAGCAACAGCGACAACGGCACATGCTACCCCGGCGA
CTTCATCAACTACGAAGAGCTGAGAGAGCAGCTGTCCAGCGTCAGCAGCTTCGAGAGATTCGAGATCTTTCCCAAGACCAGCA
GCTGGCCCAATCACGATTCCAACAAGGGAGTCACCGCTGCTTGCCCCCACGCTGGCGCTAAGTCCTTTTACAAGAACCTCATT
TGGCTCGTGAAGAAGGGCAACTCCTATCCCAAGCTCAATCAGACATATATTAACGATAAAGGAAAGGAAGTGCTGGTGCTCTG
GGGCATCCACCACCCTCCCACCACCGCTGACCAACAGAGCCTCTACCAGAATGCTGACGCCTATGTCTTTGTGGGAACCTCTA
GGTACTCCAAGAAGTTCAAGCCCGAGATTGCTACAAGACCCAAGGTGAGGGACCAAGAGGGAAGAATGAACTACTACTGGACA
CTGGTGGAGCCCGGCGATAAGATTACCTTCGAGGCCACCGGCAATCTGGTCGTCCCCAGATATGCCTTCACCATGGAGAGAAA
CGCCGGCAGCGGAATCATTATCAGCGACACCCCCGTGCATGATTGCAACACAACATGCCAAACCGCCGAGGGCGCCATTAACA
CCTCCCTCCCCTTTCAAAACGTCCACCCCGTGACAATCGGCAAGTGCCCCAAGTACGTGAAATCCACCAAGCTGAGGCTGGCC
ACCGGACTGAGAAACGTGCCCTCCATTCAGAGCAGAGGACTGTTTGGAGCCATTGCCGGCTTTATTGAGGGCGGCTGGACCGG
AATGGTCGATGGCTGGTACGGCTATCATCACCAAAACGAGCAAGGCTCCGGATATGCTGCTGATCTGAAGTCCACACAAAACG
CCATCGATAAGATCACCAACATGGTGAACAGCGTGATCGAGAAGATGAACACCCAGTTCACAGCCGTGGGCAAGGAATTTAAC
CACCTCGAGAAAAGGATCGAGAACCTCAACAAAAAGGTCGACGACGGCTTCCTCGACATTTGGACCTACAACGCTGAGCTGCT
CGTGCTGCTGCTGAACCAGTGGACACTGCTCTATCACGACTCCAACGTGAAGAATCTGTATGAGAAGGTGAGAAACCAACTGA
AGAACAACGCTAAGGAGATCGGAAACGGCTGCTTCGAGTTTTACCACAAGTGCGACAACACATGCATGGAGTCCGTGAAAAAC
GGCACATACGACTACCCCAAGTATTCCGAGGAAGCCAAGCTCAATAGAGAAAAGATCGATGGCGTCGGCAGCGAGAATCTGTA
TTTTCAAGGCGGCTCCAAGGGCTATATTCCCGAGGCTCCTAGAGACGGACAAGCCTATGTGAGGAAGGATGGCGAATGGGTGC
TCCTCTCCACATTTCTGGGCCACCACCACCATCATCACTGATGA
SEQ ID NO: 13 Mut18
ATGAAGGCTATTCTGGTCGTGCTGCTGTACACCTTCACCACAGCCAACGCCGATACACTGTGTATCGGCTACCACGCCAACAA
CAGCACCGATACAGTCGATACCGTGCTGGAGAAGAACGTGACCGTCACCCATTCCGTGAATCTGCTGGAGGACAAGCACAATG
GCAAGCTGTGCAAGCTGGGCGGAGTGGCCCCTCTGCATCTGGGCAAGTGTAATATCGCCGGATGGATTCTGGGAAACCCCGAG
TGTGAATCTCTGTCCACCGCTAGAAGCTGGTCCTACATTGTGGAGACCTCCAATTCCGACAATGGCACATGCTACCCCGGCGA
TTTTATCAACTACGAGGAGCTGAGAGAACAACTGAGCAGCGTCTCCTCCTTTGAGAGATTCGAGATCTTTCCCAAGACCAGCA
GCTGGCCCAATCACGATTCCAACAAGGGAGTCACCGCTGCTTGCCCCCACGCTGGCGCTAAGTCCTTTTACAAGAACCTCATT
TGGCTCGTGAAGAAGGGCAACTCCTATCCCAAGCTGAATCAAACATATATCAACGACAAGGGCAAGGAAGTCCTCGTGCTGTG
GGGAATCCACCACCCTCCTACAACAGCCGACCAACAGTCTCTGTATCAGAACGCCGACGCCTATGTGTTCGTGGGAACCTCTA
GGTACTCCAAGAAGTTCAAGCCCGAGATTGCTACAAGGCCCAAGGTGAGAGACCAAGAGGGAAGAATGAATTACTACTGGACA
CTGGTGGAGCCCGGCGATAAGATCACCTTCGAAGCCACCGGCAACCTCGTGGTGCCTAGGTACGCCTTCACCATGGAGAGAAA
CGCTGGCAGCGGCATCATCATCTCCGACACACCCGTGCATGACTGCAACACAACATGCCAAACAGCCGAGGGCGCCATTAACA
CCTCCCTCCCCTTTCAGAACGTGCACCCCGTGACAATCGGCAAGTGCCCCAAGTACGTGAAATCCACAAAGCTGAGGCTGGCC
ACCGGACTGAGAAATGTGCCCAGCATCCAGTCTAGGGGACTGTTTGGAGCTATCGCCGGCTTCATTGAAGGCGGCTGGACCGG
AATGGTGGACGGATGGTACGGCTACCACCACCAGAATGAACAAGGCTCCGGATACGCTGCTGATCTGAAGAGCACCCAAAACG
CCATTGACAAGATCACCAACAAGGTCAACTCCGTCATCGAGAAGATGAACACCCAGTTTACCGCCGTGGGCAAGGAATTTAAC
CACCTCGAGAAAAGGATCGAGAACCTCAACAAAAAGGTCGACGACGGCTTTCTCGACATTTGGACCTACAACGCCGAGCTGCT
GGTGCTGCTCGAGAATGAGAGAACACTGGACTACCACGACTCCAATGTCATCAATCTGTATGAGAAGGTGAGGAACCAGCTCA
AGAACAACGCTAAGGAGATCGGAAACGGCTGCTTCGAGTTTTATCACAAGTGCGATAACACATGCATGGAGTCCGTGAAAAAC
GGCACATACGACTATCCCAAGTACTCCGAAGAGGCTAAGCTCAATAGAGAGAAGATCGATGGCGTCGGCAGCGAGAATCTCTA
TTTCCAAGGCGGCAGCAAGGGCTATATCCCCGAGGCCCCTAGAGATGGCCAAGCCTATGTGAGGAAGGACGGCGAATGGGTGC
TGCTGAGCACCTTCCTCGGCCATCACCATCACCACCACTGATGA
SEQ ID NO: 14 Mut23
ATGAAAGCCATTCTGGTGGTGCTGCTGTACACCTTTACCACCGCCAATGCCGATACACTGTGCATCGGCTACCACGCCAATAA
CTCCACCGACACCGTGGACACAGTGCTGGAGAAGAACGTGACCGTGACACACAGCGTGAACCTCCTCGAGGACAAGCACAACG
GAAAGCTGTGCAAGCTCGGCGGAGTGGCCCCTCTGCATCTGGGAAAGTGTAACATTGCCGGCTGGATTCTGGGAAACCCCGAG
TGTGAATCTCTGAGCACCGCTAGAAGCTGGAGCTACATTGTGGAGACCAGCAACAGCGACAACGGAACATGCTACCCCGGCGA
CTTTATCAATTACGAGGAACTGAGGGAGCAGCTGTCCTCCGTGAGCTCCTTTGAGAGGTTCGAGATCTTTCCCAAGACCAGCA
GCTGGCCCAATCACGACTCCAACAAAGGAGTGACCGCTGCTTGCCCTCACGCCGGCGCTAAGAGCTTCTACAAAAACCTCATC
TGGCTGGTGAAGAAGGGCAACTCCTACCCCAAGCTGAACCAAACCTACATCAATGACAAAGGCAAGGAGGTCCTCGTGCTCTG
GGGAATCCACCACCCTCCCACAACCGCTGACCAACAGAGCCTCTACCAGAACGCTGACGCCTATGTCTTTGTGGGAACCTCTA
GGTACTCCAAGAAGTTCAAGCCCGAGATTGCTACAAGGCCCAAGGTGAGAGACCAAGAGGGAAGAATGAATTACTACTGGACA
CTCGTGGAGCCCGGCGACAAAATCACCTTCGAAGCCACCGGCAACCTCGTGGTCCCCAGATACGCCTTCACCATGGAGAGGAA
CGCTGGCTCCGGCATCATCATTAGCGACACACCCGTGCACGACTGCAATACAACATGCCAAACAGCTGAGGGCGCCATCAATA
CCTCTCTGCCCTTTCAGAACGTGCACCCCGTGACAATCGGCAAATGCCCTAGGTACGTGAAGAGCACCAAGCTCAGACTCGCC
ACCGGACTGAGGAACGTGCCCTCCATCCAGTCTAGGGGACTGTTTGGAGCCATTGCCGGCTTTATTGAGGGCGGCTGGACCGG
AATGGTGGACGGATGGTACGGCTACCACCACCAGAATGAACAAGGCTCCGGATACGCCGCTGATCTGAAGAGCACACAGAATG
CCATTGATAAGATTACCAACATGGTGAACAGCGTGATCGAGAAGATGAACACCCAGTTCACCGCCGTCGGCAAGGAATTTAAC
CACCTCGAGAAAAGGATCGAGAACCTCAACAAAAAGGTCGACGACGGCTTCCTCGACATTGACACCTACAACGCTGAGCTGCT
GGTGCTGCTGCTGAATGAGAGAACACTGGACTACCACGATAGCAATGTCAAGAATCTGTATGAAAAGGTGAGAAACCAACTGA
AGAACAACGCCAAGGAGATCGGAAACGGCTGCTTCGAGTTTTATCACAAGTGCGATAACACATGCATGGAGTCCGTGAAAAAC
GGCACCTACGACTATCCCAAGTACTCCGAGGAGGCTAAGCTCAATAGAGAAAAGATCGACGGCGTCGGAAGCGAAAACCTCTA
CTTTCAAGGCGGCAGCAAGGGCTATATCCCCGAGGCTCCTAGAGATGGACAAGCCTATGTGAGGAAGGACGGCGAATGGGTCC
TCCTCAGCACCTTTCTGGGACACCACCACCATCACCACTGATGA
SEQ ID NO: 15 Mut24
ATGAAGGCTATTCTGGTCGTGCTGCTCTACACCTTTACCACCGCCAATGCCGATACACTGTGCATCGGCTATCACGCCAACAA
CAGCACCGATACCGTCGACACCGTGCTGGAGAAGAACGTGACCGTCACACACAGCGTGAATCTGCTGGAGGACAAGCACAATG
GCAAGCTGTGCAAGCTGGGCGGAGTGGCCCCTCTGCATCTGGGCAAGTGTAATATTGCCGGATGGATTCTGGGCAACCCCGAG
TGCGAGTCTCTGAGCACCGCTAGAAGCTGGAGCTACATTGTGGAGACCAGCAACTCCGACAATGGCACATGCTACCCCGGCGA
TTTTATCAACTACGAGGAACTGAGGGAGCAGCTCAGCTCCGTCTCCAGCTTCGAGAGATTCGAAATCTTCCCCAAGACATCCA
GCTGGCCCAACCATGACTCCAACAAGGGAGTGACAGCTGCTTGCCCTCACGCTGGCGCCAAAAGCTTCTACAAGAACCTCATT
TGGCTGGTGAAGAAGGGCAACTCCTATCCCAAGCTGAATCAAACATATATCAACGACAAGGGCAAGGAAGTGCTGGTGCTGTG
GGGCATTCACCACCCTCCCACCACCGCTGATCAGCAGAGCCTCTACCAGAATGCCGACGCCTATGTGTTTGTGGGAACCTCTA
GGTACTCCAAGAAGTTCAAGCCCGAGATCGCCACAAGGCCCAAGGTGAGAGACCAAGAGGGAAGAATGAACTACTACTGGACA
CTGGTGGAGCCCGGCGATAAGATTACCTTCGAGGCCACCGGCAATCTGGTCGTCCCCAGATATGCCTTCACCATGGAGAGAAA
CGCCGGCAGCGGAATCATTATCTCCGACACCCCCGTGCACGATTGCAACACCACATGCCAGACCGCCGAGGGCGCCATCAACA
CAAGCCTCCCTTTTCAGAACGTGCACCCCGTGACAATCGGCAAGTGTCCTAGGTACGTCAAGAGCACCAAGCTCAGACTGGCC
ACCGGACTGAGGAACGTGCCCTCCATCCAGTCTAGGGGACTGTTTGGAGCCATTGCCGGCTTTATTGAGGGAGGATGGACCGG
CATGGTCGATGGCTGGTACGGCTACCACCATCAGAATGAACAAGGCTCCGGATACGCTGCCGACCTCAAGAGCACACAGAATG
CCATCGACAAGATCACAAATAAAGTCAACAGCGTGATCGAGAAGATGAACACCCAGTTTACCGCCGTCGGCAAAGAGTTCAAC
CACCTCGAGAAGAGGATCGAGAACCTCAACAAGAAGGTGGACGACGGCTTTCTGGATATCGACACCTATCTGGCTGAGCTGCT
GGTGCTGCTCGAGAATCAGTGGACACTGCTGTATCATGATTCCAACGTCAAGAATCTGTACGAGAAGGTGAGAAACCAACTGA
AGAACAACGCCAAGGAGATCGGAAACGGCTGCTTCGAGTTTTACCACAAGTGCGATAATACATGCATGGAGAGCGTCAAGAAC
GGCACCTACGACTATCCCAAGTACTCCGAAGAGGCTAAGCTCAATAGAGAAAAGATCGACGGCGTCGGCAGCGAGAATCTCTA
TTTCCAAGGCGGCTCCAAGGGCTATATTCCCGAGGCTCCTAGAGACGGACAAGCCTATGTGAGGAAGGATGGCGAATGGGTGC
TCCTCTCCACATTCCTCGGACACCACCATCACCACCACTGATGA
SEQ ID NO: 16 Mut27
ATGAAGGCTATTCTGGTCGTCCTCCTCTACACCTTCACCACCGCTAATGCCGACACACTGTGCATCGGCTACCATGCCAACAA
CAGCACCGATACAGTGGACACCGTGCTGGAGAAAAACGTGACCGTCACACACAGCGTGAATCTGCTGGAGGACAAGCACAATG
GCAAGCTGTGCAAGCTGGGCGGAGTGGCCCCTCTGCATCTCGGCAAGTGTAACATCGCTGGATGGATTCTGGGCAATCCCGAG
TGCGAGTCTCTGAGCACAGCTAGAAGCTGGAGCTACATTGTGGAGACCAGCAACAGCGACAACGGCACATGTTACCCCGGAGA
CTTCATCAATTACGAGGAGCTGAGAGAACAGCTCAGCTCCGTGTCCAGCTTTGAGAGATTCGAGATCTTCCCCAAGACCTCCA
GCTGGCCCAACCACGACAGCAATAAGGGAGTGACCGCTGCTTGTCCCCACGCTGGCGCCAAGAGCTTCTACAAAAACCTCATC
TGGCTGGTGAAGAAGGGCAACTCCTACCCCAAACTCAACCAGACCTATATTAACGATAAAGGAAAGGAAGTGCTGGTGCTCTG
GGGCATCCACCACCCTCCCACCACCGCTGACCAACAGAGCCTCTACCAGAATGCTGACGCCTATGTCTTTGTGGGAACCTCTA
GGTACTCCAAGAAGTTCAAGCCCGAGATTGCTACAAGGCCCAAGGTGAGAGACCAAGAGGGAAGAATGAATTACTACTGGACA
CTGGTGGAGCCCGGCGACAAAATCACCTTCGAAGCCACCGGCAACCTCGTGGTCCCCAGATACGCCTTCACCATGGAGAGGAA
CGCTGGCTCCGGAATCATCATTTCCGACACCCCCGTGCACGACTGCAATACAACATGCCAGACCGCCGAGGGCGCCATCAATA
CCTCTCTGCCCTTCCAGAATGTGCACCCCGTGACCATCGGCAAGTGTCCTAGGTACGTCAAGAGCACCAAGCTGAGACTGGCC
ACCGGACTGAGGAACGTGCCCTCCATCCAGTCTAGGGGACTGTTCGGAGCCATTGCCGGATTCATCGAGGGCGGCTGGACCGG
AATGGTGGACGGATGGTACGGCTACCACCACCAAAACGAGCAAGGCTCCGGATACGCCGCTGATCTGAAGAGCACACAGAACG
CCATCGACAAGATCACCAATATGGTCAACAGCGTGATTGAAAAGATGAACACCCAGTTCACCGCCGTGGGAAAAGAGTTCAAC
CATCTCGAGAAGAGGATCGAGAACCTCAACAAGAAGGTGGACGATGGCTTCCTCGACATCGATACCTATCTGGCTGAGCTGCT
GGTGCTGCTGCTCAATCAATGGACACTGCTGTACCACGACTCCAACGTCAAGAATCTGTACGAGAAGGTGAGAAATCAGCTCA
AAAACAACGCCAAAGAGATCGGCAACGGCTGCTTCGAGTTTTATCACAAGTGCGATAACACATGCATGGAGTCCGTGAAGAAC
GGCACCTACGACTATCCCAAGTACTCCGAAGAGGCCAAGCTCAATAGAGAGAAGATTGACGGAGTGGGCTCCGAGAACCTCTA
CTTCCAAGGCGGCTCCAAAGGCTACATCCCCGAGGCCCCTAGAGATGGACAAGCCTACGTGAGAAAGGACGGAGAGTGGGTGC
TCCTCAGCACATTTCTGGGACATCATCATCACCACCACTGATGA
SEQ ID NOS: 17-42 show HA polypeptide antigens containing mutation(s)
compared to H1 Brisbane 18 WT sequence
SEQ ID NO: 17
>Flu622
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNLL
IGKTNEKFHQIEKEFSEVEGRVQDLEKYVEDTKIDLWSYNAELLVALMNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 18
>Flu629
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNML
IGKTNEKFHQIEKEFSEVEGRVQDLEKYVEDTKIDLWSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 19
>Flu632
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGILNFL
IGKTNEKFHQIEKEFSEVEGRVQDLEKYVEDTKIDLWSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 20
>Flu638
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGVLNIL
IGKTNEKFHQIEKEFSEVEGRVQDLEKYVEDTKIDLWSYNAELLVALMNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 21
>Flu639
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGKLNRL
IGKTNEKFHQIEKEFSEPEGRVQDLEKYVEDTKIDLWSYNAELLVALENQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 22
>Flu643
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGKLNRL
IGKTNEKFHQIEKEFSEVEGRVQDLEKYVEDTKIDLWSYIAELLVALENQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 23
>Flu650
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGFFGAIAGFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALENQLTIALTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 24
>Flu672
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALENQHTIDLTDSEMNKLFMKTMKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 25
>Flu679
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNLL
IGKTNEKFHQIEKEFSEVEGRVQDLEKYVEDTKIDLDSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 26
>Flu680
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNLL
IGKTNEKFHQIEKEFSEVEGRVQDLEKYVEDTKIDLDSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 27
>Flu681
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNLL
IGKTNEKFHQIEKEFSEVEGRVQDLEKYVEDTKIDLDSYNAELLVALMNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 28
>Flu682
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGVLNLL
IGKTNEKFHQIEKEFSEVEGRVQDLEKYVEDTKIDLDSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 29
>Flu683
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGVLNLL
IGKTNEKFHQIEKEFSEVEGRVQDLEKYVEDTKIDLDSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 30
>Flu685
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGMLNLL
IGKTNEKFHQIEKEFSEVEGRVQDLEKYVEDTKIDLDSYNAELLVALVNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 31
>Flu686
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGMLNLL
IGKTNEKFHQIEKEFSEVEGRVQDLEKYVEDTKIDLDSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 32
>Flu687
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGMLNLL
IGKTNEKFHQIEKEFSEVEGRVQDLEKYVEDTKIDLDSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 33
>Flu688
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNML
IGKTNEKFHQIEKEFSEVEGRVQDLEKYVEDTKIDLDSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 34
>Flu689
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNFL
IGKTNEKFHQIEKEFSEVEGRVQDLEKYVEDTKIDLDSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 35
>Flu690
MKTIIALSNILCLVFAQKIPGNDNSTATLCLGHHAVPNGTIVKTITNDRIEVTNATELVONSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNFL
IGKTNEKFHQIEKEFSEVEGRVQDLEKYVEDTKIDLDSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 36
>Flu691
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGILNFL
IGKTNEKFHQIEKEFSEVEGRVQDLEKYVEDTKIDLDSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 37
>Flu692
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNIL
IGKTNEKFHQIEKEFSEVEGRVQDLEKYVEDTKIDLDSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 38
>Flu693
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGLLNIL
IGKTNEKFHQIEKEFSEVEGRVQDLEKYVEDTKIDLDSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 39
>Flu695
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGVLNIL
IGKTNEKFHQIEKEFSEVEGRVQDLEKYVEDTKIDLDSYNAELLVALLNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 40
>Flu696
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGVLNIL
IGKTNEKFHQIEKEFSEVEGRVQDLEKYVEDTKIDLDSYNAELLVALINQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 41
>Flu697
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGVLNIL
IGKTNEKFHQIEKEFSEVEGRVQDLEKYVEDTKIDLDSYNAELLVALMNQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 42
>Flu707
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGKLNRL
IGKTNEKFHQIEKEFSEPEGRVQDLEKYVEDTKIDLDSYNAELLVALENQHTIDLTDSEMNKLFEKTKKQLRENAEDMGN
GCFKIYHKCDNACIGSIRNETYDHNVYRDEALNNRFQIKGVGSENLYFQGGSKGYIPEAPRDGQAYVRKDGEWVLLSTFL
GHHHHHH
SEQ ID NO: 43
Darw21 Signal peptide
MKTIIALSNILCLVFA
SEQ ID NOS: 44-69 show nucleotide sequences encoding the amino acid sequences of SEQ ID
NOS: 17-42
SEQ ID NO: 44
>Flu622
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGTGGAGCTACAACGCCGAGCT
GCTGGTGGCCCTGATGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 45
>Flu629
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACATGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGTGGAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 46
>Flu632
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCATCCTGAACTTCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGTGGAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 47
>Flu638
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCGTGCTGAACATCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGTGGAGCTACAACGCCGAGCT
GCTGGTGGCCCTGATGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 48
>Flu639
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCAAGCTGAACAGACTGATCGGCAAGACCAACGAGAAGTTCCATCAGATCGAGAAGGAGTTC
AGCGAGCCCGAGGGCAGAGTGCAAGACCTGGAGAAGTACGTGGAGGACACCAAGATCGACCTGTGGAGCTACAACGCCGAGCT
GCTGGTGGCCCTGGAGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 49
>Flu643
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCAAGCTGAACAGACTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGTGGAGCTACATCGCCGAGCT
GCTGGTGGCCCTGGAGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 50
>Flu650
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCTTCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCAAGCTGAACAGACTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGTGGAGCTACAACGCCGAGCT
GCTGGTGGCCCTGGAGAATCAGCTGACCATCGCCCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 51
>Flu672
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCAAGCTGAACAGACTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGTGGAGCTACAACGCCGAGCT
GCTGGTGGCCCTGGAGAATCAGCACACCATCGACCTGACCGACAGCGAGATGAACAAGCTGTTCATGAAGACCATGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 52
>Flu679
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 53
>Flu680
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 54
>Flu681
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 55
>Flu682
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCGTGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 56
>Flu683
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCGTGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 57
>Flu685
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCATGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGGTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 58
>Flu686
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCATGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 59
>Flu687
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCATGCTGAACCTGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 60
>Flu688
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACATGCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 61
>Flu689
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACTTCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 62
>Flu690
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACTTCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 63
>Flu691
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCATCCTGAACTTCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 64
>Flu692
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACATCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 65
>Flu693
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCCTGCTGAACATCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 66
>Flu695
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCGTGCTGAACATCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGCTGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 67
>Flu696
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCGTGCTGAACATCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATCAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 68
>Flu697
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCGTGCTGAACATCCTGATCGGCAAGACCAACGAGAAGTTCCATCAGATCGAGAAGGAGTTC
AGCGAGGTGGAGGGCAGAGTGCAAGACCTGGAGAAGTACGTGGAGGACACCAAGATCGACCTGGACAGCTACAACGCCGAGCT
GCTGGTGGCCCTGATGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 69
>Flu707
ATGAAGACCATCATCGCCCTGAGCAACATCCTGTGCCTGGTGTTTGCTCAGAAGATCCCCGGCAACGACAACAGCACCGCCAC
CCTGTGCCTCGGCCACCACGCCGTGCCCAACGGCACTATCGTAAAGACGATTACCAACGACAGAATCGAGGTGACCAACGCCA
CCGAGCTGGTGCAGAACAGCAGCATCGGCGAGATCTGCGACAGCCCCCATCAGATCCTGGACGGCGGCAACTGCACCCTGATC
GACGCCCTGCTGGGCGACCCTCAGTGCGACGGCTTTCAGAACAAGGAGTGGGACCTGTTCGTGGAGAGAAGCAGAGCCAACAG
CAACTGCTACCCCTACGATGTGCCGGACTACGCTAGCCTGAGAAGCCTGGTGGCTAGCAGCGGCACCCTGGAGTTCAAGAACG
AGAGCTTCAACTGGACCGGCGTGAAGCAGAACGGCACATCTAGCGCCTGCATCAGAGGCAGCAGCAGCAGCTTCTTCAGCAGA
CTGAACTGGCTGACAAGCCTGAACAACATCTACCCCGCTCAGAACGTGACCATGCCCAACAAGGAGCAGTTCGACAAGCTGTA
CATCTGGGGCGTGCACCACCCCGACACCGACAAGAATCAGATCAGCCTGTTCGCTCAGAGCAGCGGCAGAATCACCGTGAGCA
CCAAGAGATCTCAGCAAGCCGTGATCCCCAACATCGGCAGCAGACCTAGAATCAGAGACATCCCTAGCAGAATCAGCATCTAC
TGGACCATCGTGAAGCCTGGTGACATCCTGCTGATCAACAGCACCGGCAACCTGATCGCCCCTAGAGGCTACTTCAAGATCAG
AAGCGGCAAGAGCAGCATCATGAGAAGCGACGCCCCCATCGGCAAGTGCAAGAGCGAGTGCATCACCCCCAACGGAAGCATCC
CCAACGACAAGCCCTTTCAGAACGTGAACAGAATCACCTACGGCGCCTGCCCTAGATACGTGAAGCAGAGCACCCTGAAGCTG
GCCACCGGCATGAGAAACGTGCCCGAGAAGCAGACAAGAGGCATCTTCGGCGCCATCGCCGGCTTCATCGAGAACGGCTGGGA
GGGCATGGTGGACGGCTGGTACGGCTTCAGACATCAGAACAGCGAGGGCAGAGGCCAAGCCGCCGACCTCAAGAGCACCCAAG
CCGCCATCGATCAGATCAACGGCAAGCTGAACAGACTGATCGGCAAGACCAACGAGAAGTTCCATCAGATCGAGAAGGAGTTC
AGCGAGCCCGAGGGCAGAGTGCAAGACCTGGAGAAGTACGTGGAGGACACCAAGATCGACCTGGACAGCTACAACGCCGAGCT
GCTGGTGGCCCTGGAGAATCAGCACACCATCGACCTGACCGACAGCGAGATGAACAAGCTGTTCGAGAAGACCAAGAAGCAGC
TGAGAGAGAACGCCGAGGACATGGGGAACGGCTGCTTCAAGATCTACCACAAGTGCGACAACGCCTGCATCGGCAGCATCAGA
AACGAGACCTACGACCACAACGTGTACAGAGACGAGGCCCTGAACAACAGATTTCAGATCAAGGGCGTGGGCAGCGAGAACCT
GTACTTCCAAGGCGGCAGCAAGGGCTACATCCCCGAGGCCCCTAGAGACGGCCAAGCCTACGTGAGAAAGGACGGCGAGTGGG
TGCTGCTGAGCACCTTCCTGGGGCATCACCATCACCATCACTGATGA
SEQ ID NO: 70
H1 WT foldon sequence: Signal sequence-HA-TEV cleavage site-foldon-His tag
MKAILVVLLYTFTTANADTLCIGYHANNSTDTVDTVLEKNVTVTHSVNLLEDKHNGKLCKLGGVAPLHLGKCNIA
GWILGNPECESLSTARSWSYIVETSNSDNGTCYPGDFINYEELREQLSSVSSFERFEIFPKTSSWPNHDSNKGVT
AACPHAGAKSFYKNLIWLVKKGNSYPKLNQTYINDKGKEVLVLWGIHHPPTTADQQSLYQNADAYVFVGTSRYSK
KFKPEIATRPKVRDQEGRMNYYWTLVEPGDKITFEATGNLVVPRYAFTMERNAGSGIIISDTPVHDCNTTCQTAE
GAINTSLPFQNVHPVTIGKCPKYVKSTKLRLATGLRNVPSIQSRGLFGAIAGFIEGGWTGMVDGWYGYHHQNEQG
SGYAADLKSTQNAIDKITNKVNSVIEKMNTQFTAVGKEFNHLEKRIENLNKKVDDGELDIWTYNAELLVLLENER
TLDYHDSNVKNLYEKVRNQLKNNAKEIGNGCFEFYHKCDNTCMESVKNGTYDYPKYSEEAKLNREKIDGVGSENL
YFQGGSKGYIPEAPRDGQAYVRKDGEWVLLSTFL [symbol] HHHHHH
SEQ ID NO: 71
>H3 WT foldon sequence: Signal sequence-HA-TEV cleavage site-foldon-His tag
MKTIIALSNILCLVFAQKIPGNDNSTATLCLGHHAVPNGTIVKTITNDRIEVTNATELVQNSSIGEICDSPHQIL
DGGNCTLIDALLGDPQCDGFQNKEWDLFVERSRANSNCYPYDVPDYASLRSLVASSGTLEFKNESFNWTGVKQNG
TSSACIRGSSSSFFSRLNWLTSLNNIYPAQNVTMPNKEQFDKLYIWGVHHPDTDKNQISLFAQSSGRITVSTKRS
QQAVIPNIGSRPRIRDIPSRISIYWTIVKPGDILLINSTGNLIAPRGYFKIRSGKSSIMRSDAPIGKCKSECITP
NGSIPNDKPFQNVNRITYGACPRYVKQSTLKLATGMRNVPEKQTRGIFGAIAGFIENGWEGMVDGWYGFRHQNSE
GRGQAADLKSTQAAIDQINGKLNRLIGKTNEKFHQIEKEFSEVEGRVQDLEKYVEDTKIDLWSYNAELLVALENQ
HTIDLTDSEMNKLFEKTKKQLRENAEDMGNGCFKIYHKCDNACIGSIRNETYDHNVYRDEALNNRFQIKGVGSEN
LYFQGGSKGYIPEAPRDGQAYVRKDGEWVLLSTFLGHHHHHH
SEQ ID NO: 72
HA ectodomain sequence from Bri18 shown in FIG. 2
MKAILVVLLYTFTTANADTLCIGYHANNSTDTVDTVLEKNVTVTHSVNLLEDKHNGKLCKLGGVAPLHLGKCNIAG
WILGNPECESLSTARSWSYIVETSNSDNGTCYPGDFINYEELREQLSSVSSFERFEIFPKTSSWPNHDSNKGVTAACP
HAGAKSFYKNLIWLVKKGNSYPKLNQTYINDKGKEVLVLWGIHHPPTTADQQSLYQNADAYVFVGTSRYSKKFKPE
IATRPKVRDQEGRMNYYWTLVEPGDKITFEATGNLVVPRYAFTMERNAGSGIIISDTPVHDCNTTCQTAEGAINTS
LPFQNVHPVTIGKCPKYVKSTKLRLATGLRNVPSIQSRGLFGAIAGFIEGGWTGMVDGWYGYHHQNEQGSGYAA
DLKSTQNAIDKITNKVNSVIEKMNTQFTAVGKEFNHLEKRIENLNKKVDDGFLDIWTYNAELLVLLENERTLDYHDS
NVKNLYEKVRNQLKNNAKEIGNGCFEFYHKCDNTCMESVKNGTYDYPKYSEEAKLNREKIDGV
SEQ ID NO: 73
HA ectodomain sequence from Darw21 shown in FIG. 2
MKTIIALSNILCLVFAQKIPGNDNSTATLCLGHHAVPNGTIVKTITNDRIEVTNATELVQNSSIGEICDSPHQILDGGNC
TLIDALLGDPQCDGFQNKEWDLFVERSRANSNCYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQISLFAQSSGRITVSTKRSQQAVIPNIGSRPRIR
DIPSRISIYWTIVKPGDILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALENQHTIDLTDSEMNKLFEKTKKQLRENAEDMGN
GCFKIYHKCDNACIGSIRNETYDHNVYRDEALNNRFQIKGV

### EXAMPLES

### Example 1—Design and Construction of Influenza A Strain HA Constructs

### (a) H1 Influenza A Strain (Group 1)

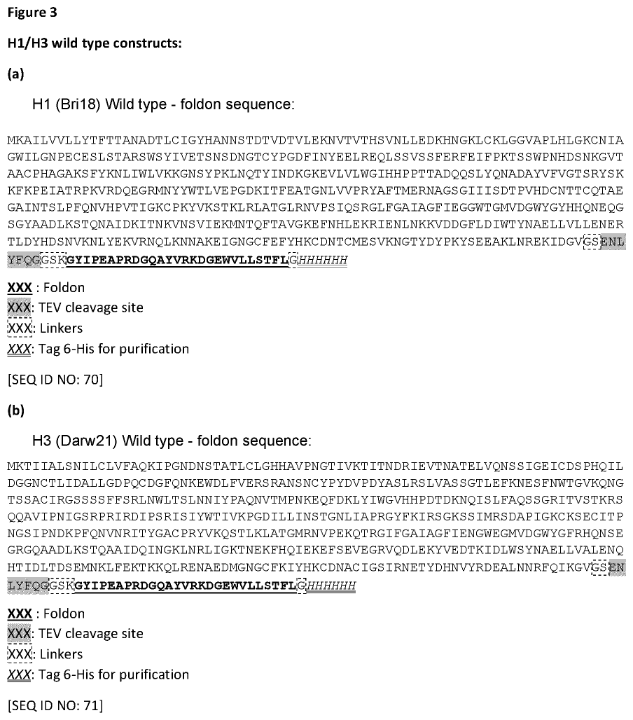

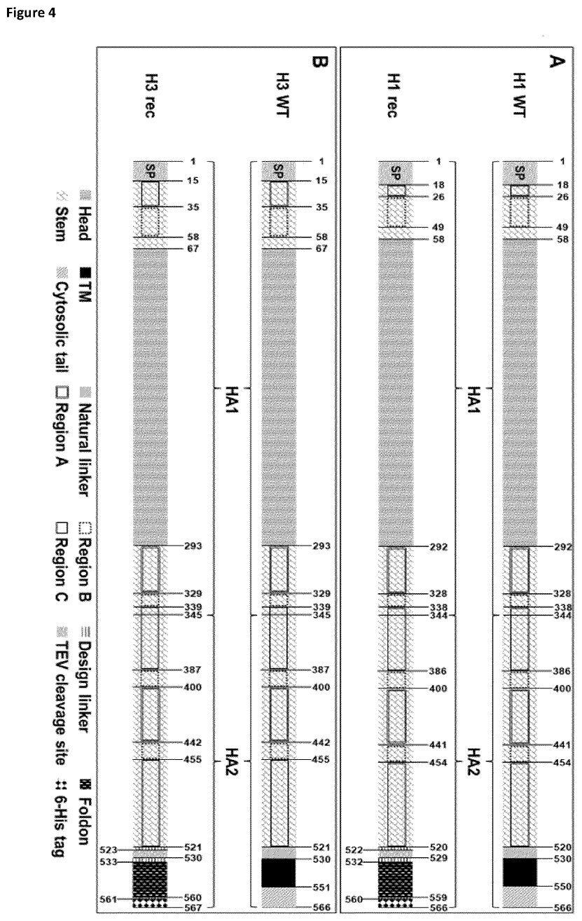

H1 haemagglutinin expressed recombinantly has been shown to be suboptimal, with difficulty maintaining its trimeric conformation. To attempt to mitigate the risk of losing the ectodomain trimeric conformation and to improve both manufacturability and antigenic features, a thorough analysis of the A/Brisbane/02/2018 (H1N1) HA 3D modeled structure was performed using MOE to identify potentially interesting positions in HA2 and HA1 helix B (see FIGS. 1, 2 and 3) and its surrounding environment to introduce mutations to improve trimer conformation stability.

Using the A/Brisbane/02/2018 (H1N1) strain HA 3D modeled structure, several positions were identified to mutate to attempt to stabilize the trimeric conformation: Positions 450, 453, 460, 464, 467 where mutation to hydrophobic residues could promote hydrophobic exlusion and favour trimer formation, with formed trimer coiled coil structure stabilized by these hydrophobic interactions.

Positions such as 322, 436 and 438 where mutation towards polar charged residues could favour protomer interaction.

Positions 395, 447 in HA2 helix A and HA2 helix B, respectively, could be targeted to increase HA1/HA2 interaction either by hydrophobic reinforcement or cavity filling/hydrogen bond introduction.

The mutations were introduced as single amino acid substitutions individually or in combination for a potentially additive effect. Combinations of mutations that were used in ectodomain constructs are shown in Tables 1 and 2.

### (b) H3 Influenza A Strain (Group 2)

A thorough analysis of the A/Darwin/9/2021 H3N2 HA 3D modeled structure was performed using MOE to identify potentially interesting positions in HA2 and HA1 helix B (see FIGS. 2, 3 and 4) and its surrounding environment to introduce mutations to improve trimer conformation stability.

Using the A/Darwin/9/2021 H3N2 strain HA 3D modeled structure, several positions were identified to mutate to attempt to stabilize the trimeric conformation:

Positions 347, 440, 451, 454, 455, 468 where mutation to hydrophobic residues could promote hydrophobic exlusion and favour trimer formation, with formed trimer coiled coil structure stabilized by these hydrophobic interactions.

Positions 418 found at the end of the interloop region connecting helix A and B, for which mutation to proline could prevent post-fusion conformation formation through steric hindrance introduced by the particular side chain from proline.

Positions 369, 399, 448 in HA2 helix A for the 1st and HA2 helix B for the last two, respectively, could be targeted to increase HA1/HA2 interaction either by hydrophobic reinforcement or cavity filling/hydrogen bond introduction.

Positions such as 437 where mutation towards polar and charged residues could favour protomer interaction.

The mutations were introduced as single amino acid substitutions individually or in combination for a potentially additive effect. Combinations of mutations that were used in ectodomain constructs are shown in Table 3.

### Example 2—Cloning, Protein Expression and Purification

### Cloning

Genes were codon optimized for human protein expression, synthesized and cloned into pmaxCloning™ vector (Lonza, Cat. VDC-1040) by GENEWIZ, using EcoRI/Notl restriction sites. The pmaxCloning™ vector backbone contains the immediate early promoter of cytomegalovirus (PCMV IE) for protein expression, a chimeric intron for enhanced gene expression and the pUC origin of replication for propagation in E. coli. The bacterial Promoter (P) provides kanamycin resistance gene expression in E. coli. The multiple cloning site (MCS) is located between the CMV promoter and the SV40 polyadenylation signal (SV40 poly A).

Each construct comprised a sequence encoding an Influenza haemagglutinin (HA) ectodomain of SEQ ID NO: 1 or 2, with mutations including those shown in Table 1, 2 or 3. All constructs were fused at the C-terminus with a TEV cleavage site followed by a foldon, followed by a 6×His-tag (except for mut10 where there was no TEV cleavage site).

### Expression

Expi293F™ cells (ThermoFisher, Cat. A14528) were used for recombinant protein expression. Cell culture and transfection were performed following manufacturer's instructions. Small scale cultures (3 mL cultures in 24-deep well plates) were used for screening of candidates and medium scale cultures (125 mL) were performed on selected top candidates.

The day before transfection, cell density and viability were assessed using a TC20™ Automated Cell Counter (Bio-Rad). Cells were seeded in fresh, prewarmed Expi293™ Expression medium (ThermoFisher, Cat. A1435102) at a density of 2·10⁶ cells/mL and cultured in a humidified 8% CO₂ incubator at 372C and 110 rpm. The day of the transfection, cell density and viability were assessed (viability>95%) and cells were diluted to a final density of 3·10⁶ cells/mL with fresh, prewarmed Expi293™ Expression medium. Transfection was performed using ExpiFectamine™ 293 Transfection Kit (Thermofisher, Cat. A14524), containing transfection enhancers and ExpiFectamine 293 transfection reagent. Briefly, plasmid DNA and transfection reagent were diluted separately in OptiMEM medium (Thermofisher, Cat.31985062) and incubated for 5 min at RT (1 μg of plasmid DNA was used per 1 mL of cell culture). Both mixtures were then combined and incubated for 20 additional min at RT. The ExpiFectamine™ 293/plasmid DNA complexes solution was then carefully added to the cells. Cells were cultured in a humidified 8% CO₂ incubator at 37° C. and 110 rpm. On day 1 post-transfection (18-22 h post-transfection), ExpiFectamine™ 293 Transfection Enhancers 1 and 2 were added. On day 4 post-transfection cells were harvested by centrifugation at 42C and 5000×g for 10 min. Cell pellets were discarded and supernatants were supplemented with Complete™ Protease Inhibitor Cocktail (Roche, Cat. 11697498001). Protein expression was checked by SDS-PAGE and Western blot before purification (data not shown).

### Purification

Purification on HTP expression (2.5 mL culture on a 24 Deep Well format) was performed by adding 200 μL of Nickel Sepharose Excel (GE) slurry preequilibrated in buffer A (20 mM Bicine, 500 mM NaCl, 20 mM Imidazole, pH 8.3) with 0.2 mM 4-(2-aminoethyl) benzenesulfonyl fluoride hydrochloride (AEBSF) (Sigma) and 20 mM Bicine pH 8.3. After rocking at 900 rpm overnight, the samples were transferred to a 96 DW Thompson filter plate and washed 3 times with 1 mL of buffer A under negative pressure. The proteins were eluted by centrifugation (10 minutes at 800 g) with 2 times 110 μL of buffer B (20 mM Bicine, 500 mM NaCl 500 mM Imidazole, pH 8.3), desalted by PD multitrap G-25 and analysed by SDS-PAGE.

Purification on medium scale expression (125 mL culture) was performed by gravity flow column packed with 3 mL of Nickel Sepharose Excel (GE) preequilibrated in buffer A (20 mM Bicine, 500 mM NaCl, 20 mM Imidazole, pH 8.3). After sample loading, the resins were washed with 15 CV of buffer A and the proteins were eluted with 4CV of buffer B (20 mM Bicine, 500 mM NaCl 500 mM Imidazole, pH 8.3). The proteins were then concentrated using Vivaspin 20 with a cut-off of 10 KDa at 3000 g at 4° C. The concentrated samples were loaded onto Superdex 200 increase 10/300 (GE) or Superdex 200 16/600 (GE) equilibrated in buffer C (20 mM bicine, 150 mM NaCl, pH 8.3) with a flow rate of 0.75 ml/min. Fractions corresponding to the proteins of interested were pooled together, filtered 0.22 μM and stored at −80° C.

Protein concentrations were determined by RCDC assay (Biorad) and the purity by SDS-PAGE.

### Characterization

### UPLC—Ultra High Performance Liquid Chromatography

The stability of the trimer assembly of the semi-purified HA constructs from the high throughput screening (HTS) experiments was assessed by HPLC-SEC-UV. Briefly, 10 μl of each preparation was injected on a 4.6×150 mm BEH column with 200A pore size (Acquity) at 0.3 ml/min. UV at 280 nm was recorded during the 10 minutes run. The column was maintained at 30° C. and samples at 8° C. during the experiment. Elution times of HA peaks were compared to calibration standards (Waters BEH200 SEC Protein Standard Mix, ref. Waters 186006518). Based on retention times, peak areas in predefined regions of elution for aggregates, oligomers, trimers and monomers respectively, were recorded.

### BLI—Bio-Layer Interferometry

An Octet Red instrument (Pall-ForteBio, Menlo Park, USA) was used for all the IgG binding measurements. All measurements were made in Kinetics Buffer (KB) (Pall-ForteBio, Menlo Park, USA) 1×. Mutant proteins were prepared by diluting the protein solution in KB 1× to a concentration of 263 nM and immobilised on Ni-NTA sensortips for 180 seconds. Washing of unbound ligand was performed by incubation of sensortips in buffer solution for 60 seconds. Binding was monitored upon immersion into a solution of Fl6v3 or CR9114 (4000 nM-62.5 nM) in KB for 300 seconds. Dissociation was monitored for 60 seconds upon immersion in KB 1× buffer.

### Differential Scanning Fluorimetry

Thermal unfolding of 0.5 mg/ml HA was monitored between 2° and 95° C. by fluorescence of endogenous Trp (at 330 and 350 nm using 290 nm excitation in a NanoDSF NT-Plex instrument (Nanotemper Technologies, Munich, Germany)) or added Sypro Orange (at 640 nm using 498 nm emission in a LightCycler 480, Roche, Basel, Switzerland). Temperature was increased at a rate of 1° C./min and 0.3° C./s, respectively. The fluorescence intensities (SYPRO) or the ratio of the intensities at 330 and 350 nm (Trp) were used to calculate the change per temperature increase and the position of the main transitions was assessed qualitatively.

### Differential Scanning Calorimetry (DSC)

Unfolding was followed at 0.4 mg/ml HA between 2° and 95° C. at 5° C. intervals in a MicroCal PAEQ-n automatedDSC (Malvern Panalytical, France). Data were analyzed in the integrated software to determine the melting temperature.

### AUC

Sedimentation velocity analytical ultracentrifugation (SV-AUC) was performed to determine the molecular weight and stoichiometry of the proteins by measuring the rate at which molecules move through the buffer in response to centrifugal force.

SV-AUC was performed using a Beckman-Coulter Optima AUC analytical ultracentrifuge with an AN-60Ti rotor and the protein was at 0.5 mg/mL and frozen at −80° C. before experiment. The selected rotor speed for the run was 20,000 rpm, the temperature was maintained at 20° C. and the absorbance profile at 280 nm was recorded every min.

Protein-specific density and solvent density were calculated by using the software SEDNTERP 1 (Sedimentation Interpretation Program Version 1.11). The dataset was analysed with Sedfit 15.01b program using a continuous size distribution c(s) model.

### Circular Dichroism Spectroscopy

Far UV CD spectra were taken on a Chirascan spectrometer at a HA candidate concentration of 0.2 mg/ml in 4 mM Bicine pH 8.3 and 30 mM NaCl. Spectra were taken between 190 and 260 nm using a cell with 0.5 mm pathlength and a 1 nm bandwith. Temperature was held at 25° C. Thermal melts up to 95 C were performed by increasing the temperature 1 degree per minute, holding the temperature for 2 min before taking a spectrum after every 5 degree increase.

### Example 3—Results for A/Brisbane/02/2018 (H1N1) Ectodomain Constructs

TABLE 4

| | productivity | | | Octet | | Octet (7D37C | | nanoDSF |
|---|---|---|---|---|---|---|---|---|
| Ectodomain | (mg/ml) | | | nm | | and trypsin) | | Tm1/Tm2 |
| construct | (mg/ml) | UPLC | AUC | FI6V3*** | CR9114*** | FI6V3*** | CR9114*** | (° C.) |
| Mut10 | 17.7 | mo* | 3m | 0.9682 | 0.8046 | 0.7372 | 0.7288 | 58.1/69.7 |
| Mut17 | 17 | mo* | 3m | 0.8781 | 0.8137 | 0.748 | 0.7536 | n.o./69.9 |
| Mut18 | 17.3 | mo* | 3m** | 0.8109 | 0.7306 | 0.4837 | 0.4638 | 54.3/66.3 |
| Mut23 | 60.7 | mo* | 3m | 0.9837 | 0.9014 | 0.7449 | 0.8083 | 62.4 **** |
| Mut24 | 92 | mo* | 3m | 0.9298 | 0.8723 | 0.4056 | 0.0617^{$} | 53.7/68.7 |
| Mut27 | 29.7 | 3m/mo* | agg** | 0.3976 | 0.3366 | 0.3131 | 0.3143 | 59.6/70 |
| control | 16.7 | n.d. | 3m | 0.8558 | 0.8261 | 0.697 | 0.6926 | 56.1/64.9 |

n.d. not determined
n.o. not observed
*aberrant elution volume suspected due to aspecific interaction with column
**polydisperse
***KD similar to control in all cases, except 100-fold reduced affinity Mut24^{$}
**** convoluted peaks
3m—trimer
mo—monomer

A first batch of constructs based on Bri18 HA was designed, prepared and assessed according to the previous Examples. In all, 33 ectodomain constructs were investigated (see Table 2). For 6 lead constructs (see Table 1), read-outs from characterisation studies described in Example 2 are shown here. The read-outs were combined to produce an aggregated view of the data in Table 4. AUC and DSF were carried out after TEV cleavage. Unless otherwise noted, experiments were carried out with trimerisation domain present.

All constructs except Mut27 maintained the trimeric form as confirmed by AUC. The aberrant elution by UPLC is attributed to aspecific interaction with the matrix of the BEH-column. Consistent with this, Mut27 showed a reduced response to antibody recognition. Mut18 and Mut24 also showed poor antibody recognition. Mut10, Mut17 and Mut23 maintained antibody recognition and increased trimer stability. Three unfolding events were observed upon HA unfolding by nanoDSF.

The highest melting temperature (at ca. 80° C.) was attributed to the dissociation of the foldon by comparison with foldon only stability (not shown). Tm1 in Table 4 is suggested to represent unfolding of the HA head based on its disappearance by acidification, while Tm2 is suggested to represent unfolding of the stem. Both Tm1 and Tm2 increase by at least 2° C. in the three mutants, indicating an overall stabilization of the HA trimer due to the mutations. When the foldon was cleaved from the control and Mut10, Mut17 and Mut23, the mutants showed increased trimeric populations as determined by AUC and increased thermal stability as determined by nanoDSF compared to the control (not shown).

### Example 4—Results for A/Darwin/9/2021 H3N2 Ectodomain Constructs

### High-Throughput Screening was Carried Out for the Darw21 Constructs:

Constructs were characterised with the following read-outs:
Productivity: The quantity of protein (expressed in mg/L) after purification was measured by colorimetric method. Previous analysis suggested that higher productivity is often associated with a more stable folding. We used this parameter as a selection criterion for full ectodomain constructs.
UPLC-SEC: to discriminate between the soluble forms of the proteins (either monomer, trimer, higher order oligomers or soluble aggregates). Each form displays as a (partly) separated peak on the elution profile.
BLI: biosensor technology used to quantify the binding of structure-specific immunotools to the immobilised mutants. Here, CR9114 and Fl3v6 monoclonal antibodies were used to probe the stem region of the proteins in comparison to the control. The binding was recorded as an increase of the thickness (expressed in nm) of the sensing surface as the antibody ligand binds to the immobilised HA protein mutant. In previous experiments, we observed an increase of the binding to more stable HA trimers.
Differential scanning fluorimetry: protein folding fingerprinting technology used to confirm the stability of the protein folding by measuring the unfolding temperature (Tm, expressed in ° C.) compared to that of the reference Darw21 sequence. This read-out is used to ensure that the mutation pattern does not disrupt the protein folding. Read-outs were based on intrinsic protein fluorescence or by following changes in fluorescence of the externally added hydrophobic SYPRO Orange probe.
Differential scanning calorimetry: protein folding fingerprinting technology used to confirm the stability of the protein fold by measuring the unfolding temperature (Tm, expressed in ° C.) compared to that of the reference Darw21 sequence. Heat-capacity read-out is used to ensure that the mutation pattern does not disrupt the protein folding.
Circular dichroism spectroscopy: protein folding fingerprinting technology used to confirm the structure and the stability of the protein folding by measuring the secondary structure composition and its denaturation compared to that of the reference Darw21 sequence. The read-out is used to ensure that the mutation pattern does not alter the protein folding.

135 constructs were analyzed for their expression, purification, oligomeric state (by UPLC) and antigenic features by recognition of CR9114 and Fl3v6 antibodies. Twenty-six constructs that produced HA trimers with robust antibody binding capacity at levels of or levels above the Darwin2l control or with apparent increased affinity were selected for in depth characterization.

After selection of 26 candidates, midscale expression and purification were performed both to confirm the productivity of each selected candidate and to provide more protein material for characterization purposes. At midscale, the following characterization assays were performed:
UPLC for assessment of the trimer conformation after cleavage of the foldon. This read-out offers analysis of the intrinsic oligomerization state of the HA trimer.
nanoDSF (measuring the change in intrinsic fluorescence) and DSF (measuring the change in fluorescence of the exogenous added SYPRO Orange) were used to assess stability of the trimer after cleavage of the foldon to assess the intrinsic stability of the HA trimers. DSC (measuring the change in heat capacity) and CD spectroscopy (measuring the change in secondary structure) were performed on top constructs in addition to the prior experiments to help assigning unfolding to specific domains of the HA trimer.
BLI was repeated on foldon-containing samples using CR9114 and Fl6v3 as a probe for the conformation of the stem region of the constructs, relative to the reference.

Results are shown in Table 5. Overall, nanoDSF and DSF measurements showed main transitions at equivalent melting temperatures. Five candidates increased the melting temperature 2 to 5° C. compared to the control: Flu632, Flu639, Flu643, Flu691 and Flu689. All of these, except Flu639 and Flu643, increased affinity for Fl6v3 approximately 10-fold or higher. Several other candidates increased Fl6v3 affinity 5-fold or less without increasing the melting temperature.

The stability and antibody affinity of the 5 top candidates were analyzed further. In addition, Flu690, that had the highest increase in Fl6v3 affinity (appr. 100 fold), increased CR9114 affinity (by ten-fold) but was not thermostabilized, and Flu687 that increased affinity for Fl6v3 5-fold but did not increase CR9114 affinity nor the melting temperature were analyzed.

Unfolding of the H3 Darwin 21 controls showed a single transition when followed by Trp fluorescence. In contrast, two transitions were observed when unfolding was followed by SyproOrange that binds to the exposed hydrophobic residues and in differential scanning calorimetry. Thermal unfolding followed by circular dichroism spectroscopy also indicated two transitions due to two-step unfolding of the helical content. However, R-sheet structure appeared to unfold in one cooperative transition.

Flu632 and Flu691 showed similar profiles to the control in fluorescence and calorimetry but with melting temperatures of each transition shifted to higher temperatures by 5° C., suggesting a stabilized trimerization helix. Two transitions in both helical and sheet content were observed, indicating strong cooperativity in the unfolding of the entire structure, but possibly hints towards heterogeneous populations. The same characteristics were observed for Flu689 except that the mutations only led to an increase of the melting transitions by 2° C. and this was the only stabilized construct that also appeared to increase CR9114 affinity, and this by approximately 10-fold.

Differential scanning fluorimetry measured by Trp or SyproOrange and thermal unfolding followed by circular dichroism spectroscopy indicated that Flu639 and Flu643 increased stability of the helical and sheet segments in the structure and increased cooperativity of unfolding so that only one transition was observed at melting temperature 5° C. above the control. These data are consistent with a homogeneous stabilized HA trimer, but the constructs did not show higher affinity for the antibodies than the control.

TABLE 5

| | | | | | | | | | | α-helical |
|---|---|---|---|---|---|---|---|---|---|---|
| | | | | | | | | | | unfolds |
| Ectodomain | productivity | | nanoDSF | DSF | Octet (nm) | | Octet (trypsin) | | calorimetry | before |
| construct | (mg/L) | UPLC | Tm (C.) | Tm (C.) | FI6V3 | CR9114 | FI6V3 | CR9114 | Tm (C.) | β-sheet** |
| Flu622 | 41.16992 | 3m | 61.4 | 60.54 | 0.2752 | | | | | |
| Flu629 | 37.8288 | 3m | 60.5 | 60.29 | 0.3115 | | | | | |
| Flu632* | 61.9916 | 3m | 65.6 | 65.99 | 0.4107 | 0.38 | 0.3788 | 0.3111 | 63.2 | yes |
| Flu638 | 50.85152 | 3m | 62.0 | 61.94 | 0.4486 | | | | | |
| Flu639* | 83.50656 | 3m | 63.2 | 63.18 | 0.2446 | 0.31 | 0.3538 | 0.2339 | 65.04 | no |
| Flu643* | 21.0256 | 3m | 64.8 | 64.58 | 0.2971 | 0.13 | 0.3991 | 0.2049 | 66.8 | no |
| Flu650 | 14.792 | 3m | 59.6 | 59.72 | 0.1109 | | | | | |
| Flu672 | 10.64024 | 3m | 58.1 | 58.23 | 0.2222 | | | | | |
| Flu681 | 34.50048 | 3m | 59.8 | 59.96 | 0.4895 | | | | | |
| Flu688 | 27.9072 | 3m | 59.9 | 60.13 | 0.3457 | | | | | |
| Flu691 | 39.05792 | 3m | 64.4 | 64.58 | 0.5042 | 0.51 | 0.6174 | 0.4714 | 60.1 | yes |
| Flu697 | 28.5252 | 3m | 60.7 | 60.71 | 0.475 | | | | | |
| Flu707 | 52.02 | 3m | 59.8 | 59.88 | 0.3135 | | | | | |
| Flu752 | 15.31104 | 3m | No | No | 0.1895 | | | | | |
| | | | trans. | Trans. | | | | | | |
| Flu679 | 68.8 | 3m | 61.9 | 60.54 | 0.4628 | | | | | |
| Flu680 | 76.8 | 3m | 58.5 | 58.48 | 0.509 | | | | | |
| Flu682 | 77.6 | 3m | 62.0 | 61.94 | 0.3755 | | | | | |
| Flu683 | 56 | 3m | 59.7 | 60.05 | 0.4219 | | | | | |
| Flu685 | 58.4 | 3m | 59.4 | 59.8 | 0.5698 | | | | | |
| Flu686 | 62.4 | 3m | 62.0 | 61.94 | 0.4356 | | | | | |
| Flu687* | 64 | 3m | 59.5 | 59.88 | 0.3001 | 0.28 | 0.6167 | 0.5078 | 62.65 | |
| Flu689* | 61.6 | 3m | 62.5 | 62.69 | 0.4055 | 0.48 | 0.7097 | 0.5447 | 65.13 | yes |
| Flu690* | 52.8 | 3m | 59.8 | 48.42 | 0.3915 | 0.46 | 0.6288 | 0.4847 | 62.92 | yes |
| Flu692 | 59.2 | 3m | 60.7 | 58.15 | 0.3234 | | | | | |
| Flu693 | 53.6 | 3m | 57.8 | 60.62 | 0.4195 | | | | | |
| control | 34.0838 | 3m | 59.9 | 60.87 | 0.36 | 0.3595 | 0.4783 | 0.3021 | 60.6 | yes |

*Flu constructs with at least 10-fold improvement of KD for FI6v3 were selected for further characterization
**unfolding pattern based on integrated data from calorimetry, nanoDSF, DSF and secondary structure by circular dichroism spectroscopy

### Example 5—Transfection and Purification of Mut10 Hemagglutinin Ectodomain and Fab Fl6v3

Plasmids encoding either the Mut10 Hemagglutinin Ectodomain (HA) or Fl6v3 Fab domain, each engineered with a C-terminal hexahistidine tag, were transiently expressed in 500 mL of Expi293F™ GnTI-cells (ThermoFisher Scientific). The culture supernatant containing Mut10 HA was harvested 5 days post-transfection and filtered using a 0.22 μm filter prior to purification via nickel affinity chromatography on an AKTA Avant system. The culture supernatant was loaded through a HisTrap column (Cytiva Life Sciences) that was previously equilibrated in equilibration buffer composed of 25 mM HEPES pH 7.5, 150 mM NaCl. Captured protein was eluted by elution buffer composed of 150 mM NaCl, 500 mM imidazole in 25 mM HEPES buffer, pH 7.5, in a step-gradient manner. The step-gradient was performed as follows: starting from 4% elution buffer for 5 column volumes, followed by a gradient from 4-25% elution buffer for 5 column volumes, 25% step elution buffer for 5 column volumes, a 25%-50% gradient for 5 column volumes, 50% elution buffer for 5 column volumes, a 50%-100% gradient of elution buffer for 5 column volumes, then lastly 100% step elution for 5 column volumes. Protein was eluted at 4% and 18% of elution buffer, which corresponds to 20 mM and 90 mM imidazole, respectively. Fractions that eluted at 4% and 18% imidazole were collected and concentrated down to 1 mL using a 10 kDa Amicon Ultra Centrifugal Concentrator filter unit (EMD Millipore), before being filtered on a 0.22 μm filter and loaded onto a HiLoad 16/600 Superdex 200 μg column (Cytiva Life Sciences) pre-equilibrated with equilibration buffer at 1 ml/min. Chromatogram showed two peaks where the first peak eluted at 40-50 mL and a second larger peak eluted between 55-68 mL. Peak fractions were analyzed by SDS-PAGE to assess purity and to identify the target protein, which was shown to have eluted out between 55-69 mL of the second peak and was determined to be >95% pure. Fractions were collected and stored in equilibration buffer at −80° C. until further use.

For Fl6v3 Fab, the culture supernatant was harvested 5 days post-transfection and filtered using a 0.22 μm filter prior to purification via nickel affinity chromatography on an AKTA Avant system. Culture supernatants were loaded onto a HisTrap column (Cytiva Life Sciences) that was equilibrated in equilibration buffer composed of 25 mM HEPES pH 7.5, 150 mM NaCl. Captured protein was eluted by elution buffer composed of 25 mM HEPES pH 7.5, 150 mM NaCl, 500 mM imidazole in a step-gradient manner. The step-gradient was performed in a similar manner as above with slight modification. The step-gradient was performed as follows: starting from 4% step elution buffer for 5 column volumes, followed by a gradient from 4-25% elution buffer for 5 column volumes, 25% step elution buffer for 5 column volumes, a 25%-100% gradient for 5 column volumes, then lastly 100% step elution buffer for 5 column volumes. Protein was eluted at 4% and 6%-22% of elution buffer, which corresponds to 20 mM and 30-110 mM imidazole, respectively. Fractions that eluted out at 4% and 6%-22% imidazole were collected and concentrated to 1 mL using a 10 kDa Amicon Ultra Centrifugal Concentrator filter unit (EMD Millipore), before being filtered on a 0.22 μm filter and loaded onto a HiLoad 16/600 Superdex 200 μg column (Cytiva Life Sciences) pre-equilibrated with equilibration buffer composed of 25 mM HEPES pH 7.5, 150 mM NaCl at 1 ml/min. Chromatogram showed two peaks where the first peak eluted at 46-70 mL and another larger peak eluted between 91-114 mL. Peak fractions were analyzed by SDS-PAGE to assess purity and to identify the target protein, which was shown to have eluted between 91-114 mL of the second peak and was determined to be >95% pure. Fractions were collected and stored in equilibration buffer at −80° C. until further use.

### Crystallization of Mut10:Fl6v3 Complex

Both proteins were concentrated to 10 mg/ml using a 10 kDa Amicon Ultra Centrifugal Concentrator filter unit (EMD Millipore). Mut10 and Fl6v3 Fab were mixed in a 1:1.2 molar ratio, respectively and incubated overnight at 4° C. to ensure complex formation. High throughput crystal screening was performed in 96, 2-well sitting-drop plates, (Art Robbins Instruments) at a protein-to-buffer ratio of 1:1 using the Gryphon Robotics Instrument (Art Robbins Instruments). Crystal screens were incubated at 20° C. in a Formulatrix Rock Imager 1000 (Formulatrix), and drops were imaged on a Fibonacci schedule, (i.e. 0, 1, 2, 3, 5 days etc.). Several crystal hits were obtained, and condition hits are listed in Table 6. Crystals were harvested, cryo-protected with 20% ethylene glycol, flash frozen in liquid nitrogen, and shipped to the Advanced Photon Source at Argonne National Labs for data collection. The best diffracting crystal was found to come from a condition containing 10% w/v 2-propanol, 0.1 M HEPES pH 7.5 and 20% w/v PEG 4000 (the crystal appeared after 8 days and grew to full size by day 13). Diffraction data was processed using HKL2000 to a resolution of 2.9 Angstroms in space group 1213.

### Molecular Replacement and Refinement

The structure of the H1 A/Michigan/45/2015 Ectodomain of Influenza A virus (PDB ID 7KNA), and Fl6v3 Antibody Heavy and Light Chain (PDB ID 3ZTJ), were modified using PHENIX Sculptor and used as search models for molecular replacement in PHENIX Phaser for the Mut10 HA Ectodomain and Fl6v3 Fab, respectively [1-3]. One molecule of the Mut10:Fl6v3 Fab complex was found to occupy the asymmetric unit, and applying symmetry recapitulated the expected HA trimer bound by three copies of Fl6v3 Fab (FIG. 1). PHENIX Refinement and Coot were used to refine the model through iterative rounds of refinement [4-6]. Final validation was performed by MOLPROBITY and statistics are shown in Table 6 [7].

### Structure Analysis and Comparison of Mut10:Fl6v3 Complex

The structure of Mut10 contains a single K395M mutation that is buried between two alpha helices of residues 382-402 and 419-470 of the HA2 polypeptide chain, resulting in a substitution of a positively charged Lysine residue to an uncharged, nonpolar Methionine. Despite the mutation, the structure shows binding of Fl6v3 remains conserved at the epitope. Superposition of a single HA protomer from Mut10 with a single protomer from H1 HA (PDB ID 3ZTN), yields an RMSD of 0.52 across 399 Cα atoms, confirming near identical structural conservation between the two structures.

TABLE 6
Crystallization condition hits for Mut10:F16v3 Complex.

| | | Condition Components | | |
|---|---|---|---|---|
| Screen | Well | Component 1 | Component 2 | Component 3 |
| ProPlex | B3 | 0.1M Na3Citrate pH 5.5 | 0.2M NaAcetate | 10% w/v Peg 4000 |
| ProPlex | B4 | 0.2M NaCl | 10% w/v PEG 4000 | 0.1M MES pH 6.5 |
| ProPlex | B11 | 0.1M Sodium HEPES 7.0 | 15% w/v PEG 4000 | |
| ProPlex | D2 | 0.1M MES 6.5 | 10% w/v PEG 5000 MME | 12% v/v 1-Propanol |
| ProPlex | E4 | 0.1M HEPES pH 7 | 8% PEG 8000 | |
| ProPlex | E5 | 0.1M Tris pH 8 | 8% PEG 800 | |
| ProPlex | E8 | 12% PEG 8000 | 0.1M MgAcetate | 0.1 MOPS pH 7.5 |
| ProPlex | E9 | 0.2M NaCl | 0.1M HEPES pH 7.5 | 12% PEG 8000 |
| ProPlex | F3 | 0.1M MgAcetate | 0.1M MES pH 6.5 | 10% PG 10,000 |
| ProPlex | F5 | | 0.1M Tris pH 8.0 | 8% w/v Peg 20,000 |
| GRAS2 | A2 | 0.2M Ammonium | 20% w/v Polyethylene | |
| | | acetate pH 7.2 | glycol monomethyl | |
| | | | ether 2,000 | |
| | B5 | 0.2M Ammonium | 20% w/v PEG 1,000 | |
| | | formate pH 6.6 | | |
| | F9 | 0.1M Sodium phosphate | 20% w/v PEG 1,000 | |
| | | dibasic dihydrate pH 9.2 | | |
| SG1 | A6 | 0.1M Sodium HEPES pH 7.5 | 20% w/v PEG 4000 | 10% w/v 2-Propanol |

TABLE 7
Data collection and refinement statistics.

| | Mut10:FI6v3 | |
|---|---|---|
| Wavelength | | |
| Resolution range* | 32.78-2.901 | (3.004-2.901) |
| Space group | I 21 3 | |
| Unit cell | 202.043 202.043 202.043 90 90 90 | |
| Total reflections | | |
| Unique reflections* | 29420 | (2773) |
| Multiplicity | | |
| Completeness (%)* | 96.48 | (91.85) |
| Mean I/sigma(I) | | |
| Wilson B-factor | 56.03 | |
| R-merge | 0.484 | (2.098) |
| R-meas | 0.331 | (2.039) |
| R-pim | 0.069 | (0.422) |
| CC1/2 | 0.952 | (0.617) |
| Reflections used in refinement* | 29416 | (2773) |
| Reflections used for R-free* | 1999 | (186) |
| R-work* | 0.2165 | (0.2914) |
| R-free* | 0.2608 | (0.3512) |

| | |
|---|---|
| Number of non-hydrogen atoms | 7782 |
| macromolecules | 7565 |
| ligands | 217 |
| solvent | 0 |
| Protein residues | 968 |
| RMS(bonds) | 0.010 |
| RMS(angles) | 1.22 |
| Ramachandran favored (%) | 96.05 |
| Ramachandran allowed (%) | 3.43 |
| Ramachandran outliers (%) | 0.52 |
| Rotamer outliers (%) | 0.48 |
| Clashscore | 15.89 |
| Average B-factor | 60.48 |
| macromolecules | 59.54 |
| ligands | 92.98 |

*Statistics for the highest-resolution shell are shown in parentheses.

### REFERENCES FOR EXAMPLE 5

1. Bunkóczi, G. and R. J. Read. “Improvement of molecular-replacement models with sculptor.” Acta Crystallogr D Biol Crystallogr 67 (2011): 303-12.
2. Adams, P. D., P. V. Afonine, G. Bunkóczi, V. B. Chen, I. W. Davis, N. Echols, J. J. Headd, L. W. Hung, G. J. Kapral, R. W. Grosse-Kunstleve, et al. “Phenix: A comprehensive python-based system for macromolecular structure solution.” Acta Crystallogr D Biol Crystallogr 66 (2010): 213-21.
3. Liebschner, D., P. V. Afonine, M. L. Baker, G. Bunkóczi, V. B. Chen, T. I. Croll, B. Hintze, L. W. Hung, S. Jain, A. J. McCoy, et al. “Macromolecular structure determination using x-rays, neutrons and electrons: Recent developments in phenix.” Acta Crystallogr D Struct Biol 75 (2019): 861-77.
4. Afonine, P. V., B. K. Poon, R. J. Read, O. V. Sobolev, T. C. Terwilliger, A. Urzhumtsev and P. D. Adams. “Real-space refinement in phenix for cryo-em and crystallography.” Acta Crystallogr D Struct Biol 74 (2018): 531-44.
5. Emsley, P., B. Lohkamp, W. G. Scott and K. Cowtan. “Features and development of coot.” Acta Crystallogr D Biol Crystallogr 66 (2010): 486-501.
6. Emsley, P. and M. Crispin. “Structural analysis of glycoproteins: Building n-linked glycans with coot.” Acta Crystallogr D Struct Biol 74 (2018): 256-63.
7. Williams, C. J., J. J. Headd, N. W. Moriarty, M. G. Prisant, L. L. Videau, L. N. Deis, V. Verma, D. A. Keedy, B. J. Hintze, V. B. Chen, et al. “Molprobity: More and better reference data for improved all-atom structure validation.” Protein Sci 27 (2018): 293-315.

### Example 6—Mouse Immunogenicity Studies

### Study A

The immunogenicity of influenza HA full ectodomain trimeric proteins, containing foldon and based on A/Brisbane/02/2018 H1 sequence was evaluated in naïve CB6F1 mice. Female CB6F1 mice were immunized twice, 28 days apart, and intramuscularly with:
(a) Mut10, 0.2 μg/dose, adjuvanted with AS03A, −1/10 human dose (HD); a total of 20 animals were used in this group (divided into 2 independent studies)
(b) Mut23, 0.2 μg/dose, adjuvanted with AS03A, −1/10 human dose (HD); a total of 20 animals were used in this group (divided into 2 independent studies)
(c) QIV 2019/2020 (commercially available quadrivalent influenza vaccine from GlaxoSmithKline (GSK), containing inactivated split influenza (Flu) virions of the strains A/Brisbane/02/2018 H1N1, A/Kansas/14/2017 H3N2, B/Colorado/06/2017 (B/Victoria) and B/Phuket/3073/2013 (B/Yamagata)), 2.66 μg/strain/dose, not adjuvanted; a total of 8 animals were used in this group (divided into 2 independent studies)
(d) QIV 2019/2020, 0.27 μg/strain/dose, adjuvanted with AS03A, −1/10 human dose (HD); a total of 8 animals were used in this group (divided into 2 independent studies)
(e) NaCl as the placebo control group; a total of 4 animals were used in this group (divided into 2 independent studies)

Due to volume limitation, some assays were performed in pooled sera samples instead of individual serum samples as highlighted on each graph.

Spleens and serum samples were collected at day 42 (corresponding to 14 days post second immunization) and analyzed as described in Example 8 to Example 13 using the assay protocols described in Example 7.

### Study B

Another study was conducted to assess immunogenicity of the influenza HA full ectodomain constructs in primed CB6F1 mice. Female CB6F1 mice were primed at Day 0 intranasally with whole inactivated Influenza virus A/California/7/2009 H1N1, and immunized at Day 28 and Day 56 intramuscularly with:
(a) Mut10, 0.2 μg/dose, adjuvanted with AS03A, −1/10 human dose (HD); a total of 20 animals were used in this group
(b) Mut23, 0.2 μg/dose, adjuvanted with AS03A, −1/10 human dose (HD); a total of 20 animals were used in this group
(c) QIV 2019/2020 (commercially available quadrivalent influenza vaccine from GSK, containing inactivated split influenza virions of the strains A/Brisbane/02/2018 H1N1, A/Kansas/14/2017 H3N2, B/Colorado/06/2017 (B/Victoria) and B/Phuket/3073/2013 (B/Yamagata)), 2.66 μg/strain/dose, not adjuvanted; a total of 8 animals were used in this group
(d) QIV 2019/2020, 0.27 μg/strain/dose, adjuvanted with AS03A, −1/10 human dose (HD); a total of 8 animals were used in this group
(e) PBS as a primed but not immunized control group; a total of 4 animals were used in this group
(f) NaCl as the placebo control group (not primed); a total of 4 animals were used in this group

Spleens and serum samples were collected at day 70 (corresponding to 14 days post second immunization) and analyzed as described in Example 8 to 13 using the assay protocols described in Example 7.

### Example 7—Assay Protocols

### IgG Serology ELISA

Quantification of mouse IgG antibodies was performed by ELISA using whole or split Flu virus or recombinant HA (stem-only proteins) as coating antigens diluted in PBS to reach a concentration of 0.5, 1 or 4 μg/ml, depending on the antigen tested (50 μl/well), and adsorbed overnight at 4° C. in 96-well microtiter plates (Maxisorb Immunoplate Nunc 439454). The plates were then incubated for 1 hour at 37° C. with 100 μl/well of PBS+10% milk (saturation buffer). Twelve two-fold dilutions of sera (diluted in PBS+1% BSA+0.1% Tween 20, further referred to as dilution buffer) were added to the coated plates (50 al/well), and incubated for 90 minutes at 37° C. The plates were then washed four times with PBS+0.1% Tween 20. Peroxydase-conjugated goat anti-mouse IgG (Jackson 115-035-003) diluted 1/250 in dilution buffer was added to each well (50 al/well), and incubated for 1 hour at 37° C. After another washing step, plates were incubated 20 minutes at RT with OPDA substrate (Sigma P4664). The reaction was stopped with H2SO4 2N, and optical densities were read at 490-620 nm. The titers were expressed as ELISA 50% endpoint titers corresponding to the dilution of sample corresponding to an optical density of 1.5 (50% of the high plateau). In the absence of detection of binding activity, the corresponding sample was assigned an arbitrary titer corresponding to half the first serum dilution (1:100), namely 50.

### Hemagglutination Inhibition Assay (HI)

The principle of the HAI assay is based on the ability of specific anti-influenza antibodies to inhibit hemagglutination of red blood cells (RBC) by influenza virus hemagglutinin (HA). Sera were first treated, to remove non-specific inhibitors, with Receptor Destroying Enzyme (Sigma cat. C-8772) at a concentration of 2% (incubation 18H at 37° C.), heat-inactivated 30 min at 56° C. and treated with chicken RBC at a concentration of 5% (incubation 1 h at +4° C.). After pre-treatment, two-fold dilutions of decanted sera were incubated 30 min at RT with 4 hemagglutination units of whole influenza virus. Chicken RBC were then added at a concentration of 0.5% and the inhibition of hemagglutination was scored. The titers were expressed as the reciprocal of the highest dilution of serum that fully inhibited hemagglutination. As the first dilution of sera was 1:20, a titer of 10 was used for samples below the limit of detection.

### FACS-Based In Vitro Influenza Neutralization Assay

MDCK cells were seeded in 96-well cell culture plates (Nunc cat. 167008) the day before the test, with a quantity of 30000 cells/well. Before the testing, sera were pre-treated, to remove non-specific inhibitors, with Receptor Destroying Enzyme (Sigma cat. C-8772) at a concentration of 2% (incubation 18H at 37° C.) and heat-inactivated 30 min at 56° C. Neutralization assay was performed using the following infectivity medium: Ultra-MDCK medium (BioWhittaker cat. BE12-749Q) supplemented with 1% Penicillin-Streptomycin (Invitrogen cat. 15140-122) and 2 μg/ml TPCK-treated trypsin (Sigma cat. T1426). Four three-fold serial dilutions of sera were prepared in monoplicate in 96-well plates. Sera dilutions were mixed with an equal volume of influenza viruses diluted in infectivity medium to reach a MOI of 0.2, corresponding to 6000 TCID50/well for H1N1 strains or a MOI of 0.033, corresponding to approximately 1000 TCID50/well for H5N1 strain. Plates were incubated 2H at 35° C. Six wells were used as the virus only control, and two wells as the cells only control. After incubation, medium was removed from cell containing plates and sera-virus mixes were transferred to these plates. After a centrifugation step (1 h at 2000 rpm), the content of the plates was removed, and replaced by 200 μl of fresh medium. The plates were incubated at 35° C., 5% CO₂ for 16 h. After incubation, the presence of the virus was detected using the following fluorescent staining procedure. Cells were washed with PBS and treated with trypsin to allow detachment. The action of trypsin was blocked by addition of PBS+1% FBS. The cells were harvested in v-bottom plates to perform the staining. After a washing step with PBS+1% FBS, the cells were fixed 20 min at 4° C. with Cytofix/cytoperm reagent (BD cat. 51-2090KZ). After a washing step with Permwash buffer (BD cat. 51-2091KZ), the infected cells were stained 30 min at 4° C. with a FITC-labelled anti-Flu A Nucleoprotein monoclonal antibody (Thermofisher cat. MA1-7322). After a washing step with Permwash buffer, the cells were resuspended with PBS and the plates were analyzed by flow cytometry using a BD Fortessa Flow cytometer and the FlowJo software. The percentage of neutralization was determined for each well, based on the virus only control, considered as 0% of neutralization. The 50% neutralization titers were calculated for each sample using a linear regression method. As the lowest dilution of sera was 1:50, a titer of 17 was used for samples below the limit of detection.

### Antibody Dependent Cell Cytotoxicity (ADCC) Reporter Bioassay (Promega)

For determination of ADCC functionality, the mouse FcgRIII kit from Promega was used, with the following protocol. Serial dilutions of sera were prepared in 96-well plates. Target cells (Expi293 cells transfected in house to express hemagglutinin stem antigen from A/Michigan/45/2015 H1N1 strain) were added to each well (24000 cells/well). Effector cells (Jurkat cells, from the kit, transfected with an enzymatic pathway inducing bioluminescence when activated by antigen-antibody-FcgRIII complex) were also added to each well (60000 cells/well), and incubated 6 hours at 37° C. Luciferase activity was then measured, after having applied the Bio-Glow substrate (provided in the kit), using a Luminescence plate reader. Results were expressed as Area Under the Curve (AUC).

### Intracellular Cytokine Staining (ICS)

The evaluation of Flu-specific T cell responses by ICS was performed using spleen cells collected 14 days post second immunization. Splenocytes were restimulated in vitro (6 hours) with a pool of 15-mers covering the Flu H1-stem sequence (based on A/Michigan/45/2015 H1N1 sequence). Splenocytes T lymphocytes isolation: Spleens were collected and placed in Roswell Park Memorial Institute 1640 medium supplemented with Glutamine, Penicillin/streptomycin, Sodium Pyruvate, non-essential amino-acids and 2-mercaptoethanol). Cell suspensions were prepared from each spleen using a tissue grinder. The splenic cell suspensions were filtered twice (Cell strainer 100 μm). The filter was rinsed with 35 mL (for the first wash) or 12 mL (for the second wash) of PBS EDTA 2 mM. After centrifugation (335 g, 10 min at RT), cells were resuspended in Complete Medium (Roswell Park Memorial Institute 1640 medium supplemented with Glutamine, Penicillin/streptomycin, Sodium Pyruvate, non-essential amino-acids and 2-mercaptoethanol, and 5% Heat inactivated Fetal Calf Serum, further referred to as completed medium. In vitro stimulation: Fresh splenocytes were plated in round bottom 96-well plates at approximately 1 million cells per well. Cells were then stimulated for 6 hours (37° C., 5% C02) with anti-CD28 (clone 37.51) and anti-CD49d (clone 9C10 (MFR4.B)) at 1 μg/mL, with or without 1 μg/mL of 15 mers overlapping peptide pool covering the Flu H1-stem sequence (based on A/Michigan/45/2015 H1N1 sequence). After 2 hours-stimulation at 37° C., Brefeldin A diluted 1/1000 in complete medium was added for 4 additional hours at 37° C. Plates were then transferred at 4° C., overnight. Splenocytes staining: Cells were stained and analyzed using a 6-colour ICS assay. Cells were transferred to V-bottom 96-well plates, centrifuged at 189 g for 5 mir at 4° C. After a washing step with 250 μl of PBS 1% fetal calf serum, cells were resuspended in 50 μl Flow Buffer (PBS 1×, 1% fetal calf serum) containing anti-CD16/32 (clone 2.4G2) diluted 1/50 for 10 min at 4° C. Then, 50 μl Flow Buffer containing anti-CD4-V450 (clone RM4-5) diluted 1/200 and anti-CD8-PerCp-Cy5.5 (clone 53-6.7) antibodies (diluted 1/100) and Live/dead-PO (1/1000) was added for 30 min at 4° C. Cells were centrifuged (189 g for 5 min at 4° C.) and washed with 200 μl Flow buffer. Splenocytes were fixed and permeabilized by adding 200 μl of Cytofix/Cytoperm solution for 20 min at 4° C. Cells were centrifuged (500 g for 5 min at 4° C.) and washed with 200 μl Perm/Wash buffer. After an additional centrifugation step (500 g for 5 min at 4° C.), cells were stained in 50 μl Perm/Wash buffer with anti-IL2-FITC (clone JES6-5H4, diluted 1/400), anti-IFNγ-APC (clone XMG1.2, diluted 1/200) and anti-TNFα-PE (clone MP6-XT22, diluted 1/700) antibodies, for 1 hour at 4° C. This panel of cytokines was selected based on the Th1 profile and pro-inflammatory cytokines that are known to be induced by the AS01 Adjuvant System. Cells were washed twice with the Perm/Wash buffer and resuspended in 220 μl PBS. Stained cells were analyzed by flow cytometry using a BD Fortessa Flow cytometer and the FlowJo software. All the antibodies and buffer used for the ICS are from BD Biosciences.

### Example 8—HA Mut 10 and HA Mut 23 Induce Functional HI Antibody Response Against Homologous and Post-Pandemic Heterologous H1N1 Strains at 14 Days Post Dose 2

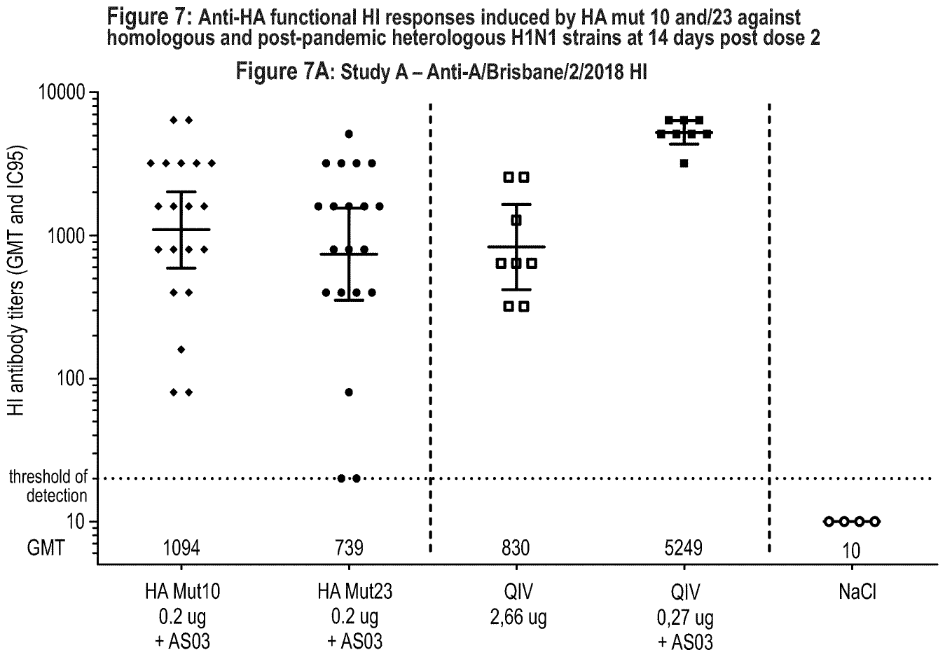

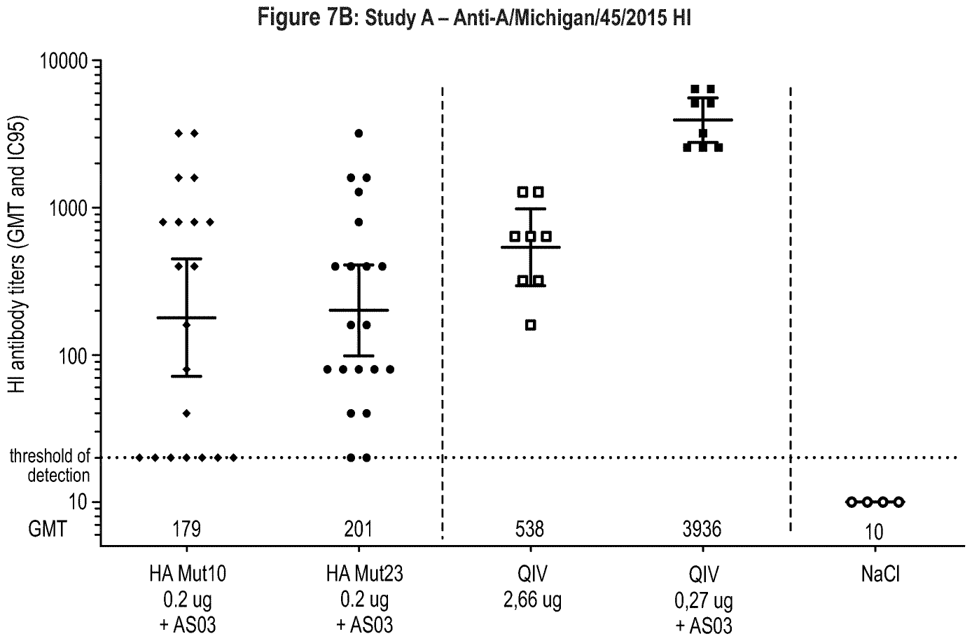

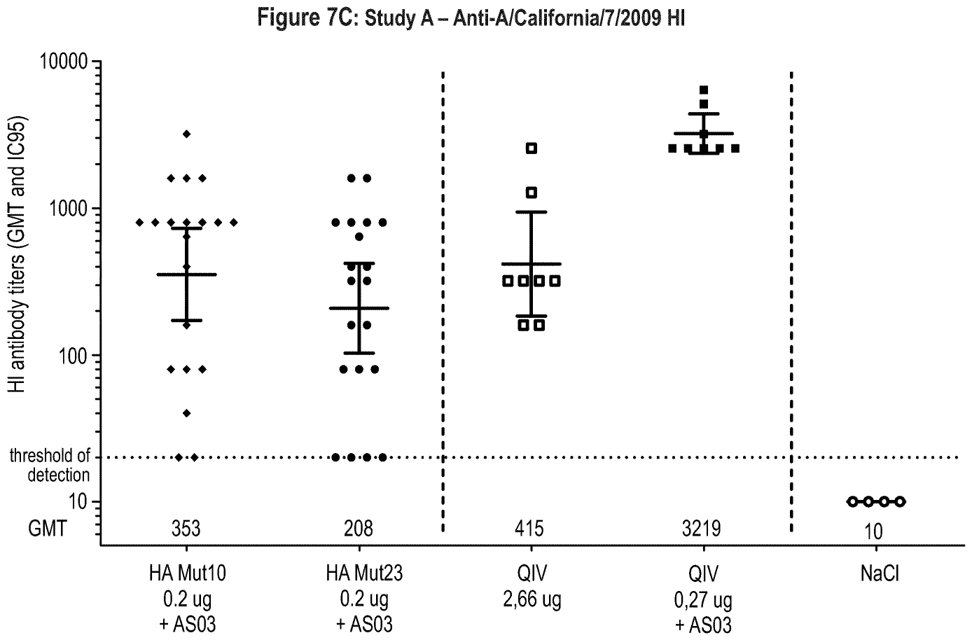

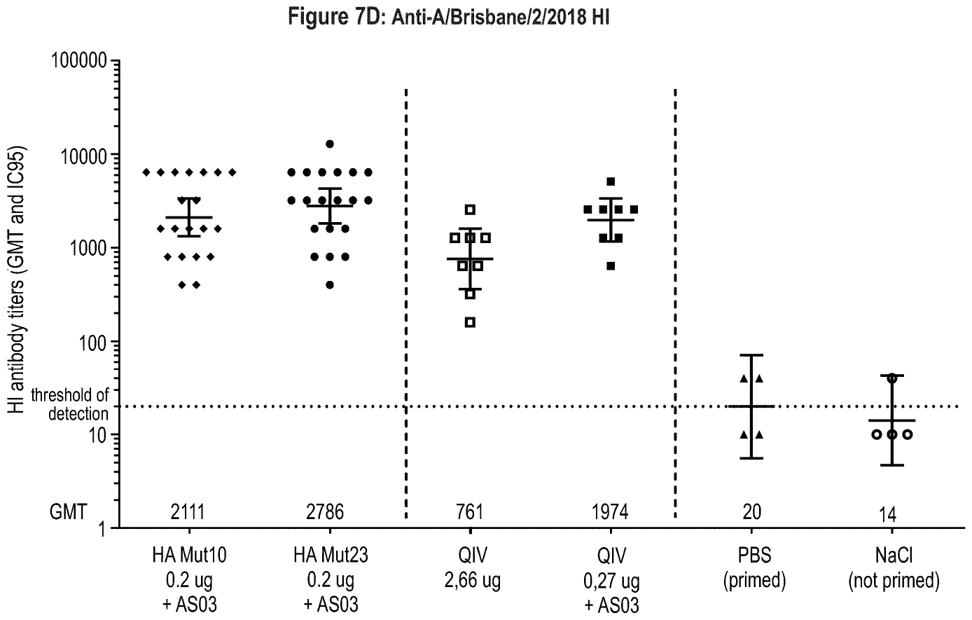

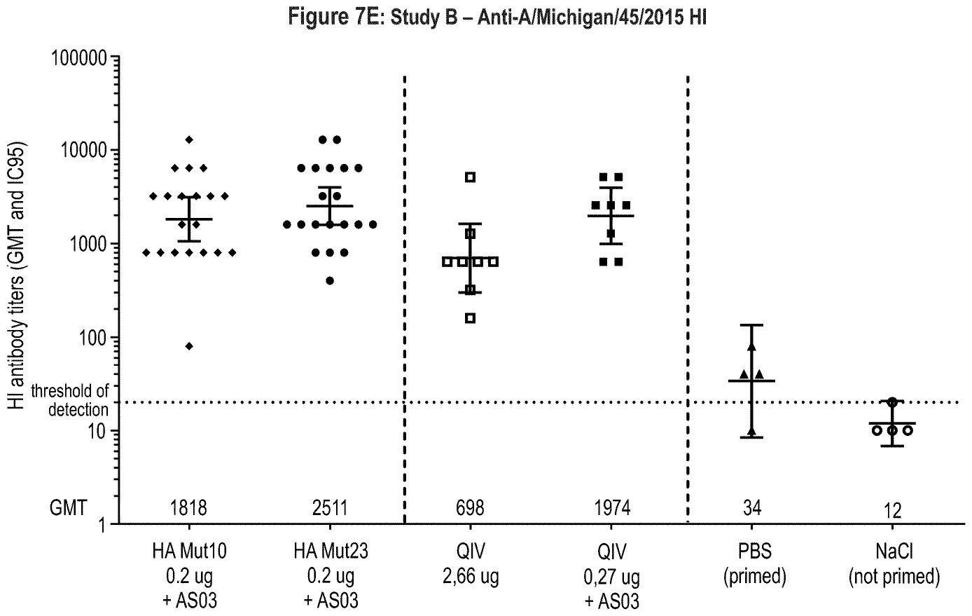

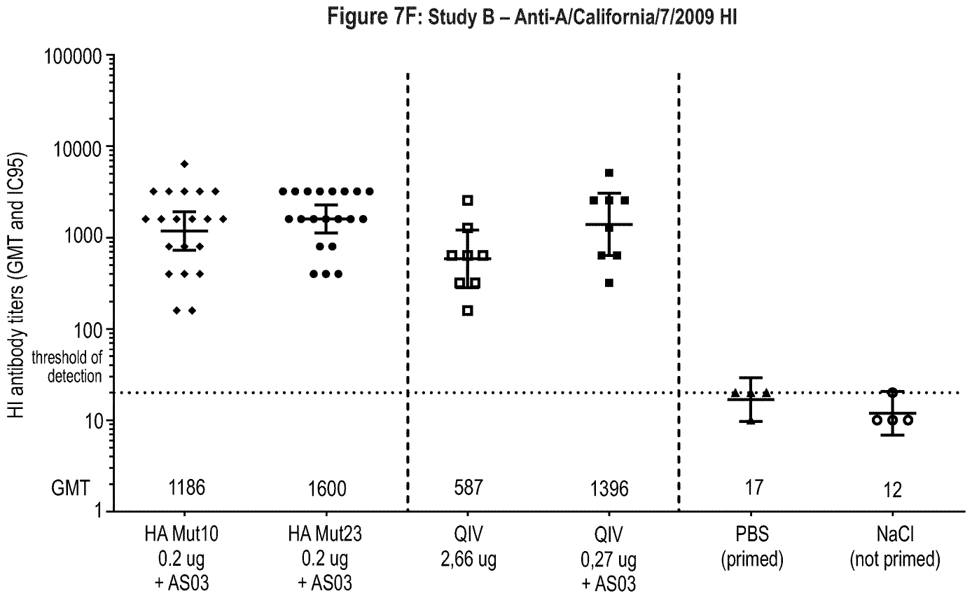

HI responses induced by HA mut 10 and HA mut 23 in a naïve (study A) and primed (study B) mouse model against homologous (A/Brisbane/2/2018) and post-pandemic heterologous (A/Michigan/45/2015 and Anti-A/California/7/2009) H1N1 strains measured at 14 days post second immunization are shown in FIG. 7. Individual titers values are shown together with the geometric mean titers (GMT) and 95% confidence intervals (95C1).

### Conclusions:

In addition to the induction of a homologous HI response (against A/Brisbane/2/2018), administration of HA mut 10 and HA mut 23 induced cross-reactive anti-HA response against post-pandemic heterologous (A/Michigan/45/2015 and Anti-A/California/7/2009) H1N1 strains in both naïve (study A) and primed (study B) animals. The variability of the HI responses induced by HA mut 10 and HA mut 23 was highly reduced when assessed in the primed model (study B). The measured HI titers were comparable (naïve and primed animals) or lower (naïve animals) with HA mut 10 and HA mut 23 compared to QIV (±AS03).

### Example 9—HA Mut 10 and HA Mut23 Induce Anti-HA Stem Binding and Functional Antibody Responses as Well as Neutralizing Antibody Responses Against Post-Pandemic Heterologous H1N1 Strains at 14 Days Post Dose 2

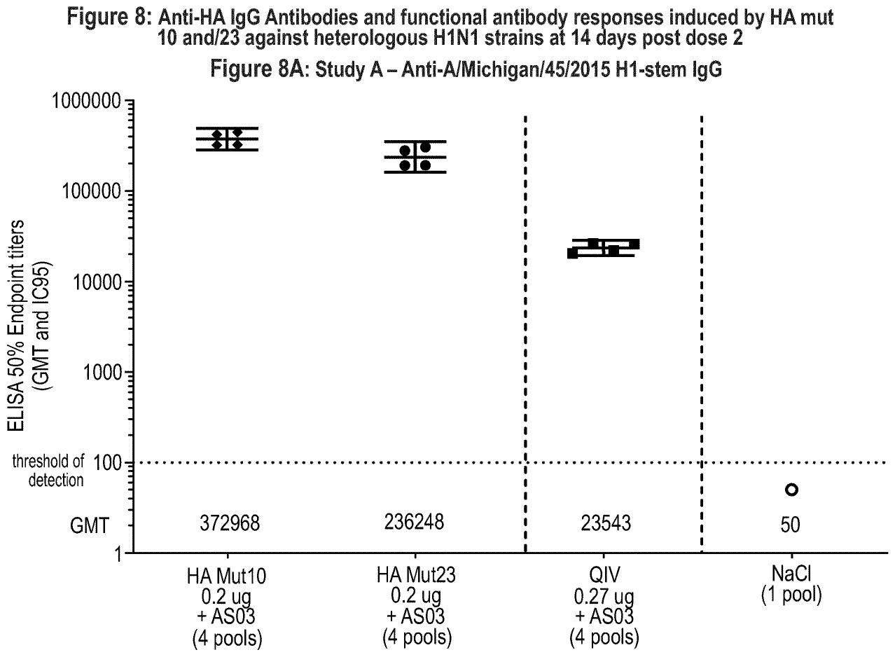

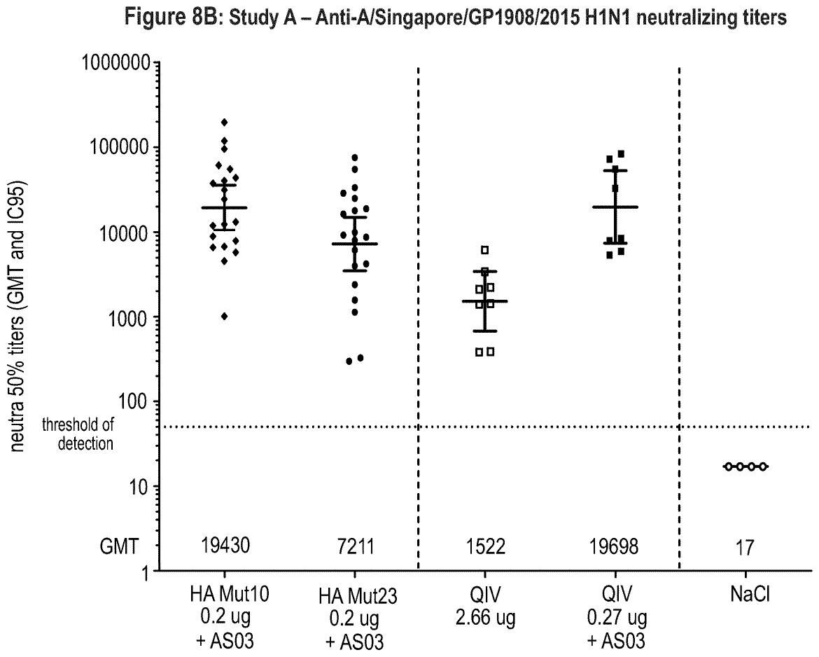

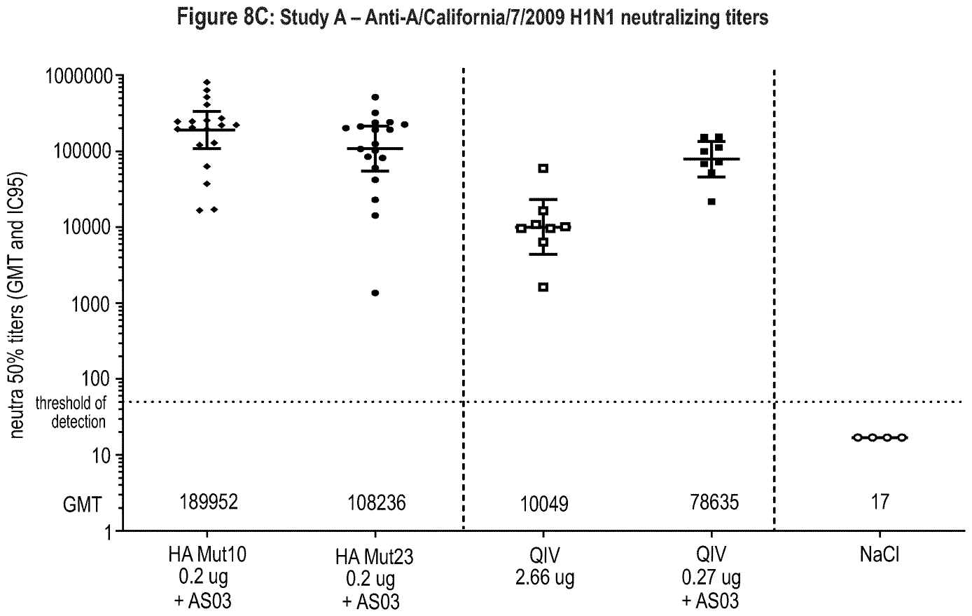

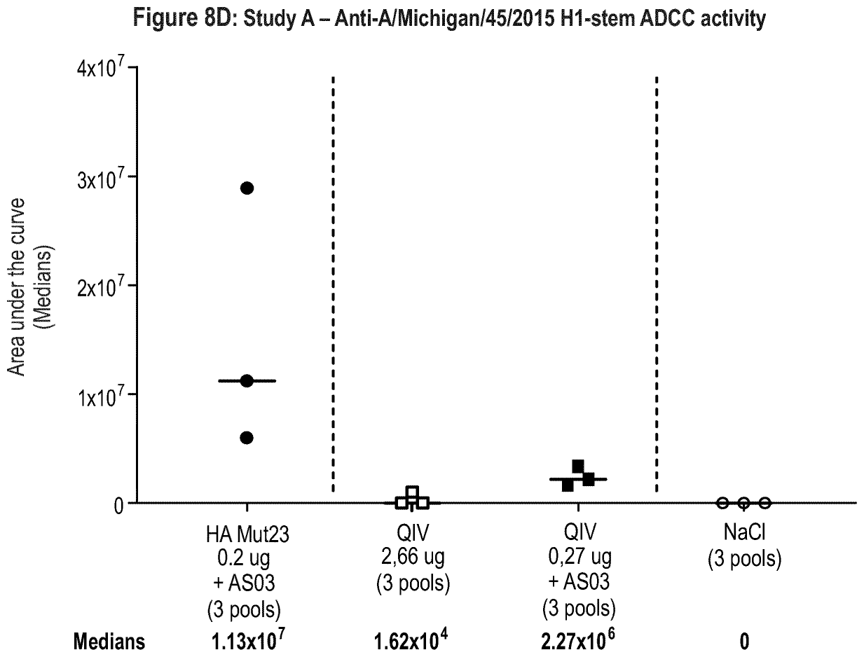

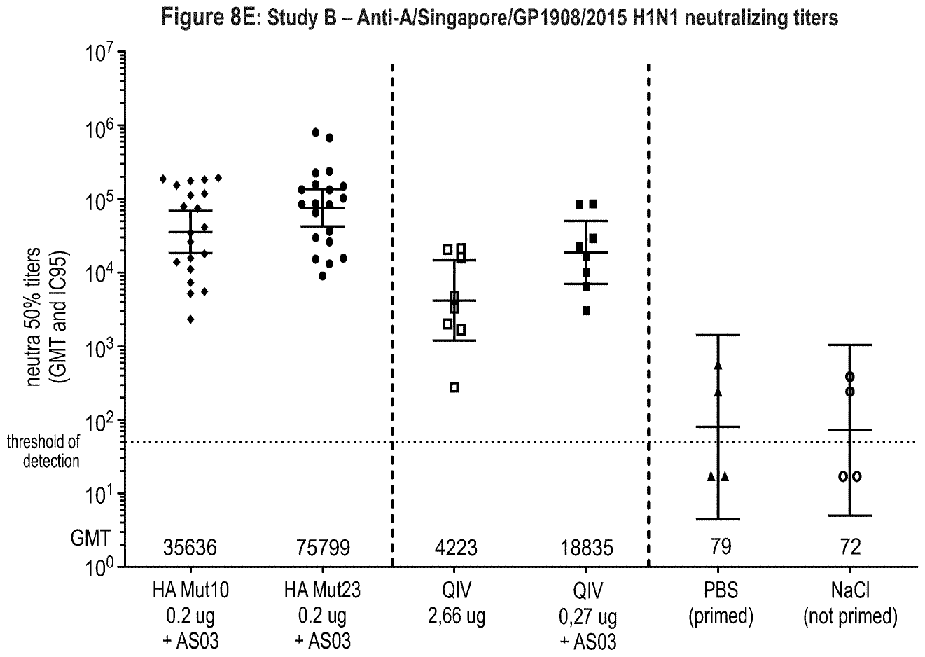

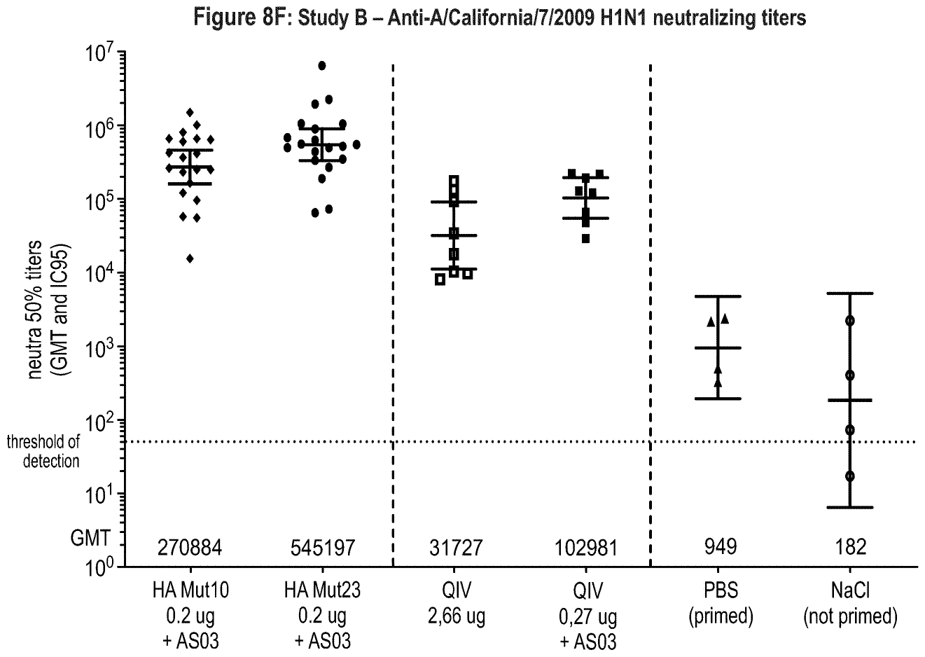

To characterize the induced antibody response, anti-H1 stem binding antibodies were measured by ELISA at 14 days post second immunization in naïve animals. The results from Study A are shown in FIG. 8. Pooled sera titers values are shown together with the geometric mean titers (GMT) and 95% confidence interval (95C1).

ADCC activity against A/Michigan/45/2015 stem was measured by ADCC Reporter Bioassay Promega at 14 days post second immunization. The results from Study A are shown in FIG. 8. Pooled sera AUC (Area under the curve) values are shown together with the medians.

Neutralizing antibodies against A/Singapore/GP1908/2015 H1N1 and A/California/7/2009 H1N1 strains were measured at 14 days post second immunization in Study A and B. The results from this assay are shown in FIG. 8. Individual titers values are shown together with geometric mean titers (GMT) and 95% confidence interval (95C1).

### Conclusions:

As the HI antibody response is solely directed against the head of the HA molecule, an anti-stem ELISA was performed to determine whether HA mut 10 and HA mut 23 can also induce stem specific antibodies. The stem only HA used were expressed on nanoparticles to ensure antigen stability while avoiding the use of a foldon, the presence of which could result in the detection of non-Flu but foldon-specific Ab responses.

The levels of stem-specific antibody titers detected in naïve mice immunized with HA mut 10 and HA mut 23 were substantially higher compared to the level induced by animals immunized with QIV. Furthermore, in contrast to the antibodies induced by QIV, anti-stem antibodies induced by HA mut 23 showed functional ADCC titers.

Neutralizing antibodies were also induced by HA mut 10 and 23 in both naïve and primed animals at comparable or slightly higher levels compared to animals immunized with adjuvanted QIV or unadjuvanted QIV respectively.

Altogether these data highlighted the capability of HA mut 10 and/or 23 to induce both stem- and head-specific antibody responses against a heterologous H1 strain with functional capacities comparable or greater than QIV.

### Example 10—HA Mut 10 and HA Mut 23 Induce Low H1-Stem Specific CD4 T Cell, but No CD8 T Cell Responses at 14 Days Post Dose 2

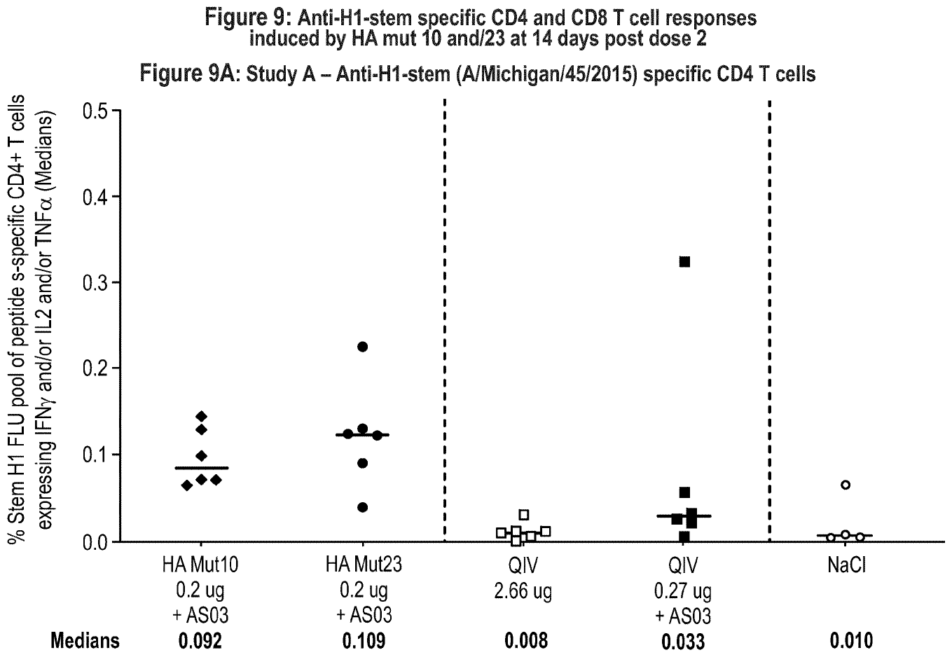

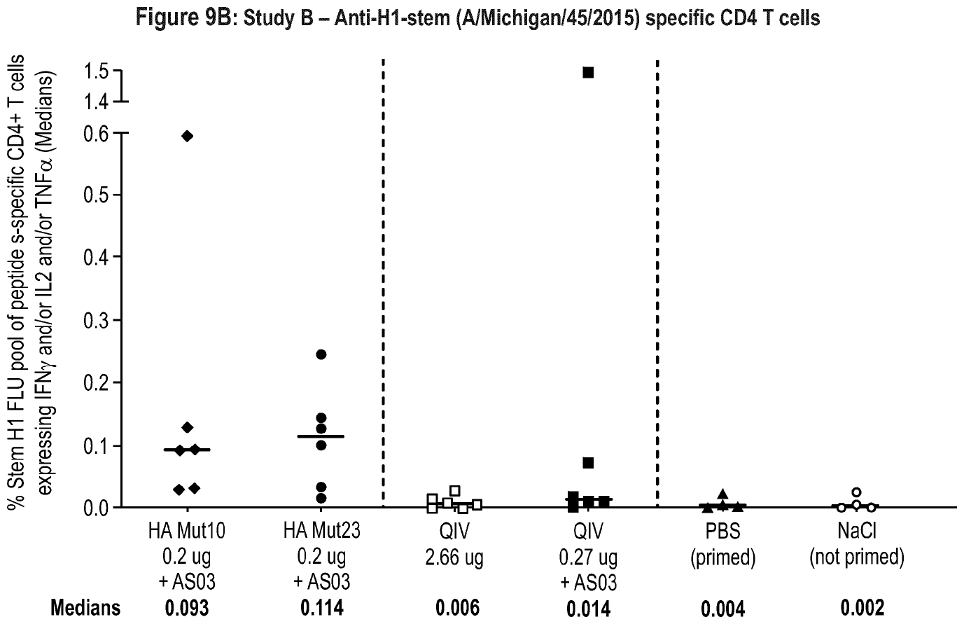

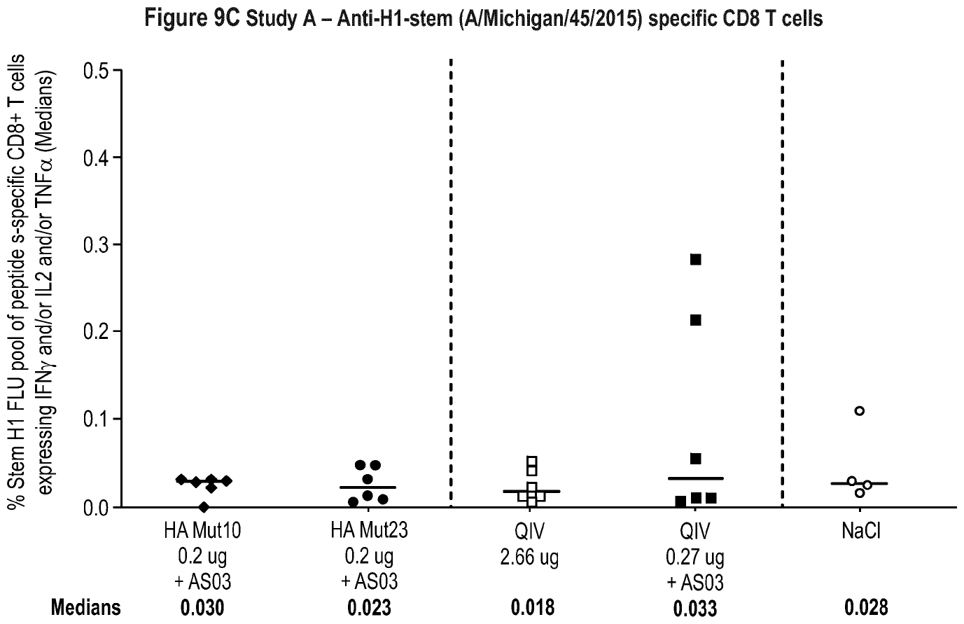

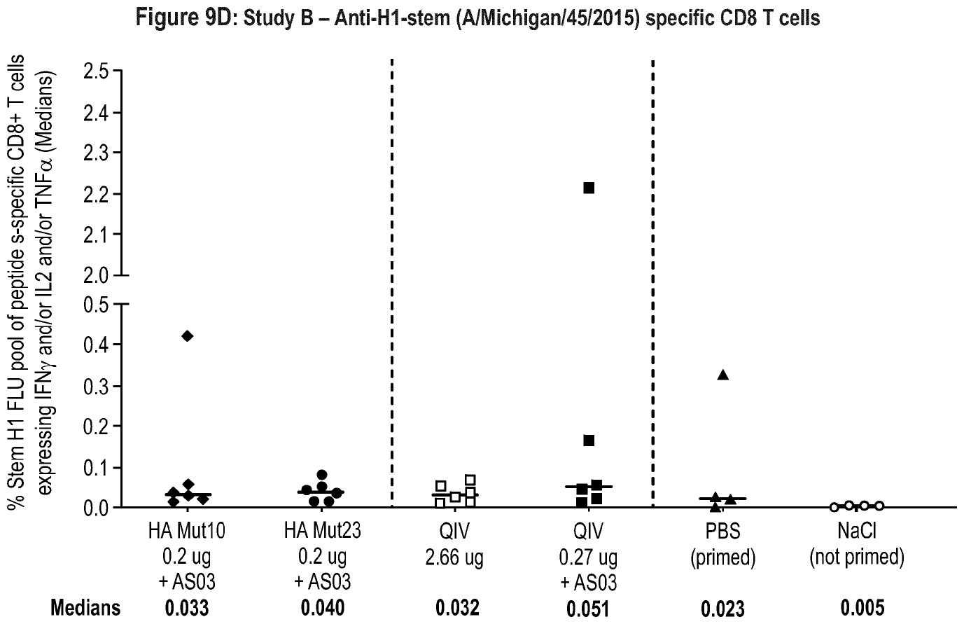

Anti-H stem specific CD4 and CD8 T cells were measured in a naïve (study A) and primed (study B) mouse model at 14 days post second immunization. The results from Study A and Study B are shown in FIG. 9. The frequencies of H1 Stem-specific CD4 or CD8 T cells expressing IFNγ and/or IL2 and/or TNFα are shown together with the medians.

### Conclusions:

Low % of H1 Stem (A/Michigan/45/2015) specific CD4 T cells expressing IFNγ and/or IL2 and/or TNFα were detected in both naïve (study A) and primed (study B) mouse model, but no induction of CD8 T cell could be detected in either model.

### Example 11—HA Mut 10 and HA Mut 23 Induce Broad Heterologous and Heterosubtypic HA-Binding Antibody Responses Against Pre-Pandemic Heterologous H1N1 and Heterosubtypic (H2N2, H5N1 and H9N2) Strains at 14 Days Post Dose 2

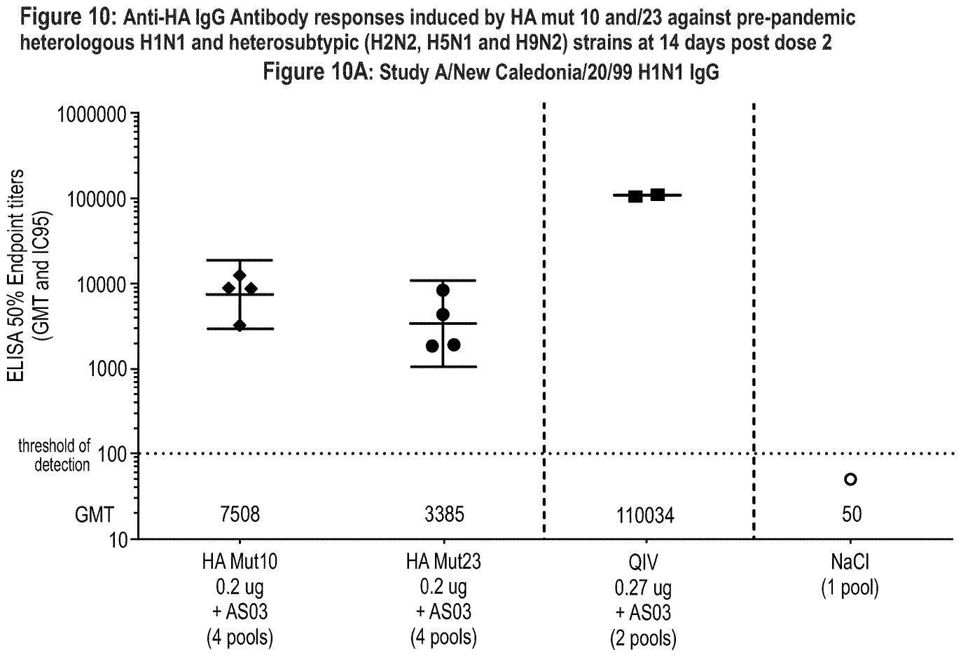

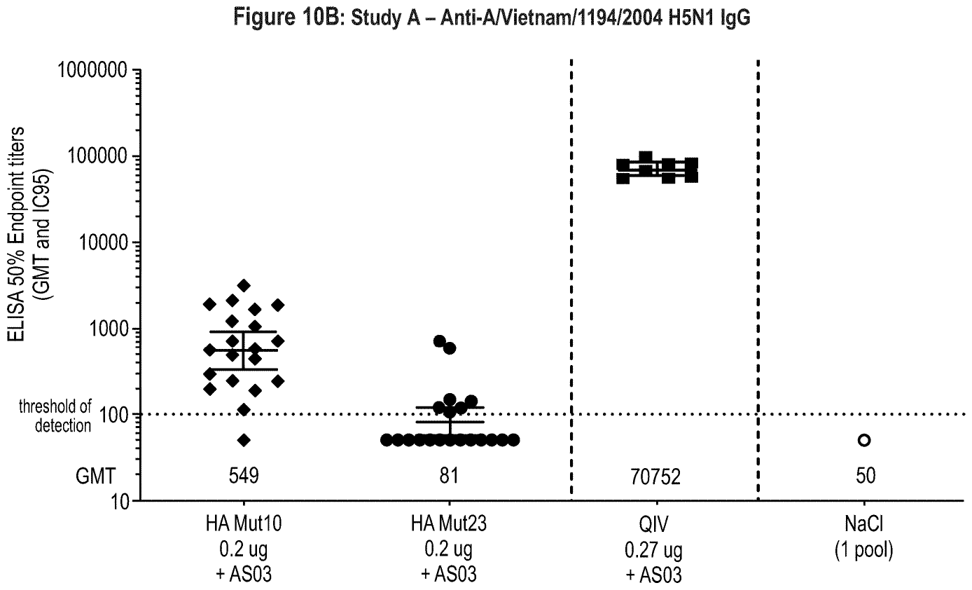

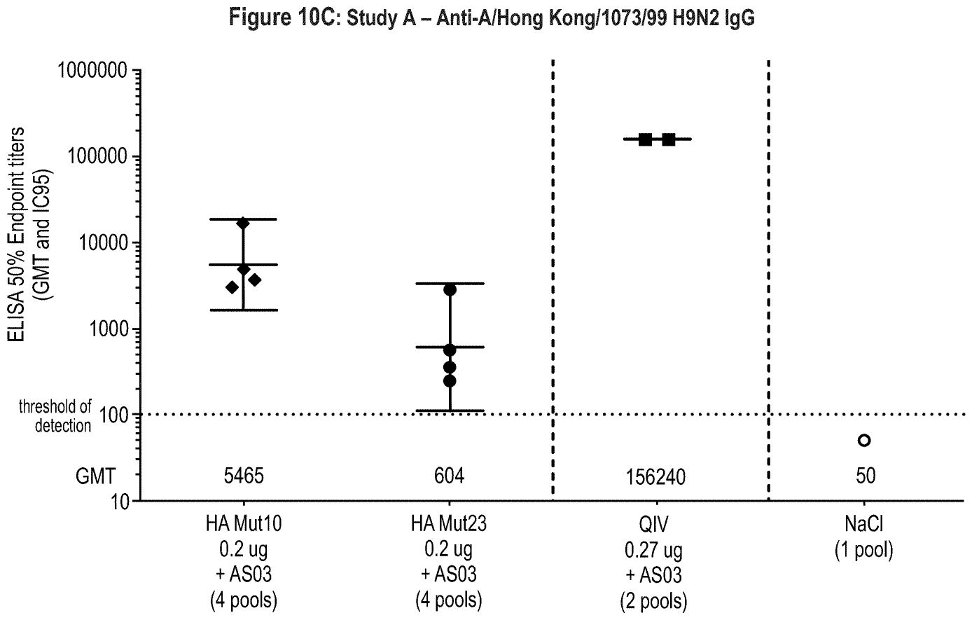

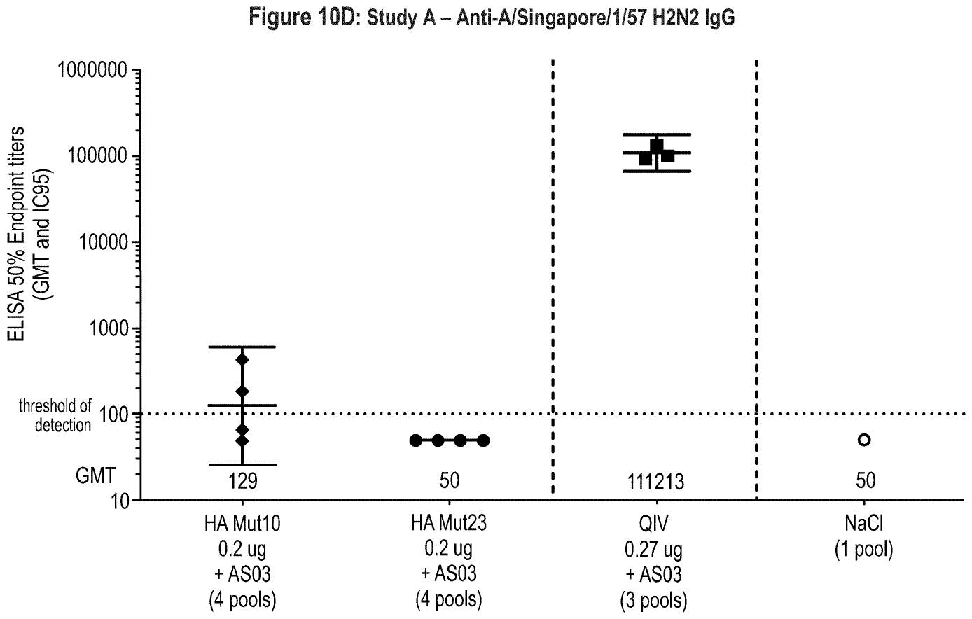

Anti-HA IgG binding antibodies induced by HA mut 10 and HA mut 23 in a naïve (study A) mouse model against pre-pandemic heterologous H1N1 and heterosubtypic (H2N2, H5N1 and H9N2) strains as measured by ELISA at 14 days post second immunization are shown in FIG. 10. Pooled sera (FIG. 10 A,C,D) and individual (FIG. 10 B) titers values are shown together with the geometric mean titers (GMT) and 95% confidence intervals (95C1).

### Conclusions:

High levels of cross-reactive IgG antibody titers directed against a pre-pandemic H1N1 strain (A/New Caledonia/20/99) and against a heterosubtypic H9N2 strain (A/Hong Kong/1073/99) were detected in naïve mice immunized with HA mut 10 and HA mut 23 (FIG. 10). Cross binding antibodies were also detected against a heterosubtypic H5N1 strain (A/Vietnam/1194/2004 H5N1) and a H2N2 strain (A/Singapore/1/57 H2N2), although at lower levels. As whole viruses were used as coating antigens, the antibody response measured in the QIV immunized animals is not specific to the HA antigens but also included responses against all other split flu components. Comparison with QIV-immunized mice is therefore not relevant in this assay.

### Example 12—HA Mut 10 and HA Mut 23 Cross Reactive Functional Antibody Responses Against an Heterosubtypic (H5N1) Strains at 14 Days Post Dose 2

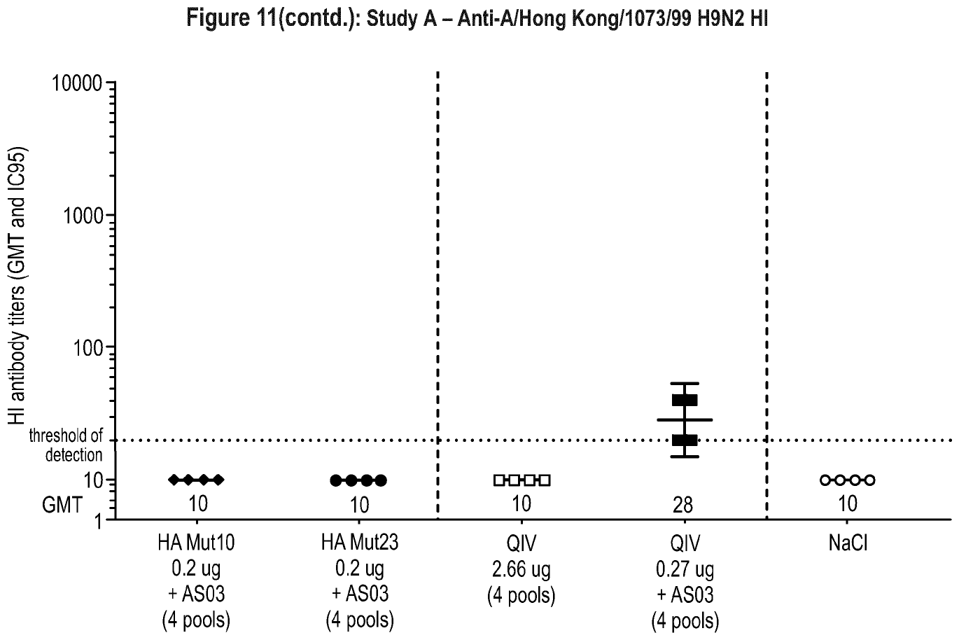

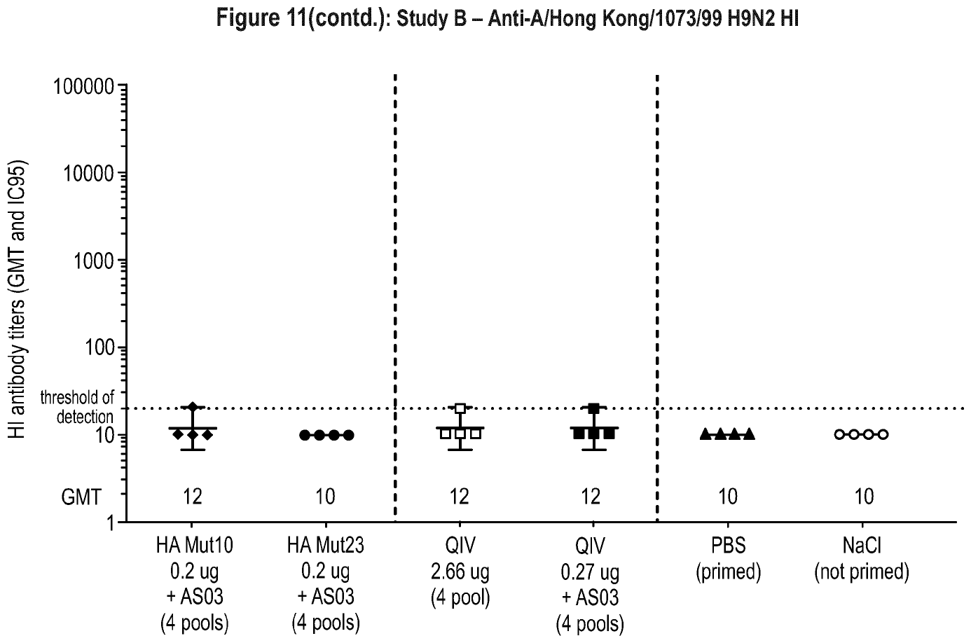

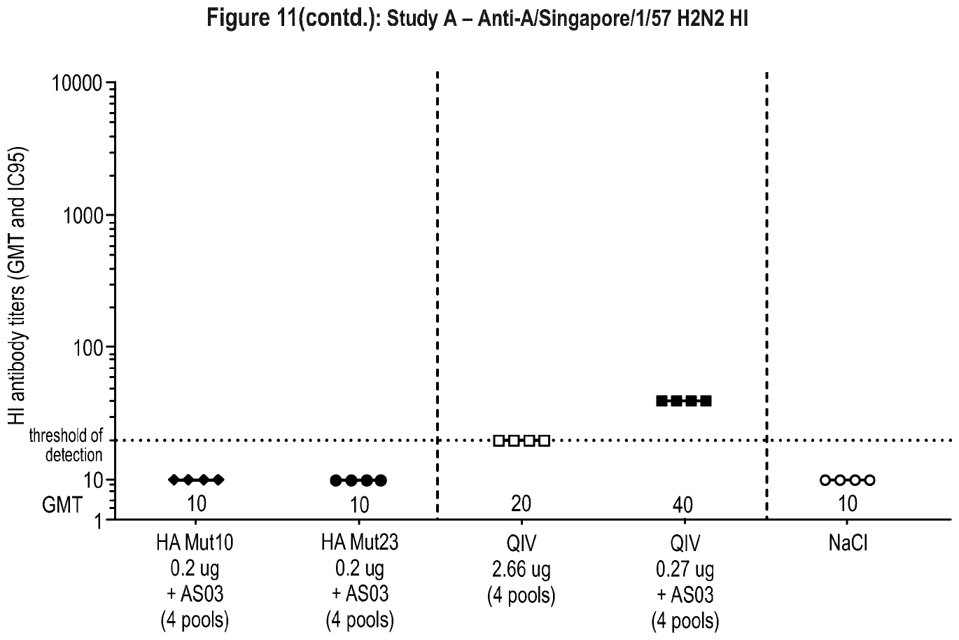

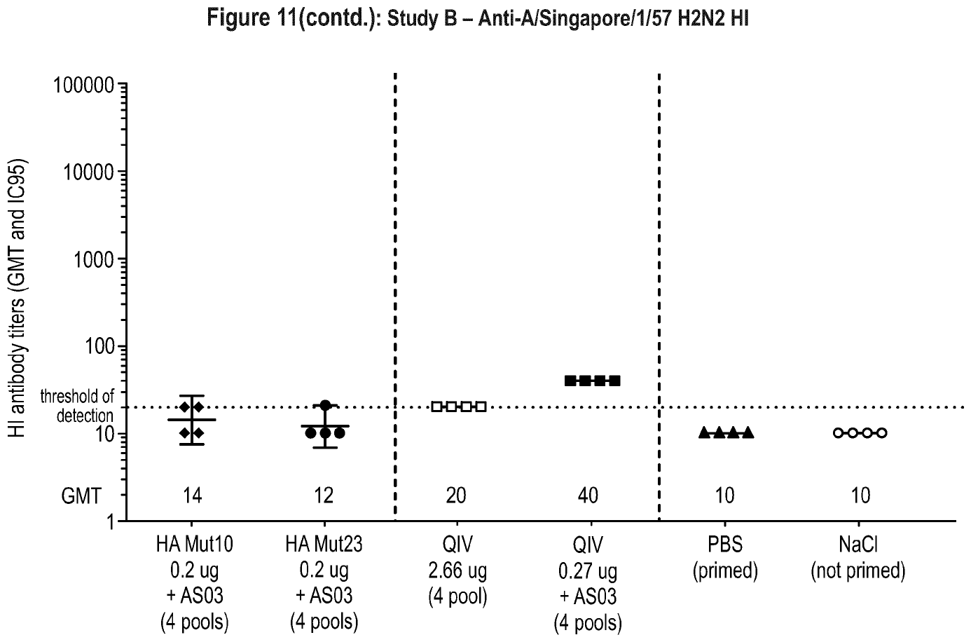

HI responses induced by HA mut 10 and HA mut 23 in a naïve (study A) and primed (study B) mouse model against pre-pandemic heterologous H1N1 and heterosubtypic (H2N2, H5N1 and H9N2) strains as measured at 14 days post second immunization are shown in FIG. 11. Pooled sera titers values are shown together with the geometric mean titers (GMT) and 95% confidence intervals (95C1).

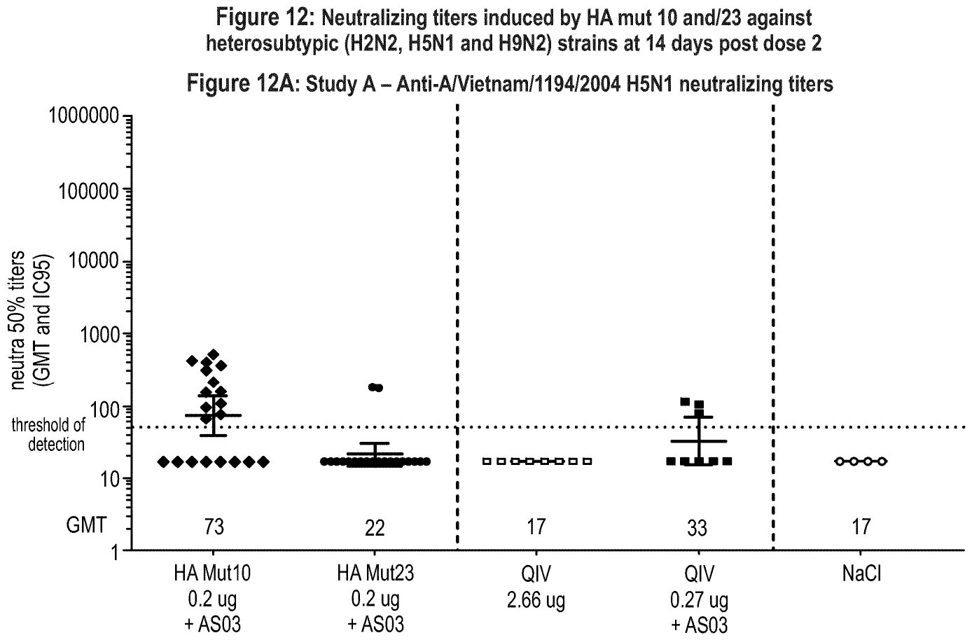

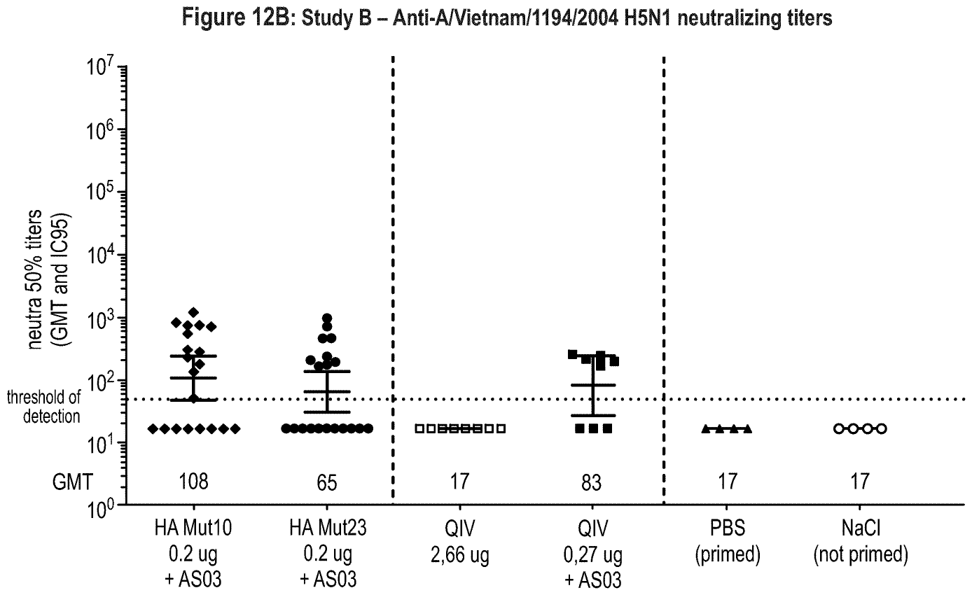

Neutralizing antibodies against Anti-A/Vietnam/1194/2004 H5N1 were measured at 14 days post second immunization in Study A and Study B and results from this assay are shown in FIG. 12. Values were geometric mean titers (GMT) and 95% confidence interval (95C1).

### Conclusions:

In contrast to the cross-reactive IgG responses that were observed, no HI responses were induced by HA mut 10 and/23 against a pre-pandemic heterologous H1N1 strain (A/New Caledonia/20/99) and against a heterosubtypic H9N2 strain (A/Hong Kong/1073/99), a heterosubtypic H5N1 strain (A/Vietnam/1194/2004 H5N1) and a H2N2 strain (A/Singapore/1/57 H2N2) at 14 days post dose 2.

However, even though higher in the primed model compared to the naïve model, neutralizing antibodies were also induced by HA mut 10 and 23 in both naïve and primed animals at comparable or slightly higher levels compared to animals immunized with adjuvanted QIV or unadjuvanted QIV respectively.

As the HI response is solely directed against the head of the HA molecule, these data suggest the induction of stem-based cross-functional antibody responses against heterosubtypic strains from the A1 group.

### Example 13—HA Mut 10 and HA Mut 23 Induce Limited Cross-Reactive Antibody Responses Against A2 Group (H3N2, H10-Stem) or B Lineages (B/Yam and B/Vic) Strains at 14 Days Post Dose 2

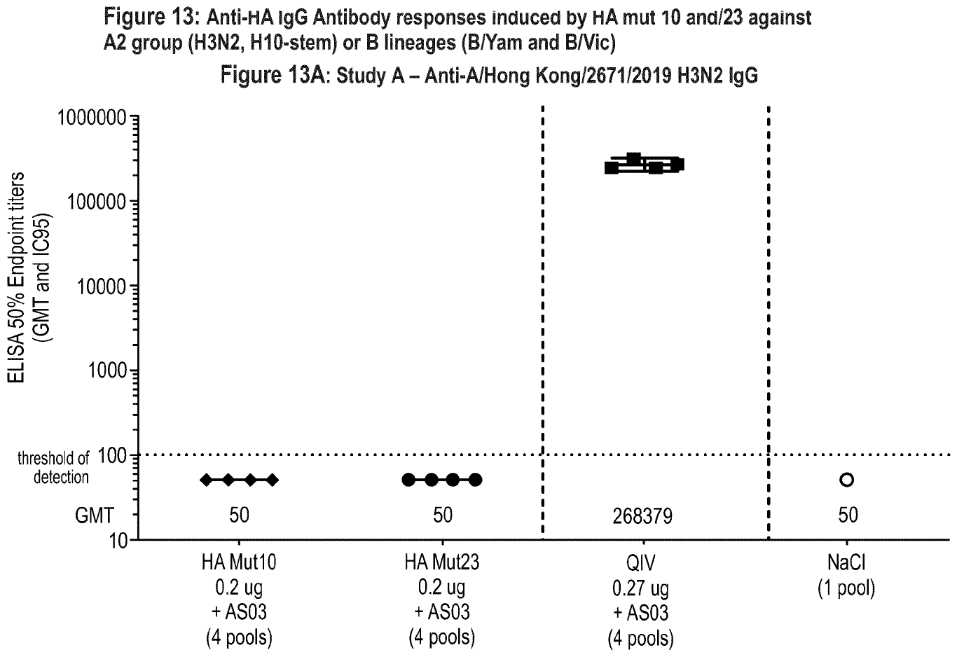

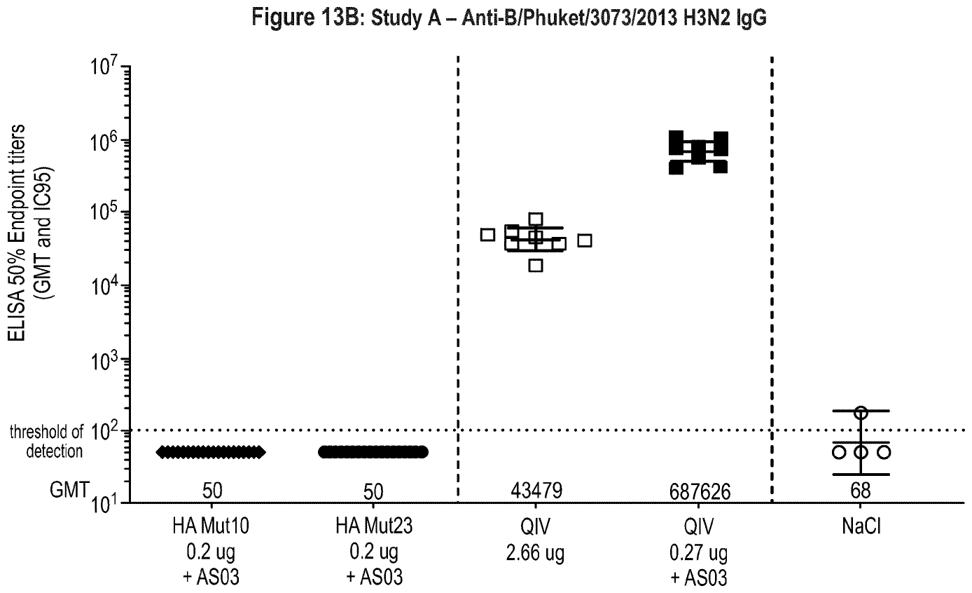

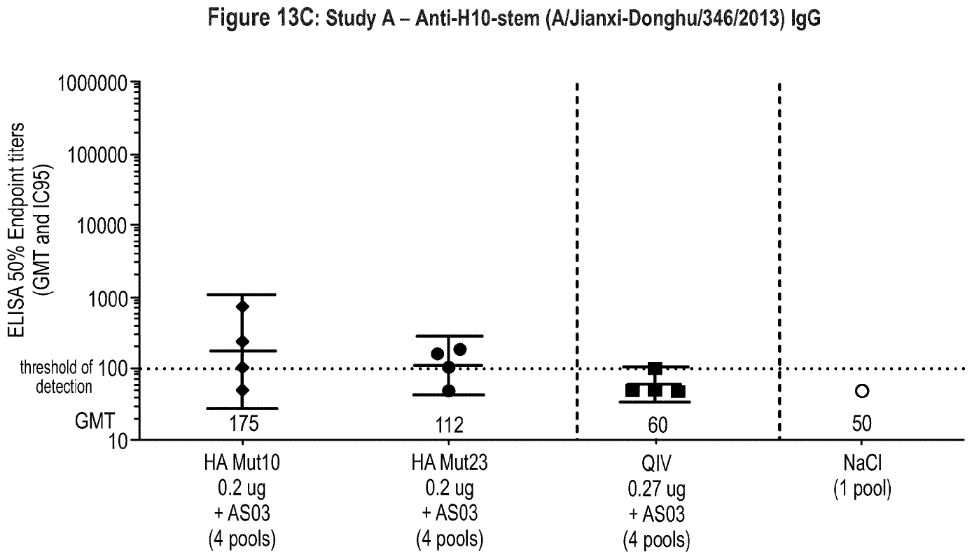

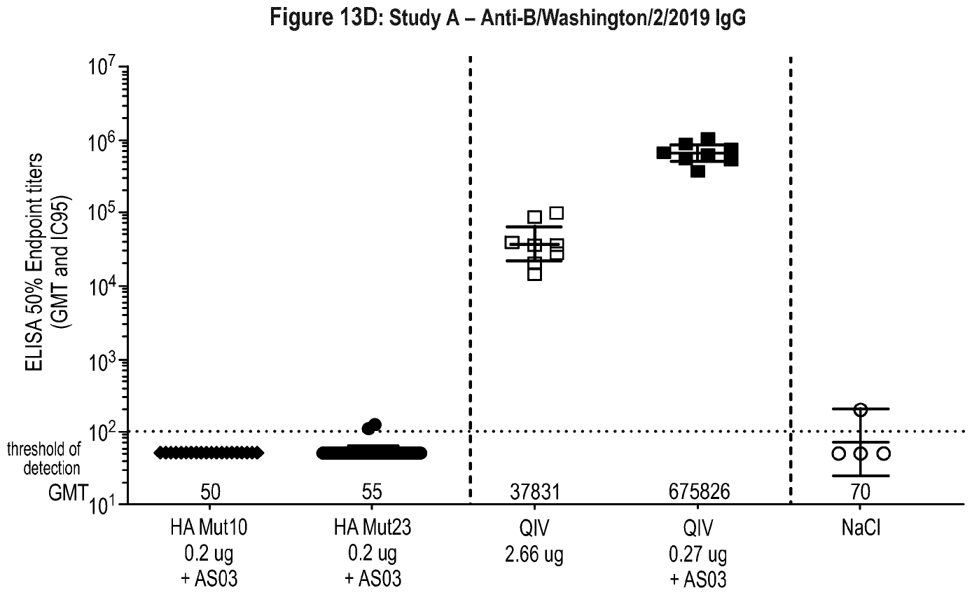

Anti-HA IgG binding antibodies induced by HA mut 10 and HA mut 23 in a naïve (study A) mouse model against A2 group (H3N2, H10-stem) or B lineages (B/Yam and B/Vic) strains measured by ELISA at 14 days post second immunization are shown in FIG. 13. Individual titers values are shown together with the geometric mean titers (GMT) and 95% confidence intervals (95C1).

### Conclusions:

While no cross-reactive IgG antibody titers directed against a H3N2 strain (A/Hong Kong/2671/2019 H3N2) were detected, low levels of cross-reactive anti-stem IgG were detected against H10 (A/Jianxi-Donghu/346/2013 stem). This highlighted the capability of HA mut 10 and/or 23 to induce A2 group cross-reactive antibodies against the stem portion of the HA to a comparable or higher level compared to the QIV group. No cross-reactive antibodies were detected against the tested B strains. As split flu viruses were used as coating antigens (except in the assessment of the H10 stem), the antibody response measured in the QIV immunized animals is not specific to the HA antigens but also included responses against all other split flu components. Comparison with QIV-immunized mice is therefore not relevant in this assay.

1. An immunogenic composition comprising a recombinant influenza A strain haemagglutinin (HA) antigen in trimeric form, the antigen comprising an ectodomain of HA, without a transmembrane or cytosolic domain, wherein the ectodomain comprises:
(i) a globular head domain; and
(ii) a stem domain having a coiled coil region, comprising one or more mutations in the coiled coil region that individually or together stabilise the HA ectodomain in trimeric prefusion form;
and wherein the recombinant HA optionally comprises a heterologous trimerization domain; together with a pharmaceutically acceptable carrier.

2. The immunogenic composition according to claim 1, wherein the recombinant HA has one or more amino acid substitutions in the coiled coil region compared to wild type.

3. The immunogenic composition according to claim 1, wherein the coiled coil region of the stem domain comprising one or more amino acid substitutions is from amino acid positions:
(a) 317 to 472, such as from 322 to 467 for H1; or (b) 342 to 473, such as from 347 to 468 for H2; or equivalent ranges for the coiled coil region in other HA A strains or subtypes.

4. The immunogenic composition according to claim 1, wherein the recombinant HA has one or more amino acid substitutions at one or more locations selected from:
(a) positions 322, 395, 431, 432, 436, 438, 439, 447, 449, 450, 453, 460, 464 and 467 of a HA group A1 subtype such as H1; or
(b) positions 347, 396, 399, 418, 428, 437, 440, 448, 451, 454, 465 and 468 of a group A2 subtype such as H3.

5. The immunogenic composition according to claim 1, wherein there are one or more amino acid substitutions at one or more locations selected from 395, 436 and 447 in H1 subtype or 396, 437 and 448 in H3 subtype or equivalent positions in other influenza A subtypes.

6. The immunogenic composition according to claim 1, wherein the recombinant HA contains R at position 422 in H1 or R at position 423 for H3.

7. The immunogenic composition according to claim 1, wherein the recombinant HA has at least one stabilising amino acid substitution selected from: K322R, K395M, G431C, F432C, W436D, Y438D, N439L, E447L, E449Q, R450W, D453L, K4601, E464F and R467M for H1 subtype, or at least one stabilising amino acid substitution selected from: 1347F, K396L/l/V/M, R399L/M/F/l/L, V418P, W437D, N4401, E448M/l/L/M/V, H451L, D454A, E465M and K468M for H3 subtype.

8. The immunogenic composition according to claim 1, wherein the one or more amino acid substitutions comprises or consists of an amino acid substitution at position 395/396 such as K395/396L/1/V/M.

9. The immunogenic composition according to claim 8, wherein the recombinant HA additionally comprises an amino acid substitution at position 436/437 such as W436/437D, and/or 447/448 such as E447/448M/l/L/M/V.

10. The immunogenic composition according to claim 1 wherein the recombinant HA has an amino acid sequence having at least 85% or at least 87% or at least 90% identity to an amino acid sequence selected from SEQ ID Nos: 3-8 and 17-42, with or without the signal sequence.

11. The immunogenic composition according to claim 1 wherein the HA comprises a foldon.

12. The immunogenic composition according to claim 1 wherein the HA ectodomain comprises all of the HA1 and all of the HA2 regions, without the transmembrane and cytosolic domains.

13. The immunogenic composition according to claim 1 wherein the stem domain is
(1) covalently linked to a heterologous trimerization domain; or
(2) covalently linked to a carrier protein or a nanoparticle; or
(3) not covalently linked to another amino acid molecule.

14. An immunogenic composition comprising an isolated polynucleotide such as DNA or mRNA, encoding the recombinant HA antigen of the immunogenic composition of claim 1, and a pharmaceutically acceptable carrier.

15. The immunogenic composition of claim 14 wherein the polynucleotide is in a nucleic acid delivery platform.

16. The immunogenic composition of claim 14 wherein the HA antigen comprises a transmembrane region such as an HA transmembrane region, with or without a HA cytosolic region.

17. A process for preparing an immunogenic composition according to claim 1, the process comprising:
(i) Expressing the recombinant HA antigen, from a polynucleotide sequence encoding the HA antigen fused to a heterologous trimerization domain e.g. foldon, in a eukaryotic cell;
(ii) Purifying recombinant HA trimers, from the cell supernatant;
(iii) Removing the trimerization domain;
(iv) Combining the recombinant HA timers with a pharmaceutically acceptable carrier.

18. A process for preparing an immunogenic composition comprising an HA ectodomain comprising one or more mutations in the coiled coil region that individually or together stabilise the HA ectodomain in trimeric prefusion form, the method comprising:
(i) expressing recombinant HA antigen, from a polynucleotide sequence encoding it with or without a trimerization domain;
(ii) purifying trimeric recombinant HA, from the cell supernatant;
(iii) optionally removing the trimerization domain if present;
(iv) combining the recombinant HA trimers with a pharmaceutically acceptable carrier.

19. A method for prevention of and/or vaccination against influenza A strain infection or disease, comprising the administration of the immunogenic composition of claim 14 to a person in need thereof, such as a person identified as being at risk of influenza virus infection or disease.

20. A method of generating an immune response against influenza A strain, comprising administering a recombinant influenza A strain HA antigen of claim 14, to a human subject.


SEQUENCE LISTING (73 sequences)

SEQ ID NO: 1 - protein, 566 aa, Influenza A
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 2 - protein, 566 aa, Influenza A
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 3 - protein, 554 aa, synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNMVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVGYIPEAPRDGQAYVRKDGEWVLLSTFLGHHHHHH

SEQ ID NO: 4 - protein, 566 aa, synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNMVNSVIEKMNTQFTAVGKEFNHLEKRIENLNKKVDDGFLDIWTYNAELLVLLLNQWTLLYHDSNVKNLYEKVRNQLKNNAKEIGNGCFEFYHKCDNTCMESVKNGTYDYPKYSEEAKLNREKIDGVGSENLYFQGGSKGYIPEAPRDGQAYVRKDGEWVLLSTFLGHHHHHH

SEQ ID NO: 5 - protein, 566 aa, synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVINLYEKVRNQLKNNAKEIGNGCFEFYHKCDNTCMESVKNGTYDYPKYSEEAKLNREKIDGVGSENLYFQGGSKGYIPEAPRDGQAYVRKDGEWVLLSTFLGHHHHHH

SEQ ID NO: 6 - protein, 566 aa, synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPRYVKSTKLRLATGLRNVPSIQSRGLFGAIAGFIEGGWTGMVDGWYGYHHQNEQGSGYAADLKSTQNAIDKITNMVNSVIEKMNTQFTAVGKEFNHLEKRIENLNKKVDDGFLDIDTYNAELLVLLLNERTLDYHDSNVKNLYEKVRNQLKNNAKEIGNGCFEFYHKCDNTCMESVKNGTYDYPKYSEEAKLNREKIDGVGSENLYFQGGSKGYIPEAPRDGQAYVRKDGEWVLLSTFLGHHHHHH

SEQ ID NO: 7 - protein, 566 aa, synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPRYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIDTYLAELLVLLENQWTLLYHDSNVKNLYEKVRNQLKNNAKEIGNGCFEFYHKCDNTCMESVKNGTYDYPKYSEEAKLNREKIDGVGSENLYFQGGSKGYIPEAPRDGQAYVRKDGEWVLLSTFLGHHHHHH

SEQ ID NO: 8 - protein, 566 aa, synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPRYVKSTKLRLATGLRNVPSIQSRGLFGAIAGFIEGGWTGMVDGWYGYHHQNEQGSGYAADLKSTQNAIDKITNMVNSVIEKMNTQFTAVGKEFNHLEKRIENLNKKVDDGFLDIDTYLAELLVLLLNQWTLLYHDSNVKNLYEKVRNQLKNNAKEIGNGCFEFYHKCDNTCMESVKNGTYDYPKYSEEAKLNREKIDGVGSENLYFQGGSKGYIPEAPRDGQAYVRKDGEWVLLSTFLGHHHHHH

SEQ ID NO: 9 - protein, 27 aa, Bacteriophage T4
GYIPEAPRDGQAYVRKDGEWVLLSTFL

SEQ ID NO: 10 - protein, 17 aa, Influenza A
MKAILVVLLYTFTTANA

SEQ ID NO: 11 - DNA, 1668 bp, synthetic construct
  source 1..1668: mol_type = other DNA
atgaaagctatcctcgtggtgctgctgtacaccttcaccaccgccaacgctgacacactgtgcatcggataccatgccaacaattccaccgacacagtggataccgtgctggagaaaaacgtgaccgtcacccactccgtcaatctgctcgaggacaagcacaacggaaagctctgcaagctgggcggagtggctcctctccatctgggaaagtgcaacattgccggctggattctgggaaaccccgagtgcgagtctctgtccaccgctaggagctggagctatatcgtcgaaacctccaactccgacaacggcacatgctaccccggcgacttcattaactacgaggaactgagagagcagctcagctccgtgtccagctttgagaggtttgagatcttccccaagaccagcagctggcccaaccacgacagcaacaaaggcgtcaccgccgcttgccctcacgctggcgccaaatccttctacaaaaatctgatctggctcgtcaagaagggcaatagctaccccaagctcaaccagacctacatcaacgacaaaggcaaagaggtgctcgtcctctggggaatccaccaccctcctaccacagccgaccagcagtctctgtaccagaacgccgacgcctatgtctttgtgggaacctctaggtactccaagaagttcaagcccgagatcgccacaaggcctaaagtgagagaccaagagggaaggatgaactactactggacactggtggaacccggcgataagatcacctttgaggccaccggcaacctcgtggtccccagatacgctttcaccatggagaggaacgccggcagcggaatcatcatcagcgatacacccgtgcacgactgtaacaccacatgccagaccgccgagggcgccattaacaccagcctccccttccagaacgtgcaccccgtcacaatcggcaagtgccccaagtacgtgaagagcacaaagctgagactcgctaccggactgaggaacgtgccttccatccaaagcagaggactgtttggagccatcgccggcttcatcgaaggaggctggaccggcatggtggatggatggtacggctaccaccaccaaaacgaacaaggctccggatacgccgccgacctcaagtccacccagaatgccatcgacaaaatcaccaacatggtgaactccgtgatcgagaagatgaacacccagttcaccgctgtgggcaaggagttcaaccacctcgagaagaggatcgaaaacctcaacaagaaagtggacgacggatttctggacatctggacctataacgccgaactgctcgtgctgctggaaaacgagagaacactggactaccacgactccaatgtgaagaatctgtacgagaaggtgaggaaccagctgaagaacaacgccaaggagatcggcaacggctgcttcgagttctaccacaaatgcgacaacacatgcatggagtccgtcaagaacggaacctacgactatcccaagtacagcgaggaggccaaactgaacagagagaagattgacggcgtgggctacatccccgaggctcctagagacggccaagcctacgtgaggaaggacggagagtgggtgctgctgtccacctttctgggacaccaccatcatcaccactgatga

SEQ ID NO: 12 - DNA, 1704 bp, synthetic construct
  source 1..1704: mol_type = other DNA
atgaaggctattctggtggtgctgctgtacaccttcaccaccgccaatgccgacacactgtgcattggctaccacgccaacaacagcaccgatacagtggacacagtcctcgagaagaacgtgaccgtcacacacagcgtgaatctgctcgaggacaagcacaacggaaagctctgtaagctgggaggagtcgcccctctgcatctgggcaagtgtaacatcgccggctggattctgggcaaccccgagtgtgaatctctgtccaccgccagaagctggagctacatcgtggaaacaagcaacagcgacaacggcacatgctaccccggcgacttcatcaactacgaagagctgagagagcagctgtccagcgtcagcagcttcgagagattcgagatctttcccaagaccagcagctggcccaatcacgattccaacaagggagtcaccgctgcttgcccccacgctggcgctaagtccttttacaagaacctcatttggctcgtgaagaagggcaactcctatcccaagctcaatcagacatatattaacgataaaggaaaggaagtgctggtgctctggggcatccaccaccctcccaccaccgctgaccaacagagcctctaccagaatgctgacgcctatgtctttgtgggaacctctaggtactccaagaagttcaagcccgagattgctacaagacccaaggtgagggaccaagagggaagaatgaactactactggacactggtggagcccggcgataagattaccttcgaggccaccggcaatctggtcgtccccagatatgccttcaccatggagagaaacgccggcagcggaatcattatcagcgacacccccgtgcatgattgcaacacaacatgccaaaccgccgagggcgccattaacacctccctcccctttcaaaacgtccaccccgtgacaatcggcaagtgccccaagtacgtgaaatccaccaagctgaggctggccaccggactgagaaacgtgccctccattcagagcagaggactgtttggagccattgccggctttattgagggcggctggaccggaatggtcgatggctggtacggctatcatcaccaaaacgagcaaggctccggatatgctgctgatctgaagtccacacaaaacgccatcgataagatcaccaacatggtgaacagcgtgatcgagaagatgaacacccagttcacagccgtgggcaaggaatttaaccacctcgagaaaaggatcgagaacctcaacaaaaaggtcgacgacggcttcctcgacatttggacctacaacgctgagctgctcgtgctgctgctgaaccagtggacactgctctatcacgactccaacgtgaagaatctgtatgagaaggtgagaaaccaactgaagaacaacgctaaggagatcggaaacggctgcttcgagttttaccacaagtgcgacaacacatgcatggagtccgtgaaaaacggcacatacgactaccccaagtattccgaggaagccaagctcaatagagaaaagatcgatggcgtcggcagcgagaatctgtattttcaaggcggctccaagggctatattcccgaggctcctagagacggacaagcctatgtgaggaaggatggcgaatgggtgctcctctccacatttctgggccaccaccaccatcatcactgatga

SEQ ID NO: 13 - DNA, 1704 bp, synthetic construct
  source 1..1704: mol_type = other DNA
atgaaggctattctggtcgtgctgctgtacaccttcaccacagccaacgccgatacactgtgtatcggctaccacgccaacaacagcaccgatacagtcgataccgtgctggagaagaacgtgaccgtcacccattccgtgaatctgctggaggacaagcacaatggcaagctgtgcaagctgggcggagtggcccctctgcatctgggcaagtgtaatatcgccggatggattctgggaaaccccgagtgtgaatctctgtccaccgctagaagctggtcctacattgtggagacctccaattccgacaatggcacatgctaccccggcgattttatcaactacgaggagctgagagaacaactgagcagcgtctcctcctttgagagattcgagatctttcccaagaccagcagctggcccaatcacgattccaacaagggagtcaccgctgcttgcccccacgctggcgctaagtccttttacaagaacctcatttggctcgtgaagaagggcaactcctatcccaagctgaatcaaacatatatcaacgacaagggcaaggaagtcctcgtgctgtggggaatccaccaccctcctacaacagccgaccaacagtctctgtatcagaacgccgacgcctatgtgttcgtgggaacctctaggtactccaagaagttcaagcccgagattgctacaaggcccaaggtgagagaccaagagggaagaatgaattactactggacactggtggagcccggcgataagatcaccttcgaagccaccggcaacctcgtggtgcctaggtacgccttcaccatggagagaaacgctggcagcggcatcatcatctccgacacacccgtgcatgactgcaacacaacatgccaaacagccgagggcgccattaacacctccctcccctttcagaacgtgcaccccgtgacaatcggcaagtgccccaagtacgtgaaatccacaaagctgaggctggccaccggactgagaaatgtgcccagcatccagtctaggggactgtttggagctatcgccggcttcattgaaggcggctggaccggaatggtggacggatggtacggctaccaccaccagaatgaacaaggctccggatacgctgctgatctgaagagcacccaaaacgccattgacaagatcaccaacaaggtcaactccgtcatcgagaagatgaacacccagtttaccgccgtgggcaaggaatttaaccacctcgagaaaaggatcgagaacctcaacaaaaaggtcgacgacggctttctcgacatttggacctacaacgccgagctgctggtgctgctcgagaatgagagaacactggactaccacgactccaatgtcatcaatctgtatgagaaggtgaggaaccagctcaagaacaacgctaaggagatcggaaacggctgcttcgagttttatcacaagtgcgataacacatgcatggagtccgtgaaaaacggcacatacgactatcccaagtactccgaagaggctaagctcaatagagagaagatcgatggcgtcggcagcgagaatctctatttccaaggcggcagcaagggctatatccccgaggcccctagagatggccaagcctatgtgaggaaggacggcgaatgggtgctgctgagcaccttcctcggccatcaccatcaccaccactgatga

SEQ ID NO: 14 - DNA, 1704 bp, synthetic construct
  source 1..1704: mol_type = other DNA
atgaaagccattctggtggtgctgctgtacacctttaccaccgccaatgccgatacactgtgcatcggctaccacgccaataactccaccgacaccgtggacacagtgctggagaagaacgtgaccgtgacacacagcgtgaacctcctcgaggacaagcacaacggaaagctgtgcaagctcggcggagtggcccctctgcatctgggaaagtgtaacattgccggctggattctgggaaaccccgagtgtgaatctctgagcaccgctagaagctggagctacattgtggagaccagcaacagcgacaacggaacatgctaccccggcgactttatcaattacgaggaactgagggagcagctgtcctccgtgagctcctttgagaggttcgagatctttcccaagaccagcagctggcccaatcacgactccaacaaaggagtgaccgctgcttgccctcacgccggcgctaagagcttctacaaaaacctcatctggctggtgaagaagggcaactcctaccccaagctgaaccaaacctacatcaatgacaaaggcaaggaggtcctcgtgctctggggaatccaccaccctcccacaaccgctgaccaacagagcctctaccagaacgctgacgcctatgtctttgtgggaacctctaggtactccaagaagttcaagcccgagattgctacaaggcccaaggtgagagaccaagagggaagaatgaattactactggacactcgtggagcccggcgacaaaatcaccttcgaagccaccggcaacctcgtggtccccagatacgccttcaccatggagaggaacgctggctccggcatcatcattagcgacacacccgtgcacgactgcaatacaacatgccaaacagctgagggcgccatcaatacctctctgccctttcagaacgtgcaccccgtgacaatcggcaaatgccctaggtacgtgaagagcaccaagctcagactcgccaccggactgaggaacgtgccctccatccagtctaggggactgtttggagccattgccggctttattgagggcggctggaccggaatggtggacggatggtacggctaccaccaccagaatgaacaaggctccggatacgccgctgatctgaagagcacacagaatgccattgataagattaccaacatggtgaacagcgtgatcgagaagatgaacacccagttcaccgccgtcggcaaggaatttaaccacctcgagaaaaggatcgagaacctcaacaaaaaggtcgacgacggcttcctcgacattgacacctacaacgctgagctgctggtgctgctgctgaatgagagaacactggactaccacgatagcaatgtcaagaatctgtatgaaaaggtgagaaaccaactgaagaacaacgccaaggagatcggaaacggctgcttcgagttttatcacaagtgcgataacacatgcatggagtccgtgaaaaacggcacctacgactatcccaagtactccgaggaggctaagctcaatagagaaaagatcgacggcgtcggaagcgaaaacctctactttcaaggcggcagcaagggctatatccccgaggctcctagagatggacaagcctatgtgaggaaggacggcgaatgggtcctcctcagcacctttctgggacaccaccaccatcaccactgatga

SEQ ID NO: 15 - DNA, 1704 bp, synthetic construct
  source 1..1704: mol_type = other DNA
atgaaggctattctggtcgtgctgctctacacctttaccaccgccaatgccgatacactgtgcatcggctatcacgccaacaacagcaccgataccgtcgacaccgtgctggagaagaacgtgaccgtcacacacagcgtgaatctgctggaggacaagcacaatggcaagctgtgcaagctgggcggagtggcccctctgcatctgggcaagtgtaatattgccggatggattctgggcaaccccgagtgcgagtctctgagcaccgctagaagctggagctacattgtggagaccagcaactccgacaatggcacatgctaccccggcgattttatcaactacgaggaactgagggagcagctcagctccgtctccagcttcgagagattcgaaatcttccccaagacatccagctggcccaaccatgactccaacaagggagtgacagctgcttgccctcacgctggcgccaaaagcttctacaagaacctcatttggctggtgaagaagggcaactcctatcccaagctgaatcaaacatatatcaacgacaagggcaaggaagtgctggtgctgtggggcattcaccaccctcccaccaccgctgatcagcagagcctctaccagaatgccgacgcctatgtgtttgtgggaacctctaggtactccaagaagttcaagcccgagatcgccacaaggcccaaggtgagagaccaagagggaagaatgaactactactggacactggtggagcccggcgataagattaccttcgaggccaccggcaatctggtcgtccccagatatgccttcaccatggagagaaacgccggcagcggaatcattatctccgacacccccgtgcacgattgcaacaccacatgccagaccgccgagggcgccatcaacacaagcctcccttttcagaacgtgcaccccgtgacaatcggcaagtgtcctaggtacgtcaagagcaccaagctcagactggccaccggactgaggaacgtgccctccatccagtctaggggactgtttggagccattgccggctttattgagggaggatggaccggcatggtcgatggctggtacggctaccaccatcagaatgaacaaggctccggatacgctgccgacctcaagagcacacagaatgccatcgacaagatcacaaataaagtcaacagcgtgatcgagaagatgaacacccagtttaccgccgtcggcaaagagttcaaccacctcgagaagaggatcgagaacctcaacaagaaggtggacgacggctttctggatatcgacacctatctggctgagctgctggtgctgctcgagaatcagtggacactgctgtatcatgattccaacgtcaagaatctgtacgagaaggtgagaaaccaactgaagaacaacgccaaggagatcggaaacggctgcttcgagttttaccacaagtgcgataatacatgcatggagagcgtcaagaacggcacctacgactatcccaagtactccgaagaggctaagctcaatagagaaaagatcgacggcgtcggcagcgagaatctctatttccaaggcggctccaagggctatattcccgaggctcctagagacggacaagcctatgtgaggaaggatggcgaatgggtgctcctctccacattcctcggacaccaccatcaccaccactgatga

SEQ ID NO: 16 - DNA, 1704 bp, synthetic construct
  source 1..1704: mol_type = other DNA
atgaaggctattctggtcgtcctcctctacaccttcaccaccgctaatgccgacacactgtgcatcggctaccatgccaacaacagcaccgatacagtggacaccgtgctggagaaaaacgtgaccgtcacacacagcgtgaatctgctggaggacaagcacaatggcaagctgtgcaagctgggcggagtggcccctctgcatctcggcaagtgtaacatcgctggatggattctgggcaatcccgagtgcgagtctctgagcacagctagaagctggagctacattgtggagaccagcaacagcgacaacggcacatgttaccccggagacttcatcaattacgaggagctgagagaacagctcagctccgtgtccagctttgagagattcgagatcttccccaagacctccagctggcccaaccacgacagcaataagggagtgaccgctgcttgtccccacgctggcgccaagagcttctacaaaaacctcatctggctggtgaagaagggcaactcctaccccaaactcaaccagacctatattaacgataaaggaaaggaagtgctggtgctctggggcatccaccaccctcccaccaccgctgaccaacagagcctctaccagaatgctgacgcctatgtctttgtgggaacctctaggtactccaagaagttcaagcccgagattgctacaaggcccaaggtgagagaccaagagggaagaatgaattactactggacactggtggagcccggcgacaaaatcaccttcgaagccaccggcaacctcgtggtccccagatacgccttcaccatggagaggaacgctggctccggaatcatcatttccgacacccccgtgcacgactgcaatacaacatgccagaccgccgagggcgccatcaatacctctctgcccttccagaatgtgcaccccgtgaccatcggcaagtgtcctaggtacgtcaagagcaccaagctgagactggccaccggactgaggaacgtgccctccatccagtctaggggactgttcggagccattgccggattcatcgagggcggctggaccggaatggtggacggatggtacggctaccaccaccaaaacgagcaaggctccggatacgccgctgatctgaagagcacacagaacgccatcgacaagatcaccaatatggtcaacagcgtgattgaaaagatgaacacccagttcaccgccgtgggaaaagagttcaaccatctcgagaagaggatcgagaacctcaacaagaaggtggacgatggcttcctcgacatcgatacctatctggctgagctgctggtgctgctgctcaatcaatggacactgctgtaccacgactccaacgtcaagaatctgtacgagaaggtgagaaatcagctcaaaaacaacgccaaagagatcggcaacggctgcttcgagttttatcacaagtgcgataacacatgcatggagtccgtgaagaacggcacctacgactatcccaagtactccgaagaggccaagctcaatagagagaagattgacggagtgggctccgagaacctctacttccaaggcggctccaaaggctacatccccgaggcccctagagatggacaagcctacgtgagaaaggacggagagtgggtgctcctcagcacatttctgggacatcatcatcaccaccactgatga

SEQ ID NO: 17 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNLLIGKTNEKFHQIEKEFSEVEGRVQDLEKYVEDTKIDLWSYNAELLVALMNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 18 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNMLIGKTNEKFHQIEKEFSEVEGRVQDLEKYVEDTKIDLWSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 19 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGILNFLIGKTNEKFHQIEKEFSEVEGRVQDLEKYVEDTKIDLWSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 20 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGVLNILIGKTNEKFHQIEKEFSEVEGRVQDLEKYVEDTKIDLWSYNAELLVALMNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 21 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEPEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 22 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYIAELLVALENQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 23 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGFFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQLTIALTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 24 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFMKTMKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 25 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNLLIGKTNEKFHQIEKEFSEVEGRVQDLEKYVEDTKIDLDSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 26 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNLLIGKTNEKFHQIEKEFSEVEGRVQDLEKYVEDTKIDLDSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 27 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNLLIGKTNEKFHQIEKEFSEVEGRVQDLEKYVEDTKIDLDSYNAELLVALMNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 28 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGVLNLLIGKTNEKFHQIEKEFSEVEGRVQDLEKYVEDTKIDLDSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 29 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGVLNLLIGKTNEKFHQIEKEFSEVEGRVQDLEKYVEDTKIDLDSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 30 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGMLNLLIGKTNEKFHQIEKEFSEVEGRVQDLEKYVEDTKIDLDSYNAELLVALVNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 31 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGMLNLLIGKTNEKFHQIEKEFSEVEGRVQDLEKYVEDTKIDLDSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 32 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGMLNLLIGKTNEKFHQIEKEFSEVEGRVQDLEKYVEDTKIDLDSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 33 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNMLIGKTNEKFHQIEKEFSEVEGRVQDLEKYVEDTKIDLDSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 34 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNFLIGKTNEKFHQIEKEFSEVEGRVQDLEKYVEDTKIDLDSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 35 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNFLIGKTNEKFHQIEKEFSEVEGRVQDLEKYVEDTKIDLDSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 36 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGILNFLIGKTNEKFHQIEKEFSEVEGRVQDLEKYVEDTKIDLDSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 37 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNILIGKTNEKFHQIEKEFSEVEGRVQDLEKYVEDTKIDLDSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 38 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGLLNILIGKTNEKFHQIEKEFSEVEGRVQDLEKYVEDTKIDLDSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 39 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGVLNILIGKTNEKFHQIEKEFSEVEGRVQDLEKYVEDTKIDLDSYNAELLVALLNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 40 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGVLNILIGKTNEKFHQIEKEFSEVEGRVQDLEKYVEDTKIDLDSYNAELLVALINQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 41 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGVLNILIGKTNEKFHQIEKEFSEVEGRVQDLEKYVEDTKIDLDSYNAELLVALMNQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 42 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEPEGRVQDLEKYVEDTKIDLDSYNAELLVALENQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 43 - protein, 16 aa, Influenza A
MKTIIALSNILCLVFA

SEQ ID NO: 44 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacctgctgatcggcaagaccaacgagaagttccatcagatcgagaaggagttcagcgaggtggagggcagagtgcaagacctggagaagtacgtggaggacaccaagatcgacctgtggagctacaacgccgagctgctggtggccctgatgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 45 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacatgctgatcggcaagaccaacgagaagttccatcagatcgagaaggagttcagcgaggtggagggcagagtgcaagacctggagaagtacgtggaggacaccaagatcgacctgtggagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 46 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcatcctgaacttcctgatcggcaagaccaacgagaagttccatcagatcgagaaggagttcagcgaggtggagggcagagtgcaagacctggagaagtacgtggaggacaccaagatcgacctgtggagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 47 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcgtgctgaacatcctgatcggcaagaccaacgagaagttccatcagatcgagaaggagttcagcgaggtggagggcagagtgcaagacctggagaagtacgtggaggacaccaagatcgacctgtggagctacaacgccgagctgctggtggccctgatgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 48 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgagcccgagggcagagtgcaagacctggagaagtacgtggaggacaccaagatcgacctgtggagctacaacgccgagctgctggtggccctggagaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 49 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagagtgcaagacctggagaagtacgtggaggacaccaagatcgacctgtggagctacatcgccgagctgctggtggccctggagaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 50 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcttcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagagtgcaagacctggagaagtacgtggaggacaccaagatcgacctgtggagctacaacgccgagctgctggtggccctggagaatcagctgaccatcgccctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 51 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgaggtggagggcagagtgcaagacctggagaagtacgtggaggacaccaagatcgacctgtggagctacaacgccgagctgctggtggccctggagaatcagcacaccatcgacctgaccgacagcgagatgaacaagctgttcatgaagaccatgaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 52 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 53 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 54 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 55 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcgtgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 56 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcgtgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 57 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcatgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctggtgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 58 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcatgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 59 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcatgctgaacctgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 60 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacatgctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 61 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacttcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 62 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacttcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 63 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcatcctgaacttcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 64 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacatcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 65 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcctgctgaacatcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 66 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcgtgctgaacatcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgctgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 67 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcgtgctgaacatcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatcaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 68 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcgtgctgaacatcctgatcggcaagaccaacgagaagttccatcagatcgagaaggagttcagcgaggtggagggcagagtgcaagacctggagaagtacgtggaggacaccaagatcgacctggacagctacaacgccgagctgctggtggccctgatgaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 69 - DNA, 1707 bp, synthetic construct
  source 1..1707: mol_type = other DNA
atgaagaccatcatcgccctgagcaacatcctgtgcctggtgtttgctcagaagatccccggcaacgacaacagcaccgccaccctgtgcctcggccaccacgccgtgcccaacggcactatcgtaaagacgattaccaacgacagaatcgaggtgaccaacgccaccgagctggtgcagaacagcagcatcggcgagatctgcgacagcccccatcagatcctggacggcggcaactgcaccctgatcgacgccctgctgggcgaccctcagtgcgacggctttcagaacaaggagtgggacctgttcgtggagagaagcagagccaacagcaactgctacccctacgatgtgccggactacgctagcctgagaagcctggtggctagcagcggcaccctggagttcaagaacgagagcttcaactggaccggcgtgaagcagaacggcacatctagcgcctgcatcagaggcagcagcagcagcttcttcagcagactgaactggctgacaagcctgaacaacatctaccccgctcagaacgtgaccatgcccaacaaggagcagttcgacaagctgtacatctggggcgtgcaccaccccgacaccgacaagaatcagatcagcctgttcgctcagagcagcggcagaatcaccgtgagcaccaagagatctcagcaagccgtgatccccaacatcggcagcagacctagaatcagagacatccctagcagaatcagcatctactggaccatcgtgaagcctggtgacatcctgctgatcaacagcaccggcaacctgatcgcccctagaggctacttcaagatcagaagcggcaagagcagcatcatgagaagcgacgcccccatcggcaagtgcaagagcgagtgcatcacccccaacggaagcatccccaacgacaagccctttcagaacgtgaacagaatcacctacggcgcctgccctagatacgtgaagcagagcaccctgaagctggccaccggcatgagaaacgtgcccgagaagcagacaagaggcatcttcggcgccatcgccggcttcatcgagaacggctgggagggcatggtggacggctggtacggcttcagacatcagaacagcgagggcagaggccaagccgccgacctcaagagcacccaagccgccatcgatcagatcaacggcaagctgaacagactgatcggcaagaccaacgagaagttccatcagatcgagaaggagttcagcgagcccgagggcagagtgcaagacctggagaagtacgtggaggacaccaagatcgacctggacagctacaacgccgagctgctggtggccctggagaatcagcacaccatcgacctgaccgacagcgagatgaacaagctgttcgagaagaccaagaagcagctgagagagaacgccgaggacatggggaacggctgcttcaagatctaccacaagtgcgacaacgcctgcatcggcagcatcagaaacgagacctacgaccacaacgtgtacagagacgaggccctgaacaacagatttcagatcaagggcgtgggcagcgagaacctgtacttccaaggcggcagcaagggctacatccccgaggcccctagagacggccaagcctacgtgagaaaggacggcgagtgggtgctgctgagcaccttcctggggcatcaccatcaccatcactgatga

SEQ ID NO: 70 - protein, 566 aa, synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVGSENLYFQGGSKGYIPEAPRDGQAYVRKDGEWVLLSTFLGHHHHHH

SEQ ID NO: 71 - protein, 567 aa, synthetic construct
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVGSENLYFQGGSKGYIPEAPRDGQAYVRKDGEWVLLSTFLGHHHHHH

SEQ ID NO: 72 - protein, 520 aa, Influenza A
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGV

SEQ ID NO: 73 - protein, 521 aa, Influenza A
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGV